(19) United States
(12) Patent Application Publication
(10) Pub. No.: US 2026/0124194 A1
(43) Pub. Date: May 7, 2026

(54) VELUSETRAG FOR USE IN THE TREATMENT OF CHRONIC INTESTINAL PSEUDO-OBSTRUCTION (CIPO)

(71) Applicant: ALFASIGMA S.P.A., Bologna (IT)

(72) Inventors: Loredana VESCI, Pomezia (RM) (IT); Maria GRIMALDI, Bologna (IT); Fabrizio GIORGI, Pomezia (RM) (IT); Roberto GIOVANNINI, Spilamberto (MO) (IT); Michelangelo BARONE, Bologna (IT)

(73) Assignee: ALFASIGMA S.P.A., Bologna (IT)

(21) Appl. No.: 19/105,498

(22) PCT Filed: Sep. 20, 2023
(86) PCT No.: PCT/EP2023/075925

§ 371 Date: Feb. 21, 2025

(30) Foreign Application Priority Data
Sep. 20, 2022 (EP) 22196573.4

(51) Int. Cl. A61K 31/4709; A61K 9/00; A61P 1/00; A61P 1/04; A61P 25/02

(52) U.S. Cl. CPC A61K 31/4709; A61K 9/0053; A61P 1/00; A61P 1/04; A61P 25/02

(57) ABSTRACT

The invention relates to 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide (velusetrag) or a pharmaceutically acceptable salt thereof for use in a method of treating idiopathic chronic intestinal pseudo-obstruction (CIPO), neuropathic chronic intestinal pseudo-obstruction, or chronic intestinal pseudo-obstruction being secondary to neurodegeneration or to demyelinating conditions in a patient suffering from said diseases or conditions.

### CROSS-REFERENCE TO RELATED APPLICATIONS

This application is a 371 of PCT/EP2023/075925, filed Sep. 20, 2023, which claims the benefit of European Patent Application No. 22196573.4, filed Sep. 20, 2022.

### FIELD OF THE INVENTION

The present invention relates to velusetrag (1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide), pharmaceutically acceptable salts thereof as well as compositions comprising said compound(s) for use in the treatment of chronic intestinal pseudo-obstruction (CIPO), particularly neuropathic CIPO or idiopathic CIPO. Hence, the present invention is in the field of methods of treating altered gastrointestinal motility conditions and disorders, such as chronic intestinal pseudo-obstruction (or colonic pseudo-obstruction) and disorders and conditions associated with constipation, for example constipation associated with the use of opiate painkillers, constipation post-surgery and constipation associated with neuropathic disorders and other conditions.

### BACKGROUND OF THE INVENTION

Pseudo-intestinal obstruction is a rare and severe condition characterized by disordered peristalsis with symptoms of intestinal obstruction, but without signs of mechanical obstruction. The disorder is caused by abnormalities of the enteric neuromusculature and/or its autonomic innervation; it represents the most severe form of gastrointestinal dysmotility with debilitating and life-threatening consequences.

When the condition persists for more than 6 months, chronic intestinal pseudo-obstruction (CIPO) occurs, one of the most important causes of chronic intestinal failure in both pediatric (15%) and adult (20%) cases. As affected individuals are often unable to sustain a normal oral diet and maintain a proper body weight, a severe clinical picture characterized by disabling digestive symptoms is established in the patient, which contributes to the severe deterioration of the patient's quality of life, leading to death.

A distinction is made between primary and secondary forms of CIPO. Primary CIPO is due to an intrinsic defect (congenital or acquired) and can be classified according to the type of damage (that could be associated): visceral myopathy (linked to muscle damage; e.g. MNGIE: mitochondrial neuro-gastrointestinal neuropathy, MMIHS: megacystis-microcolon-intestinal hypoperistalsis syndrome, idiopathic), visceral neuropathy (linked to damage to the autonomic nerves; e.g.: developmental anomaly of the myenteric plexus, Hirschsprung's disease, sequel of necrosing enterocolitis, idiopathic) or mesenchymopathy (linked to damage to Cajal cells in the digestive tract).

The etiological classification of CIPO has been evolving with the discovery of new genetic entities, in particular through the study of familial forms. Examples of involved genes are TYMP gene (mutated in MNGIE), ACTG2 gene (mutated in megacystis-microcolon-hypoperistalsis syndrome, MMIHS), SGOL1 gene (mutated in chronic atrial and intestinal arrhythmia syndrome, CAID), POLG gene (mutated in Alpers' disease). Despite that, the etiology of the majority of cases of CIPO is currently unknown and these cases are called idiopathic. They are thus included in the primary CIPO grouping.

Secondary CIPO is linked to an underlying systemic neurological, endocrine and connective tissue diseases or malignancies. In these diseases the intestinal motility could be affected due to the involvement of the autonomic nervous system (stroke, encephalitis, orthostatic hypotension), the nervous system of the intestinal wall (paraneoplastic syndromes, viral infections, iatrogenic, diabetes, Chagas' disease, Von Recklinghausen's disease), the muscular layer of the intestinal wall (myotonic dystrophy, progressive systemic sclerosis), the mixed enteric nervous system and the smooth muscle layer (scleroderma, dermatomyositis, amyloidosis, Ehlers-Danlos syndrome, jejunal diverticulosis, radiation enteritis) and finally unknown mechanisms (hypothyroidism, hypoparathyroidism, pheochromocytoma, antidepressants, anti-neoplastics, bronchodilators) [Antonucci A. et al. Chronic intestinal pseudo-obstruction.” World journal of gastroenterology 2008; 14:2953-61; Zhu C. Z. et al. Latest developments in chronic intestinal pseudo-obstruction. World J Clin Cases. 2020; 8:5852-5865; Billiauws L. et al. Small intestine motility disorders: Chronic intestinal pseudo-obstruction. J Visc Surg. 2022; 159(1S):S22-S27].

There are no clear epidemiological data; it has been estimated that in the USA about 100 infants per year are affected by intestinal pseudo-obstruction; the incidence among adults is 0.2 (male) and 0.24 (female) per 100.000 patients/year and its prevalence in adults has been estimated to be 0.2 to 0.9/100,000 population [Di Nardo G. et al. Pharmacological and nutritional therapy of children and adults with chronic intestinal pseudo-obstruction. Expert Review of Gastroenterology & Hepatology. Volume 17, 2023—Issue 4].

Generally, the most commonly reported symptoms are non-colic abdominal pain and distension (80%), persistent and aggravated by feeding. These symptoms are generally localized in the umbilical or upper abdominal regions and gradually spread to involve the entire abdomen. Other typical symptoms are nausea (75%), vomiting (40%-50%), constipation (40%) and diarrhoea (20%-30%).

Approximately 30% of patients in association with the typical symptoms also present with small intestinal bacterial overgrowth (SIBO); the condition contributes to further mucosal damage, steatorrhoea, diarrhoea and intestinal damage, with chronically dilated loops of the intestine contributing to malabsorption, vitamin deficiency and weight loss. Extraintestinal manifestations could be possible (e.g., bladder and ureteral in MMIHS; ophthalmoplegia, ptosis and peripheral polyneuropathy in MNGI; depression or other psychological disorders due to the long course of the disease).

The typical clinical manifestation of CIPO is the recurrence of pseudo-obstructive episodes, characterized by abdominal pain, abdominal distention and inability to defecate, with or without vomiting, resembling a mechanical sub-occlusion. An important diagnostic marker of this pathological condition during acute episodes is the radiologic evidence of the presence of distended bowel loops and air-fluid level in the upright position. In the most severe cases intestinal loops are chronically distended and air-fluid levels are detected.

Between acute episodes, patients could be asymptomatic or suffer from variable digestive symptoms, mainly related to the location and extent of the gastrointestinal tract involved.

Due to the CIPO symptomatic overlap with severe forms of other digestive disorders, and the lack of biomarkers, patients often undergo useless and potentially dangerous abdominal surgery before the cause of recurrent subacute obstructive episodes is suspected. The average time for a correct final diagnosis is 8 years. Diagnosis of CIPO is mainly clinic, supported by radiographic documentation of dilated bowel with air-fluid level, after exclusion of organic lesions occluding gut lumen, as detected by radiologic and/or endoscopic investigations. It's important to identify possible causes of secondary forms. Intestinal manometry could be useful to differentiate mechanical from functional obstruction and to recognize the underlying pathophysiological mechanism. Full thickness biopsy should be obtained from dilated or non dilated tracts of the alimentary canal in all patients with suspected CIPO who undergo surgery for unexplained occlusive episodes [Antonucci A. et al. Chronic intestinal pseudo-obstruction.” World journal of gastroenterology 2008; 14:2953-61].

The primary goal of CIPO therapy is to increase gastrointestinal motility, improve nutritional status and maintain a stable intestinal environment. In patients with secondary CIPO, of course, the primary disease must be actively treated to remove its cause.

In cases of acute intestinal pseudo-obstructive episodes, the use of surgery should be minimized because the operation can inhibit intestinal peristalsis and even induce further intestinal failure; the re-intervention rate in such cases is high.

For the treatment of visceral pain in CIPO patients, particularly chronic pain, low-dose tricyclic antidepressants and gabapentin are used, while opioids are not recommended due to their inhibition of gastrointestinal peristalsis. However, as the disease progresses, approximately 25% of patients gradually increase their tolerance to analgesic drugs, and the involvement of a team that also includes pain specialists and psychologists is recommended for appropriate therapy management [Stanghellini V. et al. Natural History of Chronic Idiopathic Intestinal Pseudo-Obstruction in Adults: A Single Centre Study, Clinical Gastroenterology and Hepatology, 2005; 3:449-458].

To counteract concomitant SIBO, the most common complication of chronic intestinal dilatation with bacterial concentrations generally above 103-105 CFU/mL, oral antibiotic therapy is prescribed and there are antibiotic-based treatment plans to choose from, such as amoxicillin-clavulanic acid (500 mg, tid), ciprofloxacin (500 mg, bid), doxycycline (100 mg, bid), metronidazole (250 mg, tid), neomycin (500 mg, bid), rifaximin (550 mg, bid) and tetracycline (250 mg, qid). Usually, antibiotics are prescribed for 7-10 days per month, with the type of antibiotic changed every month for 5-6 months to avoid resistance phenomenon.

Also, faecal bacterial transplantation has been proposed as a new approach for the treatment of CIPO. Studies have shown that faecal bacterial transplantation significantly relieves patients' abdominal distension and abdominal pain, increases their tolerance to enteral nutrition and prevents and treats related SIBO [Gu L. et al. Serial Frozen Fecal Microbiota Transplantation in the Treatment of Chronic Intestinal Pseudo-obstruction: A Preliminary Study. J Neurogastroenterol Motil. 2017; 23:289-297].

Intestinal transplantation may be life-saving in children, but is indicated only in patients in whom long-term parenteral nutrition cannot be performed or continued safely including patients who develop liver complications due to parenteral nutrition, have difficult central line access, or have poor quality of life and worsening pain despite aggressive medical management [Camilleri et al. Chronic Intestinal Pseudo-obstruction: Management, UpToDate, Feb. 17, 2022 2022].

As CIPO is characterized by an impairment of the propulsive activity in the intestinal tract, in the absence of any effective and resolutive medical treatment for the condition, drugs are commonly used to promote gastrointestinal motility, such as amoxicillin-clavulanic acid [Gomez R. et al. Effect of Amoxicillin/Clavulanate on Gastrointestinal Motility in Children, Journal of Pediatric Gastroenterology and Nutrition 2012; 54:780-784] and erythromycin, a macrolide antibiotic and motilin receptor activator [Emmanuel A. V. et al. Erythromycin for the treatment of chronic intestinal pseudo-obstruction: description of six cases with a positive response. Aliment Pharmacol Ther. 2004; 19:687-94]. However, a long-term treatment with erythromycin does not appear to be feasible due to tachyphylaxis (related to down regulation of motilin receptors) [Dhir R, Richter J E. Erythromycin in the short- and long-term control of dyspepsia symptoms in patients with gastroparesis. J Clin Gastroenterol 2004; 38:237-42].

There is still insufficient evidence for the treatment of CIPO with metoclopramide and domperidone, two antiemetic and prokinetic drugs, and the former carries the risk of tardive dyskinesia when used long-term. Acetylcholinesterase inhibitors (neostigmine, 8 mg/d, intravenous injection; pyridostigmine, 20 mg/d, oral administration) have also been used in adults with CIPO.

Serotonin (5-HT) is a neurochemical, which has been implicated in the control of gut motility; however, the functional role of endogenous 5-HT remains to be fully cleared, since depletion of neuronal or mucosal 5-HT has little or no effect on gut motility. On the other hand, the role of endogenous 5-HT produced by enterochromaffin cells in regulating gut motility remains obscure.

5-HT receptors are widely expressed within the gastrointestinal tract, and 5 of the 7 known families, 5-HT₁, 5-HT₂, 5-HT₃, 5-HT₄ and 5-HT₇ receptors, are expressed in the gut and can affect gut functions. The 5-HT₃ and 5-HT₄ receptor subtypes have been most extensively studied in the gut and have been targeted for the treatment of diarrhea and constipation, respectively.

Further, it has been described that endogenous 5-HT can act as a modulator of intestinal motility via activation of 5-HT₃ and 5-HT₄ receptors in the enteric nervous system (ENS). Within this tightly controlled system, disturbances in these regulatory mechanisms have been associated with gut motility disorders [Waclawiková B. et al. Gut bacteria-derived 5-hydroxyindole is a potent stimulant of intestinal motility via its action on L-type calcium channels. PLOS Biol. 2021; 19 (1): e3001070].

Prucalopride is a highly selective 5-HT₄ receptor agonist lacking cardiotoxicity, exerting significant neuroprotection in human enteric neurons [Bianco F. et al. Prucalopride exerts neuroprotection in human enteric neurons. Am J Physiol Gastrointest Liver Physiol. 2016; 310: G768-75], which has shown promising results in children and adults with acute and chronic intestinal pseudo-obstruction. The use of prucalopride in children with acute intermittent or chronic intestinal pseudo-obstruction is safe, effective and well tolerated [Mutalib M. et al. Prucalopride in intestinal pseudo-obstruction, paediatric experience and systematic review. Acta Gastroenterol Belg. 2021; 84:429-434].

Furthermore, Emmanuel A. V. et al. describe a phase II, double-blind, placebo-controlled, two-treatment four periods cross-over trial wherein the clinical safety, tolerability and the efficacy of prucalopride in improving the symptoms associated with CIPO was investigated. Subjects were treated for 4 periods of 12 weeks each with either prucalopride 2 mg (2 periods) or placebo (2 periods). There were no wash-out periods. In total, 7 subjects were randomized; 2 were assigned to the PLA-PRU-PLA-PRU, 2 to the PRU-PLA-PRU-PLA, 2 to the PLA-PRU-PRU-PLA, and 1 to the PRU-PLA-PLA-PRU sequence group. Three patients discontinued the study after the first period: one due to serious adverse events believed to be unrelated to the study medication (feeding line infection, sepsis, bronchopneumonia and malnutrition), and two due to withdrawal of consent (one each on placebo and prucalopride). The other 4 patients completed the study. Symptoms analyzed were pain, vomiting, nausea and bloating. The study suffers from the limited number of participants; however, results of this long-term study have shown that prucalopride alleviated CIPO symptoms: prucalopride significantly improved pain in three of four patients, nausea in two, vomiting in one, bloating in four. The number of analgesia intakes decreased substantially during treatment with prucalopride compared with the placebo periods. However, prucalopride did not affect stool frequency and consistency, with no evidence of a major prokinetic effect on gastrointestinal transit. The incidence of pseudo-obstructive episodes in this study with CIPO patients were not analyzed. [Emmanuel A. V., et al. Randomised clinical trial: the efficacy of prucalopride in patients with chronic intestinal pseudo-obstruction—a double-blind, placebo-controlled, cross-over, multiple n=1 study. Aliment Pharmacol Ther. 2012; 35:48-55; NCT00793247].

Other 5-HT₄ receptor agonists, such as cisapride and tegaserod, are effective but they have been banned because of related fatal arrhythmias.

There are several variants of the 5-HT₄ receptor due to alternative splicing of the corresponding genes which explains the different effects of the used reference compounds with respect to agonistic/antagonistic activity on 5-HT4 splice variants. This accounts for a high level of unpredictability of the targeting of the various variants of said receptor in terms of therapeutic effects. The importance of variations introduced by splicing for receptor pharmacology may help in the understanding of conflicting results seen with 5-HT4 receptor ligands in different model systems [Takaki M. et al. The 5-hydroxytryptamine 4 Receptor Agonist-induced Actions and Enteric Neurogenesis in the Gut. J Neurogastroenterol Motil. 2014; 20:17-30].

It is evident that so far still an unmet medical need remains for patients with CIPO. Despite extensive research, pharmacological treatment options remain limited and are often associated with serious side effects. This means that so far there have been no satisfactory treatments of CIPO with a view to effectively improving its main symptoms. As outlined above, the prior art attempts for the treatment of CIPO only gave unsatisfactory results or were associated with severe side effects. In particular, there has been no treatment showing a prokinetic effect. Moreover, there has been no treatment targeting the neurodegeneration underlying the impairment of the neuro-enteric system and gut dysmotility condition.

Velusetrag is a highly selective 5-hydroxytryptamine subtype 4 (5-HT₄) receptor agonist with prokinetic activity. The chemical name of velusetrag is 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide and its chemical structure is shown below in Formula I:

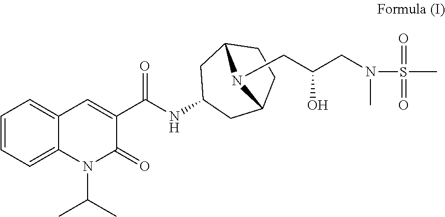 [SMILES: CC(C)n1c(=O)c(C(=O)N[C@H]2C[C@H]3CC[C@@H](C2)N3C[C@@H](O)CN(C)S(C)(=O)=O)cc2ccccc21]

Velusetrag was previously disclosed in U.S. patent application Ser. No. 11/100,113, filed on Apr. 6, 2005; corresponding to EP 1 735 304, and the relative crystalline form disclosed in U.S. patent application Ser. No. 11/398,119, filed Apr. 5, 2006, corresponding to EP 1874766.

In both pre-clinical models and healthy humans, a robust gastrointestinal prokinetic effect in vivo of velusetrag has been confirmed [Smith J. A. M. et al. The in vitro pharmacological profile of TD-5108, a selective. Naunyn-Schmiedeberg's Arch Pharmacol. 2008; 378:125-137; Beattie D. T., et al. The in vivo gastrointestinal activity of TD-5108, a selective 5-HT₄ receptor agonist with high intrinsic activity. Naunyn-Schmiedeberg's Arch Pharmacol. 2008; 378:139-147].

Activation of 5-HT₄ receptors is associated with contraction of guinea pig isolated colonic longitudinal muscle and inhibition of electrically evoked or spontaneous contractions of human isolated colonic circular muscle. 5-HT₄ receptor agonism in vivo results in increased colonic transit in guinea pigs, oesophageal relaxation in rats, and enhancement of motility in the upper and lower GI tract of dogs. Velusetrag has been evaluated for the treatment of gastrointestinal (GI) motility disorders including chronic idiopathic constipation (CIC) and gastroparesis (GP).

WO 2015/175997 describes a method for treating or ameliorating the effects of a condition, i.e., autism, having altered serotonin transporter (SERT) activity that impairs the enteric nervous system and consequently intestinal motility. The method comprises administering a 5-HT4 agonist to the subject. Among the diseases with altered SERT activity, CIPO is also mentioned, and among the 5-HT4 agonists, velusetrag is disclosed but it does not specifically describe examples where velusetrag is used in CIPO.

Clinical study NCT00391820 has shown that patients having less than 3 spontaneous bowel movements (SBM)/week receiving 15, 30 and 50 mg daily for 4 weeks achieved statistically and clinically significant increases in weekly SBM frequency relative to those receiving placebo [Goldberg M. et al. Clinical trial: the efficacy and tolerability of velusetrag, a selective 5-HT4 agonist with high intrinsic activity, in chronic idiopathic constipation—a 4-week, randomized, double-blind, placebo-controlled, dose-response study. & Alimentary Pharmacology Therapeutics 2010; 31:1102-1112].

Ahn A. et al. discloses the prokinetic effect of velusetrag in the upper gastrointestinal tract by assessing gastric emptying (GE) time in both diabetic and idiopathic gastroparesis subjects randomized into 4 groups receiving respectively velusetrag 5 mg, 15 mg, 30 mg and placebo. The study results have shown that the used doses of velusetrag were effective to accelerate gastric emptying. All doses were well tolerated but significance levels were reached for the 30 mg dose [Ahn A. et al. Su 1426 Velusetrag improves gastric emptying time in subjects with diabetic or idiopathic gastroparesis. Gastroenterology 2015; 148: S-507].

Clinical study NCT02267525 and patent application WO 2019/027881 (corresponding to EP 18 756 041.2) teach the use of velusetrag or a pharmaceutically acceptable salt thereof in a method for preventing, alleviating, ameliorating, giving relief to, treating the core symptoms of gastroparesis consisting of postprandial fullness, early satiety, bloating, upper abdominal pain, epigastric burning, nausea and vomiting in diabetic or idiopathic human patients. The method comprises administering velusetrag in the specific dosage of 5 mg/day for a treatment period of one, two, four, eight or twelve weeks [Abell T. et al. Velusetrag improves gastroparesis both in symptoms and gastric emptying in patients with diabetic or idiopathic gastroparesis in a 12-week global phase 2B study. Abstract for oral presentation at DDW (Digestive Disease Week) Meeting, San Diego (CA). May 18, 2019. Abell T. et al. Efficacy of velusetrag treatment in patients with idiopathic gastroparesis: subgroup analysis of a phase 2b study, Abstract at UEG Week 2019, 26 Apr. 2019].

As other agonists of 5-TH4 receptor, velusetrag works by triggering the release of neurotransmitters such as acetylcholine from enteric motor neurons and calcitonin gene-related peptide from sensory neurons in the GI tract. By selectively activating 5-HT₄ receptors in the gastrointestinal tract, velusetrag enhances the peristaltic reflex, stimulates intestinal secretion, and inhibits visceral sensitivity.

The inventors have demonstrated that 5-HT4 activation induced by velusetrag enhanced a specific protective response in the different neuronal cells analyzed, including human enteric neurons. These findings open new avenues in the treatment of gastrointestinal disorders and homeostasis, especially in relation to neurodegeneration often underlying severe gut dysmotility.

### SUMMARY OF THE INVENTION

The present invention relates to velusetrag or a pharmaceutically acceptable salt thereof for use in a method of treating idiopathic chronic intestinal pseudoobstruction (CIPO), neuropathic chronic intestinal pseudo-obstruction or chronic intestinal pseudo-obstruction being secondary to neurodegeneration or being secondary to autoimmune conditions or secondary to connective tissue disorders or being secondary to demyelinating conditions.

According to one embodiment, the pharmaceutically acceptable salt is a hydrochloride salt. The invention moreover relates to the above use, wherein velusetrag is in crystalline form and/or hydrated form.

The invention, moreover, relates to the above use, wherein CIPO is the idiopathic CIPO, the neuropathic CIPO or the CIPO being secondary to neurodegeneration or being secondary to autoimmune conditions or secondary to connective tissue disorders or being secondary to demyelinating conditions, or any CIPO caused by diseases of the autonomic nervous system such as stroke, encephalitis, calcification of basal ganglia, orthostatic hypotension, one caused by diseases of intestinal wall nervous system such as paraneoplastic syndrome, viral infections, iatrogenic disorders, Hirschsprung's disease, Chagas' disease, Von Recklinghausen's disease, one caused by diseases of the intestinal wall muscle layer such as myotonic dystrophy, progressive systemic sclerosis or one caused by diseases of the mixed enteric nervous system and smooth muscle layer such as scleroderma, dermatomyositis, amyloidosis, Ehler-Danlos syndrome or one caused by an unknown mechanism such as hypothyroidism, hypoparathyroidism, pheochromocytoma, antidepressants drugs, antineoplastics or bronchodilatators or one caused by immune-mediated and connective tissue disorder or disease such as paraneoplastic disease (CNS neoplasms, lung microstoma, bronchial carcinoid, leyomyosarcomas, systemic lupus erythematosus).

The invention moreover relates to the above use, wherein the patient is a subject with CIPO. Preferably the patient is an adult or a pediatric subject.

The invention moreover related to the above use, wherein the patient is one having a history of chronic CIPO or CIPO secondary to neurodegenerative disease or demyelinating disease.

Moreover, the invention relates to the above use, wherein at least one of the symptoms of CIPO (e.g. vomiting, bloating, abdominal pain) or disease exacerbations (e.g. pseudo-obstructive episodes) is alleviated or ameliorated. Moreover, the invention relates to the above use, wherein at least one of the symptoms of CIPO selected from abnormal gastrointestinal motility, increased dilatation of the proximal colon and/or of the distal small intestine, modified intestinal contractility, ulceration, inflammation of the proximal colon and/or of the distal small intestine, pseudo-obstructive episodes and lethality is alleviated or ameliorated and/or wherein the number and/or frequency of CIPO-related and/or CIPO-caused hospitalizations is reduced.

Further to this, the invention relates to the above use, wherein velusetrag is administered in a dose amount ranging from 0.5 mg to 30 mg, preferably from 5 mg to 15 mg, based on the weight of the free base. These dosages are meant to be daily doses, for an average adult human patient. However, dosages can be varied and/or adapted in function of the degree of severity of the disease, the specific patient conditions, the specific administration route chosen.

The invention moreover relates to the above use, wherein treatment duration can be varied and/or adapted in function of the degree of severity of the disease or the specific patient conditions. Preferably the treatment is administered for at least 1 to 24 weeks, preferably at least 24 weeks, preferably for at least 2 weeks or at least 4 weeks, preferably at least 6 weeks, preferably at least 12 weeks, or at least 14 weeks, preferably at least 16 weeks, preferably at least 18 weeks, 20 weeks, 22 weeks. Preferably 24 weeks or at least 24 weeks, preferably 26 weeks, 28 weeks, 30 weeks or longer. The treatment may also be administered at repeated cycle over at least at least 1 to 24 weeks, preferably at least 24 weeks, preferably for at least 2 weeks or at least 4 weeks, preferably at least 6 weeks, preferably at least 12 weeks, or at least 14 weeks, preferably at least 16 weeks, preferably at least 18 weeks, 20 weeks, 22 weeks. Preferably 24 weeks or at least 24 weeks, preferably 26 weeks, 28 weeks, 30 weeks or longer.

Velusedrag is preferably administered in a dose of 15 mg daily, wherein the dose is taken once daily, preferably in the form of 3×5 mg tablets. The preferred administration route is oral administration.

In another embodiment of the invention, pharmaceutical compositions with different routes of administration of velusetrag to the human patient are encompassed. The routes of administration comprise, inter alia, oral, parenteral, buccal, sublingual, rectal, intraperitoneal, or endotracheal routes of administration. For example, parenteral administration may be by infusion, injection, or implantation. Parenteral may also include percutaneous administration via subcutaneous, intramuscular, intravenous, transdermal, or by implantation routes. If velusetrag is administered parenterally, it may be in the form of a liquid, solid or gel. Similarly, if velusetrag is administered orally, it may be in the form of a liquid, capsule, tablet, chewable tablet or dissolvable film.

In one embodiment, the product comprises velusetrag in an amount from about 0.5 mg to about 30 mg labeled for treatment of symptoms of CIPO. In yet another embodiment, the product comprises velusetrag in an amount from about 0.5 mg to about 15 mg, from about 0.5 mg to about 5 mg, from about 5 mg to about 15 mg, or about 5 mg, or about 15 mg labeled for treatment of symptoms of CIPO.

In one embodiment, the invention provides a kit comprising using and dosing instructions on a package insert of a pharmaceutical product comprising velusetrag according to the invention. In another embodiment, the package insert instructs the patient to administer velusetrag for a period of treatment as indicated above.

Velusedrag may be administered in combination with other drugs not contraindicated with the administration of velusedrag. Preferably velusedrag is not administered together with opioids and/or other 5-HT4 receptor agonists (e.g., prucalopride, cisapride, clebopride, cinitapride).

### BRIEF DESCRIPTION OF DRAWINGS

In the following figures Normal=wild-type mice, G2, G3 and G4 groups are PrP-SCA7-92Q transgenic mice.

FIG. 1: shows dilatation measured as diameter (mm) of distal small intestine (DSI) and proximal colon of two murine models of CIPO treated with velusetrag 1 mg/kg and 3 mg/kg. 1A: Rb1cKO mice (CKO) ***P<0.001 vs. normal group. 1B: PrP-SCA7-92Q transgenic mice. *P<0.05, ***P<0.001; One way ANOVA; vs G2 (vehicle).

FIG. 2: shows histological analysis score (H&E total score as defined in Example 3) for DSI and proximal colon of two murine models of CIPO treated with velusetrag 1 mg/kg and 3 mg/kg. 2A: histology analysis score for DSI and proximal colon in velusetrag treated Rb1cKO mice model of CIPO *P<0.05, ***P<0.001; One way ANOVA; vs vehicle. 2B: histology analysis score for DSI and proximal colon in velusetrag treated PrP-SCA7-92Q transgenic mice model of CIPO, *P<0.05, ***P<0.001; T-test; vs G2.

FIG. 3: Protein levels of p-mTOR, mTOR, p-Akt, Akt and p-P70S6 in distal small intestine. Expression levels of p-mTOR and mTOR were normalized to Actin. Expression levels of p-Akt and Akt were normalized to Actin. Expression levels of p-P70S6 were normalized to Actin. The results are presented as the mean±SEM. *P<0.05, **P<0.01, ***P<0.001, vs vehicle group, n=10.

3A: refers to expression level fold change of the various proteins in the DSI of wild-type (normal) and Rb1cKO (CKO) mice treated with velusetrag 1 mg/kg and 3 mg/kg

3B: refers to expression level fold change of the various proteins in the DSI of wild-type (normal) and PrP-SCA7-92Q transgenic mice (G2, G3, G4) mice treated with velusetrag 1 mg/kg and 3 mg/kg.

FIG. 4: shows quantitative analysis of MAP2 neurons in DSI and proximal colon of two murine models of CIPO treated with velusetrag 1 mg/kg and 3 mg/kg. 4A: Rb1cKO mice. 4B: PrP-SCA7-92Q transgenic mice. The results are presented as the mean±SEM. *P<0.05, **P<0.01, ***P<0.001, vs vehicle group, n=10.

FIG. 5: Immunofluorescent for SOX10 and Hu+ neurons of DSI and colon in wild-type (normal) and Rb1cKO mice treated with Velusetrag (1 mg/kg or 3 mg/kg). Quantitative analysis of ganglia neurons in distal small intestine (A) and colon (B). Ratio of Glia/Hu+ neurons in distal small intestine (C) and colon (D). The results are presented as the mean±SEM. *P<0.05, **P<0.01, ***P<0.001, vs vehicle group, n=10.

FIG. 6: shows 5HT4 receptor mRNA expression in DSI and colon of velusetrag treated Rb1cKO mice (1 mg/kg or 3 mg/kg). The results are presented as the mean±SEM. *P<0.05, **P<0.01, ***P<0.001, vs vehicle group, n=10.

FIG. 7: Immunofluorescent colocalization of ataxin-7 intranuclear inclusions with Hu+ cytoplasmic markers in distal small intestine and colon. Quantitative analysis of Hu+ neurons counts in distal small intestine (A) and colon (B) of velusetrag treated PrP-SCA7-92Q transgenic mice (1 mg/kg and 3 mg/kg). Percentage of colocalized cell in Hu+ cell in distal small intestine (C) and colon (D). The results are presented as the mean±SEM. *P<0.05, **P<0.01, ***P<0.001, vs vehicle group, n=10.

FIG. 8: Immunofluorescent of ataxin-7 and nNOS+ cytoplasmic markers in distal small intestine and colon of velusetrag treated PrP-SCA7-92Q transgenic mice (1 mg/kg and 3 mg/kg). Quantitative analysis of nNOS+ neurons counts in distal small intestine (A) and colon (B). The results are presented as the mean±SEM. *P<0.05, **P<0.01, ***P<0.001, vs vehicle group, n=10.

FIG. 9: shows the quantitative analysis of CHAT+ neurons counts in distal small intestine (A) and colon (B) of velusetrag treated Rb1cKO mice (1 mg/kg and 3 mg/kg).

FIG. 10: Immunofluorescent colocalization of ataxin-7 intranuclear inclusions (in red) with CHAT+ cytoplasmic markers in distal small intestine and colon of velusetrag treated PrP-SCA7-92Q transgenic mice (1 mg/kg and 3 mg/kg). Quantitative analysis of CHAT+ neurons counts in distal small intestine (A) and colon (B) of velusetrag treated PrP-SCA7-92Q transgenic mice (1 mg/kg and 3 mg/kg). Percentage of colocalized cell in CHAT+ cell in distal small intestine (C) and colon (D). The results are presented as the mean±SEM. *P<0.05, **P<0.01, ***P<0.001, vs vehicle group, n=10.

FIG. 11: Immunofluorescence staining for Calretinin neurons of velusetrag treated PrP-SCA7-92Q transgenic mice (1 mg/kg and 3 mg/kg). Calretinin neurons of distal small intestine (A) and Calretinin neurons of colon (B). The results are presented as the mean±SEM. *P<0.05, **P<0.01, ***P<0.001, vs vehicle group, n=10.

FIG. 12: shows n-NOS and CHAT cytoplasm expression in proximal colon of velusetrag treated PrP-SCA7-92Q transgenic mice (1 mg/kg and 3 mg/kg). 12A: cytoplasm nNOS/Actin. 12B: cytoplasm CHAT/Actin.

FIG. 13: Body weight of PrP-SCA7-92Q transgenic mice 5 weeks after treatment.

FIG. 14: Immunofluorescent for SOX10 and Hu+ neurons of small intestine and colon in wild-type (normal) and PrP-SCA7-92Q transgenic mice treated with Velusetrag. Quantitative analysis of ganglia neurons in distal small intestine (A) and colon (B). Ratio of Glia/Hu+ neurons in distal small intestine (C) and colon (D). The results are presented as the mean±SEM. *P<0.05, **P<0.01, ***P<0.001, vs vehicle group, n=10.

FIG. 15: 5HT4 Receptor mRNA expression in distal small intestine and colon in wild-type (normal) and PrP-SCA7-92Q transgenic mice treated with Velusetrag. The results are presented as the mean±SEM. *P<0.05, **P<0.01, ***P<0.001, vs vehicle group, n=10.

FIG. 16: Clinical Study phase II scheme. Patients were randomized in 4 different arms having 4 different sequences of treatment. The study consisted of a screening period of up to 7 days (Day −7 to Day −1) followed by 4 periods of treatment of 4 weeks each wherein subjects were treated with either velusetrag (VEL) 15 mg (2 periods) or placebo (PLA) (2 periods) with a wash-out period of 2 weeks between treatment periods and 2 weeks of follow-up (total of approximatively 175 days). Scheduled visits (V=visit) at the corresponding days from the start of the study are also shown.

FIG. 17: shows the line plot of the mean of WGGSAIS of subjects treated with velusetrag or placebo by treatment (30 observed pairs) in mFAS1 population.

D7=Day 7; D14=Day 14; D21=Day 21; EoT=End of treatment; PLA=Placebo; PRE=Pre-treatment; VEL=Velusetrag; WO-D7=Wash-Out Day 7; WO-D14=Wash-Out Day 14.

Paired t-test: p-value=0.0310, 95% CI for the mean difference in changes between velusetrag and placebo, equal to −0.741; −0.038.

FIG. 18: shows the line plot of the mean of each individual symptom score of subjects treated with velusetrag or placebo by treatment in mFAS1 population.

D7=Day 7; D14=Day 14; D21=Day 21; EoT=End of treatment; mFAS1=modified Full Analysis Set 1; PLA=Placebo; PRE=Pre-treatment; VEL=Velusetrag; WO-D7=Wash-Out Day 7; WO-D14=Wash-Out Day 14. Only data that constitute pair evaluable for each individual symptom were considered in the analysis with the inclusion of wash-out Day 7 and wash-out Day 14. Paired t-test: p-value=0.0164

FIG. 19: shows the line plot of the mean of bowel habits score of subjects treated with velusetrag or placebo by treatment in mFAS1 population.

D7=Day 7; D14=Day 14; D21=Day 21; EoT=End of treatment; PLA=Placebo; PRE=Pre-treatment; VEL=Velusetrag; WO-D7=Wash-out Day 7; WO-D14=Wash-out Day 14. Only data that constitute pair evaluable for weekly bowel movements or complete evacuations are considered in the analysis with the inclusion of wash-out Day 7 and wash-out Day 14.

### DETAILED DESCRIPTION OF THE INVENTION

As outlined above, velusetrag is a compound of Formula I and forms a crystalline hydrochloride salt as shown in Formula II.

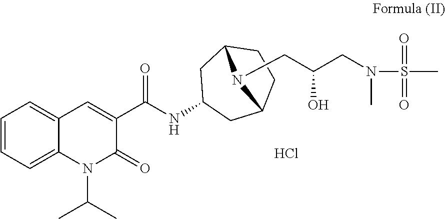 [SMILES: CC(C)n1c(=O)c(C(=O)N[C@H]2C[C@H]3CC[C@@H](C2)N3C[C@@H](O)CN(C)S(C)(=O)=O)cc2ccccc21 ; Cl]

The present invention basically relates to velusetrag for use in a method of treating idiopathic chronic intestinal pseudoobstruction (CIPO), neuropathic chronic intestinal pseudo-obstruction or chronic intestinal pseudo-obstruction being secondary to neurodegeneration or being secondary to autoimmune conditions or being secondary to connective tissue disorders or being secondary to demyelinating conditions. Moreover, the invention relates to the above use, wherein at least one of the symptoms of CIPO (e.g., vomiting, bloating, abdominal pain, constipation etc.) and or disease exacerbations (e.g., pseudo-obstructive episodes) is alleviated or ameliorated.

By said use the main symptoms of CIPO being abnormal gastrointestinal motility, increased dilatation of the proximal colon and/or of the distal small intestine, modified intestinal contractility, ulceration, inflammation of the proximal colon and/or of the distal small intestine and lethality can be alleviated or ameliorated. Additionally, the use of velusetrag according to the invention allows to reduce the need for “artificial food” (i.e., home-based or hospital-based parenteral nutrition). Velusetrag also reduces the number/frequency and duration of CIPO-related hospitalizations (i.e. hospitalizations that are a direct consequence of CIPO, directly result from CIPO and/or are complicated by CIPO), of CIPO-caused hospitalization (i.e. hospitalizations that are directly resulting from CIPO) and improves the quality of life.

To evaluate the preclinical pharmacodynamics of velusetrag in neuropathic CIPO, two types of models were used. One of these consists in Rb1 retinoblastoma knock out (Rb1cKO or CKO) mice, mutated for retinoblastoma in enteric nervous system. This model established by Fu et al. has allowed to highlight an intestinal pseudo-obstruction associated to an important neuronal disfunction and modified intestinal contractility [Fu M, et al. Retinoblastoma protein prevents enteric nervous system defects and intestinal pseudo-obstruction. J Clin Invest. 2013; 123:5152-5164]. Retinoblastoma Rb1 cKO mice died of intestinal pseudo-obstruction prematurely, so that 50% of mice was not alive by post-natal day P30. Distal Small Intestine (DSI) and proximal colon became dilated, and colon presented hard dark stool scybala starting from post-natal days P8 to P30.

As in mice, CIPO in patients is diagnosed when bowel motility defects cause functional, but not mechanical, obstruction, leading to abdominal distension, pain, malnutrition, and, in severe cases, dependence on parenteral nutrition or intestinal transplantation for survival. It is likely that diverse genetic, infectious, autoimmune, metabolic, and toxic insults all contribute to CIPO etiology.

It is known that intestinal motility is controlled by an interconnected intrinsic network of neurons and glia named enteric nervous system (ENS). Generally, the ENS forms from neural crest-derived cells that migrate through fetal bowel, proliferate extensively, and then exit the cell cycle differentiating into many different neuronal subtypes. In case of defects in some enteric neurons it is possible to put in danger life, even because neurons in the ENS must be present in the proper ratios to allow that the bowel works well. Signals controlling ENS precursor proliferation and cell cycle exit are not completely clear.

Starting from the above observations, mice cKO for Rb1 retinoblastoma were prepared because Rb1 is involved in the cell cycle exit by preventing cells from entering S phase, in the cell cycle checkpoints in S and G2/M phases, so to keep the development, terminal differentiation and tissue homeostasis and its mutation causes tissue specific defects. Actually, Rb1 inactivation in the ENS leads to a progressive, fatal defect in a subset of NO-producing myenteric neurons that inhibits bowel contraction. These cells undergo endoreplication and developed giant, irregularly shaped nuclei similar to those seen in progeria. In addition, Rb1 was also deleted in enteric glia and other types of enteric neurons that do not undergo endoreplication, highlighting differences in Rb1 dependence of distinct cell types within the ENS lineage.

In terms of translational evidences on this relevant Rb1 cKO mice model of CIPO, velusetrag after only two weeks of treatment, in addition to its capability to reduce the dilatation of proximal colon, was able to protect mice from lethality and to counteract glia activation as index of gut inflammation evident in cKO mice. The effect of velusetrag on reverting intestinal dilatation associated with a specific anti-inflammatory protective action could plausibly suggest a disease-modifying effect in addition to the prokinetic effect.

The other preclinical pharmacodynamics model consists in transgenic mice PrP-SCA7-92Q, also referred to in this description as human ATXN7 transgenic mice, that develop signs of intestinal pseudo-obstruction and visceral neuropathies [Clarke C. M. et al. Visceral neuropathy and intestinal pseudo-obstruction in a murine model of a nuclear inclusion disease. Gastroenterology. 2007; 133 (6): 1971-1978]. These animals show many aspects of the human polyglutamine neurodegenerative disorder, spinocerebellar ataxia type 7 (SCA7) and a subset of cholinergic enteric ganglion cells with nuclear inclusions comprised of transgene-derived ataxin-7 and a pathogenic polyglutamine expansion. Transgenic mice present a marked distension of the distal small intestine from a 13-week of age.

Ataxin-7 inclusions are evident in the nuclei of a subset of enteric ganglion cells and of myenteric neurons that co-express the cytoplasmic marker choline acetyltransferase. The density of calretinin-immunoreactive myenteric ganglion cells was also found significantly less in transgenic mice at 13 weeks in proximal colon. A loss of nerve fibers in the myenteric nerve plexus, and a delayed gastrointestinal transit were also observed. In this transgenic model, velusetrag delivered for 5 weeks by oral route once daily revealed to inhibit dilatation of distal small intestine to counteract the neurodegeneration of ChAT, nNOS and calretinin neurons and the appearance of ataxin-7 inclusions in enteric neurons.

Moreover, velusetrag increased the cytoplasmatic ChAT and nNOS levels in proximal colon thus suggesting a capability to modify the neuronal plasticity toward the normality and the gut motility.

The doses of velusetrag for both the preclinical murine models chosen for the treatment were calculated according to those administered during the clinical development in patients, i.e., 15 mg and 5 mg/patient [FDA Guidelines US FDA, 2005; Nair and Jacob, 2016].

Furthermore, in such two murine models the effect of velusetrag administration on the activation of the PI3K/AKT/mTOR signaling pathway was evaluated.

mTOR is a component of the protein complexes mTOR Complex 1 (mTORC1) and mTOR Complex 2 (mTORC2) that are ubiquitous throughout the body and control multiple functions such as gene transcription, metabolism, cell survival, and cell senescence. mTOR through its relationship with phosphoinositide 3-kinase (PI 3-K) and protein kinase B (Akt) and multiple downstream signaling pathways such as p70 ribosomal S6 kinase (p70S6K) and proline rich Akt substrate 40 kDa (PRAS40), promotes neuronal cell regeneration through stem cell renewal and oversees critical pathways such as apoptosis, autophagy, and necroptosis to foster protection against neurodegenerative disorders. Overall, mTOR is an essential neuroprotective pathway but must be carefully targeted to maximize clinical efficacy and eliminate any clinical toxic side effects. [Maiese K. Driving neural regeneration through the mammalian target of rapamycin. Neural Regen Res. 2014; 1; 9:1413-7.]

Diseases of the nervous system are often associated, if not due, to an alteration or disruption of homeostatic processes controlled by the glia. In the ENS, these roles are played by a unique population of peripheral neuroglia called enteric glia. Enteric glia regulates gastrointestinal motility through bidirectional communication with enteric neurons and contribute to the establishment of neuroinflammation. Glial mechanisms may contribute to gastrointestinal motility disturbances as supported by data from animal models showing that altered glial function disrupts motility promotes neurodegeneration during acute colitis and influences the immune response. However, the specific mechanisms by which enteric glia might contribute to motility disturbances remain largely uncharacterized. [Ahmadzai M. M. et al., J Clin Invest. 2022 Feb. 15; 132 (4): e149464]. The effect of velusetrag on neuronal and glial cells in the mouse models of in Rb1 retinoblastoma knock out (cKO) mice, showing intestinal pseudo-obstruction associated to neuronal disfunction and modified intestinal contractility and in the model of transgenic mice PrP-SCA7-92Q developing signs of intestinal pseudo-obstruction and visceral neuropathies, was assessed using immunohistochemical analysis on distal small intestine and proximal colon samples with antibody directed to the glial marker SOX10, and the neuronal protein HuCD.

The experiments results have shown that velusetrag administered for 14 days is able to reduce the expression of the relevant receptor, in particular in the Distal Small Intestine (DSI) region of the models for CIPO associated with neuronal disfunction and modified intestinal contractility.

RB1 cKO significantly induced dilatation in DSI and colon in mice. Very surprisingly, it has been found that in RB1 cKO mice treated with velusetrag at 1 mg/kg and 3 mg/kg, there was a trend inhibiting dilatation with statistic difference in colon after administration of 3 mg/kg. Increased dilatation in DSI and colon was observed also in Prp-SCA7-92Q transgenic mice compared with normal mice. After treatment with velusetrag 1 mg/kg or 3 mg/kg, the dilatation of DSI was improved significantly. Treatment with velusetrag 1 mg/kg or 3 mg/kg can reduced the dilatation in proximal colon.

The number of glia and glia/neurons ratio increased in DSI and colon of RB1 cKO vehicle group mice. Surprisingly, after treatment with velusetrag 3 mg/kg, the number of glia and glia/neurons ratio decreased significantly in DSI and proximal colon. In Prp-SCA7-92Q or human ATXN7, transgenic mice the ratio of glia to neuron in DSI and colon of vehicle group increased significantly. After treatment with velusetrag 1 mg/kg or 3 mg/kg the ratio of glia to neuron decreased significantly. The number of MAP2 stained neurons decreased in DSI and colon of RB1 cKO vehicle group. After treatment of velusetrag 1 mg/kg and 3 mg/kg, the number of MAP2 stained neurons increased in DSI and proximal colon. In Prp-SCA7-92Q transgenic mice the number of MAP2 stained neurons decreased in DSI and proximal colon of vehicle group. After treatment of velusetrag 1 mg/kg and 3 mg/kg, the number of MAP2 stained neurons increased in DSI and proximal colon.

The number of CHAT stained neurons decreased in DSI and colon of RB1 cKO vehicle group. After treatment of velusetrag 1 mg/kg and 3 mg/kg, the number of CHAT stained neurons increased in DSI and proximal colon.

In hematoxylin-eosin staining (HE), the DSI and colon showed distinct inflammation infiltration and ulcer both in RB1 cKO vehicle group and PrP-SCA7-92Q transgenic vehicle group. Surprisingly, the disease score of velusetrag 1 mg/kg and 3 mg/kg groups decreased significantly after treatment in both animal models of CIPO. 5HT4 receptor mRNA level of DSI and colon in RB1 cKO vehicle group increased but with no significant difference. After treatment with velusetrag 3 mg/kg, 5HT4 receptor mRNA level of DSI decreased significantly.

P-mTOR, p-AKT, p-P70S6, mTOR and AKT proteins increase in RB1 cKO vehicle group in which it showed significant increasing in p-Akt, p-P70S6 protein and p-AKT/AKT ratio of DSI compared with normal group. After treatment with velusetrag 3 mg/kg, p-mTOR, p-AKT, p-P70S6, mTOR and AKT proteins all decreased significantly.

The protein level of p-Akt, Akt, p-P70S6, p-mTOR and mTOR of DSI increase in PrP-SCA7-92Q transgenic vehicle group in which it showed significant difference on p-Akt, p-P70S6 and Akt compared with normal group. The ratio of p-Akt/AKT of DSI increased significantly in vehicle group. Treatment with velusetrag, 3 mg/kg reduced the p-Akt, Akt, p-P70S6, p-mTOR and mTOR protein level in which it showed significant difference on p-Akt, Akt and p-P70S6. Treatment with velusetrag 1 mg/kg reduced the ratio of p-mTOR/mTOR significantly.

The number of calretinin, Hu, nNOS and CHAT stained neurons decreased in DSI and colon of PrP-SCA7-92Q transgenic mice. After treatment of velusetrag 1 mg/kg and 3 mg/kg, the number of calretinin, Hu, nNOS and CHAT stained neurons increased significantly in DSI and/or colon.

The unexpected experimental results further show that both dosages of velusetrag, i.e. 1 mg/kg and 3 mg/kg were effective in improving weight loss, bowel dilatation and lethality, whereas concerning inflammation and ulceration the higher dose of 3 mg/kg was more effective, restoring the status of healthy animals. As regards the effects of velusetrag on the neuronal parameters, such as an increase in nitrergic neurons or an increase in cholinergic neurons, both dosages provide similar results. Hence, velusetrag is particularly effective as regards the treatment of neuropathic CIPO and additionally has a significant anti-inflammatory effect on the gut.

Overall, the results of the carried-out experimentation and herewith presented, have demonstrated that velusetrag provides modulatory, neuroprotective and neurotrophic effects towards the ENS in both the tested CIPO animal models. The improvement of enteric nervous system and the decrease of inflammation signs after treatment indicate the value of velusetrag on therapy of disorders of neuroenteric system correlated to intestinal dysmotility diseases.

In view of the demonstrated technical effect, the proposed use of velusetrag is for the treatment of Idiopathic Chronic Intestinal Pseudo-obstruction or secondary to neurodegeneration or being secondary to autoimmune conditions or secondary to connective tissue disorders or being secondary to demyelinating conditions affecting intestinal motility. The conditions which may benefit of this use are Idiopathic Chronic Intestinal Pseudo-obstruction or secondary to neurodegeneration or being secondary to autoimmune conditions or secondary to connective tissue disorders or being secondary to demyelinating conditions affecting intestinal motility through one or more of the following: diseases of the autonomic nervous system (i.e. stroke, encephalitis, calcification of basal ganglia, orthostatic hypotension); diseases of the intestinal wall nervous system (i.e. paraneoplastic syndrome, viral infections, iatrogenic disorders, Hirschsprung's disease, Chagas' disease, Von Recklinghausen's disease); diseases of the intestinal wall muscle layer (i.e. myotonic dystrophy, progressive systemic sclerosis); diseases of the mixed enteric nervous system and smooth muscle layer (scleroderma, dermatomyositis, amyloidosis, Ehler-Danlos syndrome); and by unknown mechanism (i.e. hypothyroidism, hypoparathyroidism, pheochromocytoma, antidepressants drugs, antineoplastics, bronchodilatators).

Due to the technical effect provided by the use of velusetrag syndromes caused or characterized by inflammatory/immune infiltrates of neurons located in submucosal and myenteric ganglia of the enteric nervous system by cellular infiltrates of circulating antineuronal antibodies, resulting in intestinal motility disorder may greatly benefit from the above treatment. The treatment of the invention is effective for the acute and chronic forms of the above forms of CIPO.

### Pharmaceutical Compositions and Formulations

The invention also relates to pharmaceutical compositions comprising velusetrag or a pharmaceutically acceptable salt thereof in the treatment of CIPO.

The term “pharmaceutically acceptable” refers to a material that is not biologically or otherwise unacceptable when used in the invention. For example, the term “pharmaceutically acceptable carrier” refers to a material that can be incorporated into a composition and administered to a patient without causing unacceptable biological effects or interacting in an unacceptable manner with other components of the composition. Such pharmaceutically acceptable materials typically have met the required standards of toxicological and manufacturing testing, and include those materials identified as suitable inactive ingredients by the U.S. Food and Drug administration.

The term “pharmaceutically acceptable salt” means a salt prepared from a base or an acid which is acceptable for administration to a patient, such as a mammal (for example, salts having acceptable mammalian safety for a given dosage regime). Pharmaceutically acceptable salts can be derived from pharmaceutically acceptable inorganic or organic bases and from pharmaceutically acceptable inorganic or organic acids. In addition, when a compound contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, 2-diethylamine, 2-diethylaminoethanol, dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (for example, citric, gluconic, glycolic, lactic, lactobionic, malic, and tartaric acids), aliphatic monocarboxylic acids (for example, acetic, butyric, formic, propionic and trifluoroacetic acids), amino acids (for example, aspartic and glutamic acids), aromatic carboxylic acids (for example, benzoic, p-chlorobenzoic, diphenylacetic, gentisic, hippuric, and triphenylacetic acids), aromatic hydroxyl acids (for example, o-hydroxybenzoic, p-hydroxybenzoic, 1-hydroxynaphthalene-2-carboxylic and 3-hydroxynaphthalene-2-carboxylic acids), ascorbic, dicarboxylic acids (for example, fumaric, maleic, oxalic and succinic acids), glucoronic, mandelic, mucic, nicotinic, orotic, pamoic, pantothenic, sulfonic acids (for example, benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like.

The term “treating” or “treatment” includes preventing, alleviating, ameliorating, giving relief to symptoms associated to CIPO, as assessed by a change in weekly global gastrointestinal symptoms average index score from start to the end of each treatment period.

The term “symptoms associated to CIPO” includes abdominal pain, bloating, nausea, vomiting.

According to the present invention a treating effect is present if at least one is observed:
change in waist circumference from start to the end of each treatment period,
1-point improvement in weekly global gastrointestinal symptoms average index score from start to the end of each treatment period,
change in individual symptoms score from start to the end of each treatment period for abdominal pain, bloating, nausea, vomiting,
change in number of weekly bowel movements from start to the end of each treatment period (only in subjects with Bristol scale type 1 or 2 at the start of the treatment period),
change in number of weekly complete evacuations from start to the end of each treatment period,
change in stool type in the Bristol stool scale from start to the end of each treatment period,
change in weekly bowel habit satisfaction score from start to the end of each treatment period measured using a scale from 0 to 10,
change in orocecal transit time measured using lactulose breath test from start at the end of the first treatment period,
proportion of number of days with change in medications used to relieve main CIPO gastrointestinal symptoms during each treatment period, wash-out period and follow-up period (proportion of days with dose increased compared or decreased compared to the start of the period, proportion of days with drug added or removed compared to the start of the period,
change in the quality of life (SF-12) from start to the end of each treatment period,
number of CIPO-related hospitalization during the treatment period,
change in the need of artificial food,
change in number of pseudo-obstruction episodes based on investigation's judgement from start to the end of each treatment period,
change from the end of each treatment period to the ends of the first and the second week of wash out, or follow-up period, in:
   weekly abdominal pain score,
   weekly bloating score,
   weekly nausea score,
   weekly vomiting score,
   weekly global gastrointestinal symptoms average index score,
   number of weekly bowel movements,
   number of weekly complete evacuations,
   stool type at the Bristol stool scale,
   weekly bowel habit satisfaction score measured using a scale from 0 to 10.

The SF-12 Health Survey is a shortened version of its predecessor, the SF-36, which itself evolved from the Medical Outcomes Study used for the international assessment of the patients quality of life [Gandek B. et al. Cross-Validation of Item Selection and Scoring for the SF-12 Health Survey in Nine Countries: Results from the IQOLA Project, Journal of Clinical Epidemiology, 1998; 51:1171-1178].

The Bristol Scale or Chart is a clinical assessment tool developed in 1997 designed to classify stools into seven groups. [Russo M. et al. Stool Consistency, but Not Frequency, Correlates with Total Gastrointestinal Transit Time in Children. The Journal of Pediatrics, 2013; 162:1188-1192]. This tool provides a classification of the patients' ejection based on shapes and types of faeces, that has good correlation with the time it takes for food to pass through the gastrointestinal tract and leave as waste.

The term “unit dosage form” refers to a physically discrete unit suitable for dosing a patient, i.e., each unit containing a predetermined quantity of active agent calculated to produce the desired therapeutic effect either alone or in combination with one or more additional units.

The crystalline hydrochloride salt forms of velusetrag are typically administered to a patient in the form of a pharmaceutical composition. Such pharmaceutical compositions may be administered to the patient by any acceptable route of administration including, but not limited to, oral, rectal, vaginal, nasal, inhaled, topical (including transdermal) and parenteral modes of administration. Accordingly, in one of its composition's aspects, the invention is directed to a pharmaceutical composition comprising a pharmaceutically-acceptable carrier or excipient and a therapeutically effective amount of a crystalline hydrochloride salt of a compound of Formula I. Optionally, such pharmaceutical compositions may contain other therapeutic and/or formulating agents if desired.

The pharmaceutical compositions of the invention typically contain a therapeutically effective amount of a crystalline salt of the present invention. Typically, such pharmaceutical compositions will contain from about 0.1 to about 95% by weight of the active agent, including from about 1 to about 70% by weight, such as from about 5 to about 60% by weight of the active agent.

Any conventional carrier or excipient may be used in the pharmaceutical compositions of the invention. The choice of a particular carrier or excipient, or combinations of carriers or excipients, will depend on the mode of administration being used to treat a particular patient or type of medical condition or disease state. In this regard, the preparation of a suitable pharmaceutical composition for a particular mode of administration is well within the scope of those skilled in the pharmaceutical arts. Additionally, the ingredients for such compositions are commercially available from, for example, Sigma, P.O. Box 14508, St. Louis, MO 63178. By way of further illustration, conventional formulation techniques are described in Remington: The Science and Practice of Pharmacy, 20^{th} Edition, Lippincott Williams & White, Baltimore, Maryland (2000); and H. C. Ansel et al., Pharmaceutical Dosage Forms and Drug Delivery Systems, 7^{th} Edition, Lippincott Williams & White, Baltimore, Maryland (1999).

Representative examples of materials which can serve as pharmaceutically acceptable carriers include, but are not limited to, the following: (1) sugars, such as lactose, glucose and sucrose; (2) starches, such as corn starch and potato starch; (3) cellulose, such as microcrystalline cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

The pharmaceutical compositions of the invention are typically prepared by thoroughly and intimately mixing or blending a compound of the invention with a pharmaceutically acceptable carrier and one or more optional ingredients. If necessary or desired, the resulting uniformly blended mixture can then be shaped or loaded into tablets, capsules, pills and the like using conventional procedures and equipment.

The pharmaceutical compositions of the invention are preferably packaged in a unit dosage form. For example, such unit dosage forms may be capsules, tablets, pills, and the like.

In a preferred embodiment, the pharmaceutical compositions of the invention are suitable for oral administration. Suitable pharmaceutical compositions for oral administration may be in the form of capsules, tablets, pills, lozenges, cachets, sachets, stick-packs, dragees, powders, granules; or as a solution or a suspension in an aqueous or non-aqueous liquid; or as an oil-in-water or water-in-oil liquid emulsion; or as an elixir or syrup; and the like; each containing a predetermined amount of a compound of the present invention as an active ingredient.

When intended for oral administration in a solid dosage form (i.e., as capsules, tablets, pills and the like), the pharmaceutical compositions of the invention will typically comprise a compound of the present invention as the active ingredient and one or more pharmaceutically acceptable carriers, such as sodium citrate or dicalcium phosphate. Optionally or alternatively, such solid dosage forms may also comprise: (1) fillers or extenders, such as starches, microcrystalline cellulose, lactose, sucrose, glucose, mannitol, and/or silicic acid; (2) binders, such as carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and/or sodium carbonate; (5) solution retarding agents, such as paraffin; (6) absorption accelerators, such as quaternary ammonium compounds; (7) wetting agents, such as cetyl alcohol and/or glycerol monostearate; (8) absorbents, such as kaolin and/or bentonite clay; (9) lubricants, such as talc, calcium stearate, magnesium stearate, solid polyethylene glycols, sodium lauryl sulfate, and/or mixtures thereof; (10) coloring agents; and (11) buffering agents.

Release agents, wetting agents, coating agents, sweetening, flavoring and perfuming agents, preservatives and antioxidants can also be present in the pharmaceutical compositions of the invention. Examples of pharmaceutically-acceptable antioxidants include: (1) water-soluble antioxidants, such as ascorbic acid, cysteine hydrochloride, sodium bisulfate, sodium metabisulfate sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like. Coating agents for tablets, capsules, pills and like, include those used for enteric coatings, such as cellulose acetate phthalate (CAP), polyvinyl acetate phthalate (PVAP), hydroxypropyl methylcellulose phthalate, methacrylic acid ester, cellulose acetate trimellitate (CAT), carboxymethyl ethyl cellulose (CMEC), hydroxypropyl methyl cellulose acetate succinate (HPMCAS), and the like.

If desired, the pharmaceutical compositions of the present invention may also be formulated to provide slow or controlled release of the active ingredient using, by way of example, hydroxypropyl methyl cellulose in varying proportions; or other polymer matrices, liposomes and/or microspheres.

In addition, the pharmaceutical compositions of the present invention may optionally contain opacifying agents and may be formulated so that they release the active ingredient only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Suitable liquid dosage forms for oral administration include, by way of illustration, pharmaceutically acceptable emulsions, microemulsions, solutions, suspensions, syrups and elixirs. Such liquid dosage forms typically comprise the active ingredient and an inert diluent, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (esp., cottonseed, groundnut, corn, germ, olive, castor and sesame oils), glycerol, tetrahydrofuryl alcohol, polyethylene glycols and fatty acid esters of sorbitan, and mixtures thereof. Suspensions, in addition to the active ingredient, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof. Alternatively, the pharmaceutical compositions of the invention are formulated for administration by inhalation. Suitable pharmaceutical compositions for administration by inhalation will typically be in the form of an aerosol or a powder. Such compositions are generally administered using well-known delivery devices, such as a metered-dose inhaler, a dry powder inhaler, a nebulizer or a similar delivery device.

When administered by inhalation using a pressurized container, the pharmaceutical compositions of the invention will typically comprise the active ingredient and a suitable propellant, such as dichlorodifluoromethane, trichlorofluoromethane, dichlorotetrafluoroethane, carbon dioxide or other suitable gas.

Additionally, the pharmaceutical composition may be in the form of a capsule or cartridge (made, for example, from gelatin) comprising a compound of the invention and a powder suitable for use in a powder inhaler. Suitable powder bases include, by way of example, lactose or starch.

The compounds of the invention can also be administered transdermally using known transdermal delivery systems and excipients. For example, a compound of the invention can be admixed with permeation enhancers, such as propylene glycol, polyethylene glycol monolaurate, azacycloalkan-2-ones and the like, and incorporated into a patch or similar delivery system. Additional excipients including gelling agents, emulsifiers and buffers, may be used in such transdermal compositions if desired.

The following formulations illustrate representative pharmaceutical compositions of the present invention:

### Formulation Example A

Hard gelatin capsules for oral administration are prepared as follows:

| Ingredients | Amount |
|---|---|
| Velusetrag salt | 50 mg |
| Lactose (spray-dried) | 200 mg |
| Magnesium stearate | 10 mg |

Representative Procedure: The ingredients are thoroughly blended and then loaded into a hard gelatin capsule (260 mg of composition per capsule).

### Formulation Example B

Hard gelatin capsules for oral administration are prepared as follows:

| Ingredients | Amount |
|---|---|
| Velusetrag salt | 20 mg |
| Starch | 89 mg |
| Microcrystalline cellulose | 89 mg |
| Magnesium stearate | 2 mg |

Representative Procedure: The ingredients are thoroughly blended and then passed through a No. 45 mesh U.S. sieve and loaded into a hard gelatin capsule (200 mg of composition per capsule).

### Formulation Example C

Capsules for oral administration are prepared as follows:

| Ingredients | Amount | |
|---|---|---|
| Velusetrag salt | 10 | mg |
| Polyoxyethylene sorbitan monooleate | 50 | mg |
| Starch powder | 250 | mg |

Representative Procedure: The ingredients are thoroughly blended and then loaded into a gelatin capsule (310 mg of composition per capsule).

### Formulation Example D

Tablets for oral administration are prepared as follows:

| Ingredients | Amount | |
|---|---|---|
| Velusetrag salt | 5 | mg |
| Starch | 50 | mg |
| Microcrystalline cellulose | 35 | mg |
| Polyvinylpyrrolidone (10 wt. % in water) | 4 | mg |
| Sodium carboxymethyl starch | 4.5 | mg |
| Magnesium stearate | 0.5 | mg |
| Talc | 1 | mg |

Representative Procedure: The active ingredient, starch and cellulose are passed through a No. 45 mesh U.S. sieve and mixed thoroughly. The solution of polyvinylpyrrolidone is mixed with the resulting powders, and this mixture is then passed through a No. 14 mesh U.S. sieve. The granules so produced are dried at 50-60° C. and passed through a No. 18 mesh U.S. sieve. The sodium carboxymethyl starch, magnesium stearate and talc (previously passed through a No. 60 mesh U.S. sieve) are then added to the granules. After mixing, the mixture is compressed on a tablet machine to afford a tablet weighing 100 mg.

### Formulation Example E

Tablets for oral administration are prepared as follows:

| Ingredients | Amount | |
|---|---|---|
| Velusetrag salt | 25 | mg |
| Microcrystalline cellulose | 400 | mg |
| Silicon dioxide fumed | 10 | mg |
| Stearic acid | 5 | mg |

Representative Procedure: The ingredients are thoroughly blended and then compressed to form tablets (440 mg of composition per tablet).

### Formulation Example E

Single-scored tablets for oral administration are prepared as follows:

| Ingredients | Amount | |
|---|---|---|
| Velusetrag salt | 15 | mg |
| Cornstarch | 50 | mg |
| Croscarmellose sodium | 25 | mg |
| Lactose | 120 | mg |
| Magnesium stearate | 5 | mg |

Representative Procedure: The ingredients are thoroughly blended and compressed to form a single-scored tablet (215 mg of composition per tablet).

### Formulation Example G

A suspension for oral administration is prepared as follows:

| Ingredients | Amount | |
|---|---|---|
| Velusetrag salt | 0.1 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum k (Vanderbilt Co.) | 1.0 | g |
| Flavoring | 0.035 | mL |
| Colorings | 0.5 | mg |

| | |
|---|---|
| Distilled water | q.s. to 100 mL |

Representative Procedure: The ingredients are mixed to form a suspension containing 10 mg of active ingredient per 10 mL of suspension.

### Formulation Example H

A dry powder for administration by inhalation is prepared as follows:

| Ingredients | Amount | |
|---|---|---|
| Velusetrag salt | 1.0 | mg |
| Lactose | 25 | mg |

Representative Procedure: The active ingredient is micronized and then blended with lactose. This blended mixture is then loaded into a gelatin inhalation cartridge. The contents of the cartridge are administered using a powder inhaler.

### Formulation Example I

A dry powder for administration by inhalation in a metered dose inhaler is prepared as follows:

Representative Procedure: A suspension containing 5 wt. % of a salt of the invention and 0.1 wt. % lecithin is prepared by dispersing 10 g of active compound as micronized particles with mean size less than 10 μm in a solution formed from 0.2 g of lecithin dissolved in 200 mL of demineralized water. The suspension is spray dried and the resulting material is micronized to particles having a mean diameter less than 1.5 μm. The particles are loaded into cartridges with pressurized 1,1,1,2-tetrafluoroethane.

### Formulation Example I

An injectable formulation is prepared as follows:

| Ingredients | Amount | |
|---|---|---|
| Velusetrag salt | 0.2 | g |
| Sodium acetate buffer solution (0.4M) | 40 | mL |

| | |
|---|---|
| HCl (0.5N) or NaOH (0.5N) | q.s. to pH 4 |
| Water (distilled, sterile) | q.s. to 200 mL |

Representative Procedure: The above ingredients are blended, and the pH is adjusted to 4±0.5 using 0.5 N HCl or 0.5 N NaOH.

### Formulation Example K

Capsules for oral administration are prepared as follows:

| Ingredients | Amount | |
|---|---|---|
| Velusetrag salt | 4.05 | mg |
| Microcrystalline cellulose (Avicel PH 103) | 259.2 | mg |
| Magnesium stearate | 0.75 | mg |

Representative Procedure: The ingredients are thoroughly blended and then loaded into a gelatin capsule (Size #1, White, Opaque) (264 mg of composition per capsule).

### Formulation Example L

Capsules for oral administration are prepared as follows:

| Ingredients | Amount | |
|---|---|---|
| Velusetrag salt | 8.2 | mg |
| Microcrystalline cellulose (Avicel PH 103) | 139.05 | mg |
| Magnesium stearate | 0.75 | mg |

Representative Procedure: The ingredients are thoroughly blended and then loaded into a gelatin capsule (Size #1, White, Opaque) (148 mg of composition per capsule).

### Routes of Administration

The invention also relates to an acceptable route of administration of velusetrag to the human patient, including, but not limited to, oral, parenteral, buccal, sublingual, rectal, intraperitoneal, or endotracheal routes of administration. For example, parenteral administration may be by infusion, injection, or implantation. Parenteral may also include percutaneous administration via subcutaneous, intramuscular, intravenous, transdermal, or by implantation routes. If velusetrag is administered parenterally, it may be in the form of a liquid, solid or gel. Similarly, if velusetrag is administered orally, it may be in the form of a liquid, capsule, tablet, chewable tablet or dissolvable film.

The following examples are illustrative in purpose without limiting the scope of the invention described herein.

### EXAMPLES

### Animal Models

Rb1 cKO retinoblastoma mice [Fu M, et al. Retinoblastoma protein prevents enteric nervous system defects and intestinal pseudo-obstruction. J Clin Invest. 2013; 123:5152-5164] constitutes a model of intestinal pseudo-obstruction, in particular with neuronal dysfunction. In this model, velusetrag dissolved in saline was administered intra-peritoneally (ip) once daily at one of the two dosages (1 mg/10 ml/kg or 3 mg/10 ml/kg, n=10 animals/group) for 14 days starting at 15 days after birth. PrP-SCA7-92Q are as described in La Spada A R et al., Polyglutamine-expanded ataxin-7 antagonizes CRX function and induces cone-rod dystrophy in a mouse model of SCA7. Neuron 2001; 31:913-27. PrP-SCA7-92Q mice represent a model of human intestinal pseudo-obstruction, specifically with a visceral neuropathy with intranuclear neuronal inclusions and intestinal pseudo-obstruction. In this model, velusetrag was administered orally once daily at one of the two dosages (1 mg/10 ml/kg or 3 mg/10 ml/kg, n=10 animals/group) for 5 weeks starting at 8 weeks after birth.

As a positive control the modified mouse was administered vehicle (saline). As a negative control unmodified mouse was used.

### Statistical Analysis

All the data in Tables 1 to 17 were means±SEM of 10 mice per group. Statistical comparison was performed by ANOVA (Graph Pad Prism 9.3) where, P value vs. Normal was defined with the symbol * and P value vs. cKO vehicle was mentioned with the symbol ^{∘}. In particular, *P<0.05, **p<0.01, ***p<0.001, ****P<0.0001 vs. normal; ^{∘}p<0.05, ^{∘∘}P<0.01, ^{∘∘∘}P<0.001, ^{∘∘∘∘}P<0.0001, vs. cKO+ vehicle.

### Example 1—Effect of Velusetrag on the Lethality of Mice of Animal Models

C57BL/6 (Cyagen, China) and Rb1 cKO retinoblastoma mice (Cyagen, China) with free access to water and food were used. Velusetrag was administered once daily at one of the two doses (3 mg/10 mL/kg or 1 mg/10 mL/kg) ip for 14 days starting 15 days after birth. 10 mice were monitored only for survival up to 60 days after birth. As reported in Table 1, 40% of the control group (saline-treated KO animals) died within 15 days after the start of treatment. By contrast, only 10% of knock out mice treated with velusetrag at 1 or 3 mg/kg died. Then, velusetrag has a significant benefit on death rate.

Further, at both dosages velusetrag is well tolerated as treated animals showed no weight loss (Table 1).

TABLE 1
Effect of velusetrag on body weight
and lethality in Rb1 knockout animals

| | Body weight | |
|---|---|---|
| | loss from | |
| | initial weight | |
| | of 7 g at | |
| Group | 15 days % | Lethality |
| C57BL/6 (Normal) administrated with | 0 | 0/10 |
| vehicle (sterile saline solution) | | |
| Rb1cKO + Vehicle (sterile saline solution) | 0 | 4/10 |
| Rb1cKO + Velusetrag 1 mg/10 mL/kg ip | 0 | 1/10 |
| Rb1cKO + Velusetrag 3 mg/10 mL/kg ip | 0 | 1/10 |

Similar results were observed in Prp-SCA7-92Q mice.

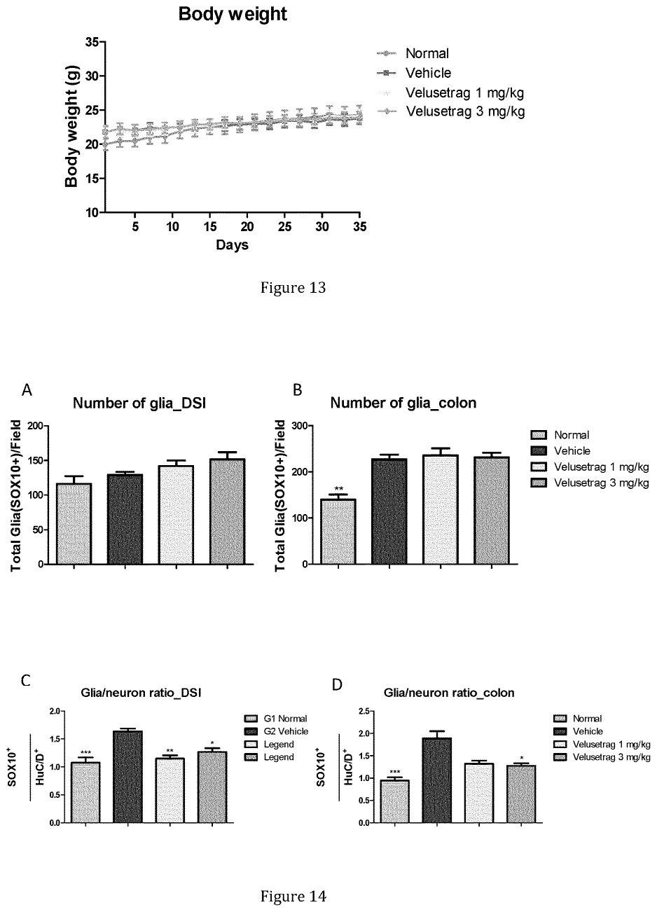

To assess if velusetrag was well-tolerated in this mouse model, the body weight of Prp-SCA7-92Q mice treated either with vehicle or with the 1 or 3 mg/kg velusetrag was measured throughout the treatment. A normal control (NC) group of untreated wild-type mice was also examined. No significant differences were noticed between groups (FIG. 13).

After 5 weeks of treatment, the body weight of transgenic mice did not show differences compared with the normal control group. The Velusetrag treatment at 1 and 3 mg/kg was well-tolerated, no reduction in body weight and lethality was observed throughout treatment (FIG. 13).

### Example 2—Effect of Velusetrag on Intestinal Dilatation

C57BL/6 and Rb1 cKO retinoblastoma mice (Cyagen, China) with free access to water and food were used. Velusetrag was administered once daily at two doses (3 mg/10 mL/kg or 1 mg/10 mL/kg) ip for 14 days starting after 15 days after birth until day 28.

C57BL/6 mice (Cyagen, China) and Prp-SCA7-92Q transgenic mice with free access to water and food administered were administrated with velusetrag once daily at two doses (3 mg/10 mL/kg and 1 mg/10 mL/kg) by the oral route for 5 weeks starting after 8 weeks of birth until 13 weeks of age.

The following day of the last treatment animals were sacrificed by CO₂ inhalation. Distal small intestine (DSI) and proximal colon samples were collected and washed with PBS. The diameters of DSI and colon were measured by Image J (version 1.53c) based on a scaled plate where the tissues were placed when images were taken. Diameters of distal small intestine (DSI) and proximal colon in five regions were evaluated and expressed in mm.

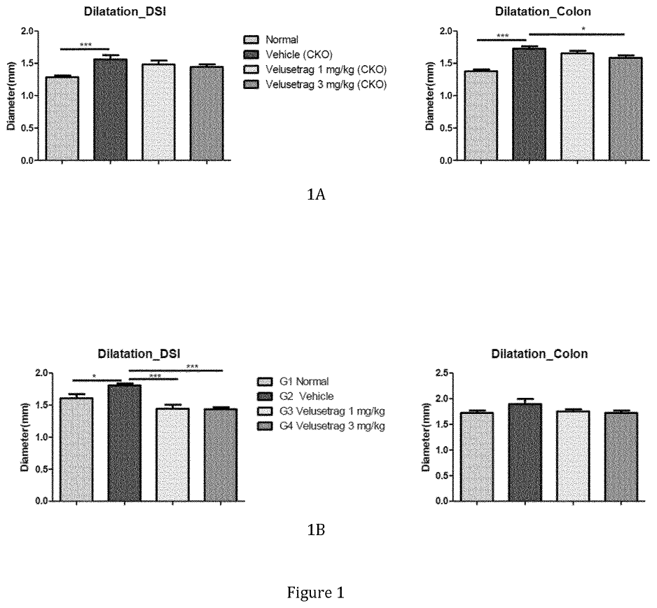

Rb1 cKO mice show an intestinal dilation with an increment in both DSI and proximal colon diameters when compared to wild-type, normal animals. In Rb1 cKO mice after administration of velusetrag at both tested doses there is a reduction in intestinal dilatation in both the distal small intestine and proximal colon compared to the untreated KO mouse. The reduction is dose dependent with a higher effect at the higher dose of velusetrag (FIG. 1A and Table 2).

TABLE 2
Effect of velusetrag on distal small intestine (DSI) and
proximal colon diameter expressed in mm in Rb1 cKO mice.

| | | Prox |
|---|---|---|
| Group | DSI (mm) | Colon (mm) |
| C57BL/6 (Normal) administrated | 1.29 ± 0.01 | 1.38 ± 0.01 |
| with vehicle (sterile | | |
| saline solution) | | |
| Rb1cKO + Vehicle | 1.56 ± 0.02*** | 1.73 ± 0.01**** |
| (sterile saline solution) | | |
| Rb1cKO + velusetrag | 1.48 ± 0.02** | 1.65 ± 0.01**** |
| 1 mg/10 mL/kg ip | | |
| Rb1cKO + velusetrag | 1.44 ± 0.01** | 1.58 ± 0.01***^{∘} |
| 3 mg/10 mL/kg ip | | |

Prp-SCA7-92Q transgenic mice also show an intestinal dilation with an increment in both DSI and proximal colon diameters when compared to wild-type, normal animals. In Prp-SCA7-92Q transgenic mice velusetrag has shown to significantly reduce dilatation of the distal small intestine at both doses tested in both the distal small intestine and proximal colon compared to the vehicle-treated transgenic mouse. In the proximal colon, following treatment with 3 mg/10 ml/kg velusetrag, intestinal dilatation reverts to values of the healthy animal (Table 3 and FIG. 1B).

TABLE 3
Effect of velusetrag on bowel dilatation
in Prp-SCA7-92Q transgenic mice.

| | | Prox |
|---|---|---|
| Group | DSI (mm) | colon (mm) |
| C57BL/6 (Normal) administrated with | 1.61 ± | 1.72 ± |
| vehicle (sterile saline solution) | 0.2 | 0.2 |
| Prp-SCA7-92Q + Vehicle | 1.81 ± | 1.9 ± |
| | 0.01* | 0.03 |
| Prp-SCA7-92Q + velusetrag 1 mg/kg po | 1.44 ± | 1.75 ± |
| | 0.02^{∘∘∘∘} | 0.01 |
| Prp-SCA7-92Q + velusetrag 3 mg/kg po | 1.44 ± | 1.72 ± |
| | 0.01^{∘∘∘∘} | 0.01 |

Velusetrag at both doses of 1 and 3 mg/kg groups was able to significantly counteract dilatation, by normalizing the dilatation of DSI at both doses (FIG. 1B).

### Example 3—Effect of Velusetrag on Intestinal Morphometry (Anti-Inflammatory Activity)

After mice were sacrificed by CO₂ inhalation, the small intestine and colon tissue were cut, fixed for 48 hours with 10% neutral buffered formalin. The paraffin sample was prepared by dehydration with a series of gradient alcohol solutions according to standard methods known in the field. Tissues were cut into 4 μm sections by rotary microtome and baked for 2 hours at 60° C. After (HE) hematoxylin-eosin (HE) images were obtained from each animal at 20× magnification for histological analyses by fully automatic digital pathology slide-system (KF BIO, KF-PRO-005, China) using K-viewer Imaging software (KF BiO, V1, China). Ulcer (statistical area of intestinal mucosal damage), inflammation and bleeding of pathological features were evaluated, and scoring was attributed according to the table below.

Histological analysis scores

| Pathological changes | Pathological characteristics | score |
|---|---|---|
| Ulcers (significant | None | 0 |
| area of intestinal | 0%-5% | 1 |
| mucosal damage) | 5%-33% | 2 |
| | >33% | 3 |
| Inflammation | None | 0 |
| (20X microscopic | <2 | 1 |
| necrosis count) | 2-4 | 2 |
| | >4 | 3 |
| Bleeding | None | 0 |
| | Small bleeding area | 1 |
| | Large bleeding area | 2 |

The anti-inflammatory activity of velusetrag was evaluated by histological analysis of intestine samples isolated from mice of the two models. The presence of inflammation and ulcers was quantified using indicators (i.e. ulcers=% area with intestinal mucosal damage, inflammation=necrosis, bleeding) to which a score from 0 to 3 was associated. Inflammation and ulcers scoring were awarded after section evaluation and analysis.

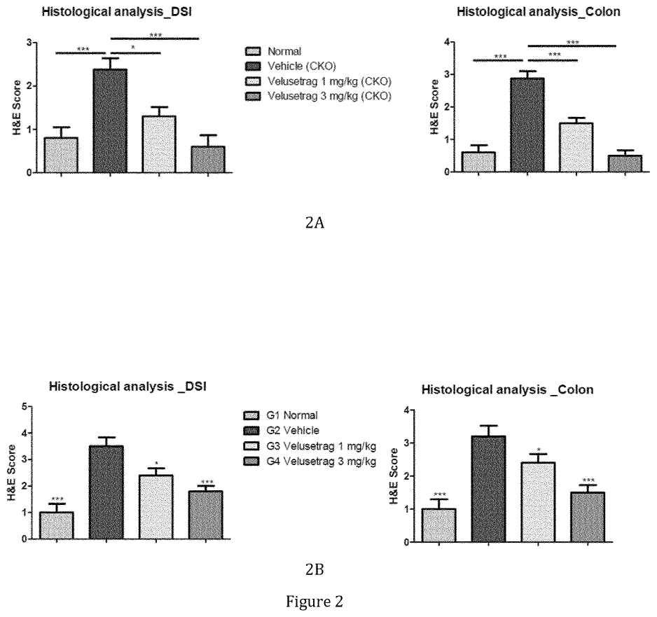

Rb1 cKO mice show an increased value of all measured parameters in both DSI and proximal colon diameters when compared to wild-type, normal animals. In Rb1 cKO mice both doses of velusetrag determined a reduction of both inflammation and ulceration compared to the untreated KO animal (positive control). Treatment with 3 mg/10 ml/kg velusetrag is able to restore the parameters to the values of the healthy animal (WT, normal mouse) (negative control) in both the distal small intestine and proximal colon (Table 4 and FIG. 2A).

TABLE 4
Effect of velusetrag treatment on morphometric gut evaluation in Rb1 cKO mice.

| | Inflammation | Ulcers | | Inflammation | Ulcers | |
|---|---|---|---|---|---|---|
| | (count of | (mucosal | Total | (count of | (mucosal | Total |
| | necrosis) | damage) | score | necrosis) | damage) | score |
| Group | DSI | | | Proximal Colon | | |
| C57BL/6 + | 0.6 ± 0.05 | 0.2 ± 0.04 | 0.8 ± 0.1 | 0.5 ± 0.05 | 0.1 ± 0.03 | 0.6 ± 0.07 |
| vehicle (sterile | | | | | | |
| saline solution) | | | | | | |
| Rb1cKO + | 1.63 ± 0.06*** | 0.75 ± 0.06* | 2.38 ± 0.09*** | 1.88 ± 0.04**** | 1.0 ± 0.09*** | 2.88 ± 0.08**** |
| Vehicle (sterile | | | | | | |
| saline solution) | | | | | | |
| Rb1cKO + | 0.9 ± 0.03^{∘∘} | 0.40 ± 0.05 | 1.3 ± 0.07^{∘∘} | 1.0 ± 0.01**^{∘∘∘∘} | 0.5 ± 0.05* | 1.5 ± 0.05**^{∘∘∘} |
| velusetrag | | | | | | |
| 1 mg/10 mL/kg | | | | | | |
| ip | | | | | | |
| Rb1cKO + | 0.4 ± 0.05^{∘∘∘} | 0.20 ± 0.04^{∘} | 0.6 ± 0.08^{∘∘∘} | 0.50 ± 0.05^{∘∘∘∘} | 0^{∘∘∘} | 0.5 ± 0.05^{∘∘∘∘} |
| velusetrag | | | | | | |
| 3 mg/10 mL/kg | | | | | | |
| ip | | | | | | |

Similarly, Prp-SCA7-92Q mice show an increased value of all measured parameters in both DSI and proximal colon diameters when compared to wild-type, normal animals In Prp-SCA7-92Q mice both doses of velusetrag determined a reduction of both inflammation and ulceration compared to the positive control, significantly improving the value compared to the positive control with the 3 mg/10 ml/kg dose of velusetrag in both the distal small intestine and proximal colon (Table 5 and FIG. 2B).

TABLE 5
Effect of velusetrag treatment on morphometric gut evaluation in Prp-SCA7-92Q transgenic mice

| | Inflammation | Ulcers | | Inflammation | Ulcers | |
|---|---|---|---|---|---|---|
| | (count of | (mucosal | Total | (count of | (mucosal | Total |
| | necrosis) | damage) | score | necrosis) | damage) | score |
| Group | DSI | | | Proximal Colon | | |
| C57BL/6 + | 0.6 ± 0.1 | 0.4 ± 0.1 | 1.0 ± 0.1 | 0.7 ± 0.1 | 0.2 ± 0.01 | 1.0 ± 0.1 |
| vehicle | | | | | | |
| Prp-SCA7-92Q + | 2.0 ± 0.1**** | 1.5 ± 0.1** | 3.5 ± 0.1**** | 2.0 ± 0.1**** | 1.2 ± 0.1**** | 3.2 ± 0.1**** |
| Vehicle | | | | | | |
| Prp-SCA7-92Q + | 1.6 ± 0.1*** | 0.8 ± 0.1^{∘} | 2.4 ± 0.1** | 1.5 ± 0.1*** | 0.9 ± 0.05** | 2.4 ± 0.1** |
| velusetrag | | | | | | |
| 1 mg/kg po | | | | | | |
| Prp-SCA7-92Q + | 1.2 ± 0.1*^{∘∘∘} | 0.6 ± 0.1^{∘} | 1.8 ± 0.1**^{∘} | 1.1 ± 0.1*^{∘∘∘} | 0.4 ± 0.01^{∘∘} | 1.5 ± 0.1*^{∘∘∘} |
| velusetrag | | | | | | |
| 3 mg/kg po | | | | | | |

Total scores for both models are also represented in FIGS. 2A and 2B. These findings showed that the CKO and Prp-SCA7-92Q transgenic mice display damage of the intestinal structural integrity, whereas administration of Velusetrag effectively ameliorated intestinal abnormalities in a dose-dependent fashion (P<0.05 and P<0.001, respectively for the two dosages).

### Example 4—Velusetrag Activity on the AKT/mTOR/p70s6k Signal Pathway

The effect of velusetrag administration on the activation of the PI3K/AKT/mTOR signaling pathway was assessed by Western blot on extracts of distal small intestine samples from treated animals using specific antibodies directed to the proteins: p-Akt, Akt, p-mTOR, m-TOR, p-P70S6, Actin.

Animals were sacrificed by CO₂ inhalation the next day of the last treatment. Distal small intestine samples were collected and placed in 10 volumes (w:v) of lysing buffer (Beyotime Biotechnology, China) containing protease and phosphatase inhibitor cocktail (Thermo Scientific, US) according to the manufacturer's instructions. The tissue samples were homogenized on ice for 15 sec, incubated on ice for 30 min, and then centrifuged at 15,000×g for 15 min. The supernatant was collected, and protein concentration was determined by BCA method.

Proteins were fractionated on 10% SDS-PAGE gels and transferred to NC membranes. After blocking with 5% BSA in TBST (0.05% Tween-20) for 2 hrs at RT, the membranes were incubated overnight (no less than 15 h) with the following primary antibodies: p-Akt 1:1000 (Cell Signaling Technology, 4060S, US), Akt 1:1000 (Cell Signaling Technology, 9272, US), p-mTOR 1:1000 (Cell Signaling Technology, 5536S, US), mTOR 1:1000 (Cell Signaling Technology, 2972, US), p-P70S6 1:1000 (Cell Signaling Technology, 9204, US), nNOS 1:1000 (Abcam, ab76067, US), ChAT 1:1000 (Millipore, AB144P, US), Actin 1:1000 (Beyotime, AF5003, China) or MAPK 1:1000 (Cell Signaling Technology, 4695, US) at 4° C. The blots were washed with TBST (3 times, 10 min each) and incubated with following secondary antibodies: Goat anti-Rabbit IgG H&L (IRDye® 800CW) preadsorbed 1:5000 (Abcam, 216773, US) or Donkey anti-Goat IgG H&L (IRDye® 800CW) 1:5000 (Abcam, 216775, US) for 1 hr at RT. After wash, the membranes were placed in substrate working solution for 5 min prior to imaging using ChemiDoc System (Bio-Rad, 12003154, US). Among these biomarkers, Akt and mTOR were re-probed after membrane stripping using Restore™ Western Blot Stripping Buffer (Thermo Fisher Scientific, 21059, US) according to manufacturer's protocol. Densitometric analysis of protein bands were performed with QuantityOne software version 4.6.2. In Rb1 knock-out mice treated with vehicle an increase in the level of pAKT, mTOR, p-mTOR and p70SKal expression is observed when compared to wild-type C57Bl/6 mice. Treatment with velusetrag at a dosage of 3 mg/10 ml/kg inhibits signal activation resulting in a reduction in the protein level of the AKT/mTOR/p70s6k pathway compared to the positive control. The 1 mg/kg dosage reduced protein expression compared to the positive control, but not significantly (Table 6).

TABLE 6
Velusetrag effect on AKT/mTOR signal pathway in DSI of RB1 cKO mice

| Group | AKT/Actin | pAKT/Actin | mTOR/Actin | pmTOR/Actin | P70S6K |
|---|---|---|---|---|---|
| C57BL/6 + | 1.0 ± 0.1 | 1.0 ± 0.1 | 1.0 ± 0.2 | 1.0 ± 0.2 | 1.0 ± 0.1 |
| vehicle (sterile | | | | | |
| saline solution) | | | | | |
| Rb1cKO + Vehicle | 1.0 ± 0.1 | 2.2 ± 0.3** | 1.7 ± 0.4 | 1.9 ± 0.4 | 1.7 ± 0.2* |
| (sterile saline | | | | | |
| solution) | | | | | |
| Rb1cKO + velusetrag | 0.92 ± 0.1 | 2.0 ± 0.3* | 1.5 ± 0.5 | 1.7 ± 0.5 | 1.6 ± 0.3 |
| 1 mg/10 mL/kg ip | | | | | |
| Rb1cKO + velusetrag | 0.6 ± 0.1^{∘} | 1.3 ± 0.3^{∘} | 0.6 ± 0.3^{∘} | 0.6 ± 0.3^{∘} | 0.9 ± 0.2^{∘} |
| 3 mg/10 mL/kg ip | | | | | |

In Prp-SCA7-92Q transgenic mice treated with vehicle an increase in the level of pAKT, mTOR, p-mTOR and p70SKal expression is observed when compared to wild-type C57Bl/6 mice. In this model, the highest dose of velusetrag results in a reduction of the protein level of the AKT/mTOR/p70s6k pathway compared to the positive control (Prp-SCA7-92Q transgenic mice treated with vehicle). The 1 mg/Kg dosage does not seem to have any effect, as similar or in some cases even higher values were found compared to the positive control (Table 7).

TABLE 7
Velusetrag effect on AKT/mTOR signal pathway on DSI in Prp-SCA7-92Q transgenic mice

| Group | AKT/Actin | pAKT/Actin | mTOR/Actin | pmTOR/Actin | P70S6K |
|---|---|---|---|---|---|
| C57BL/6 + | 1.0 ± 0.3 | 1.0 ± 0.2 | 1.0 ± 0.7 | 1.0 ± 0.7 | 1.0 ± 0.4 |
| vehicle (sterile | | | | | |
| saline | | | | | |
| solution) | | | | | |
| Prp-SCA7-92Q + | 3.5 ± 0.3*** | 6.2 ± 1.0**** | 2.8 ± 1.3 | 4.1 ± 1.9 | 6.7 ± 2.0* |
| Vehicle | | | | | |
| Prp-SCA7-92Q + | 3.0 ± 0.6** | 6.4 ± 1.7**** | 6.4 ± 2.0* | 7.5 ± 2.4* | 3.4 ± 1.0* |
| velusetrag | | | | | |
| 1 mg/kg po | | | | | |
| Prp-SCA7-92Q + | 2.0 ± 0.5 | 3.6 ± 1.3 | 1.7 ± 0 | 2.4 ± 1.2 | 1.5 ± 0.5^{∘} |
| velusetrag | | | | | |
| 3 mg/kg po | | | | | |

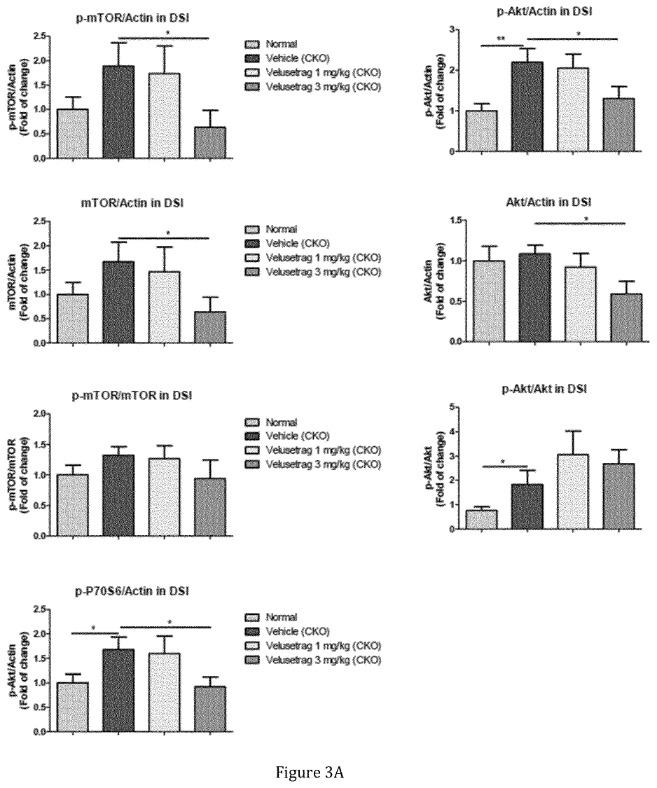

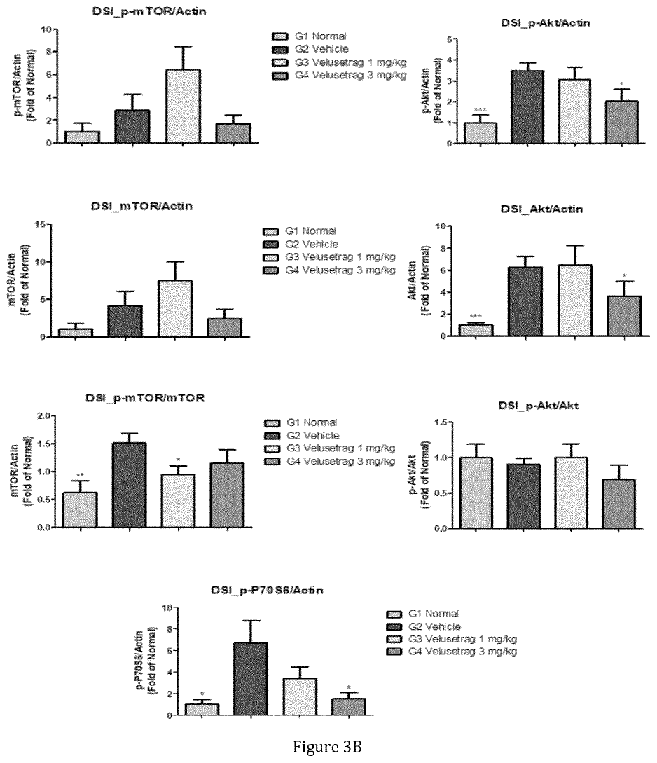

These results are also shown in FIG. 3A (Rb1cKO mice model) and 3B (PrP-SCA7-92Q transgenic mice model).

### Example 5—Effect of Velusetrag on MAP2 Dendridic Intensity

Microtubule-associated protein 2 (MAP2)-immunostaining chiefly visualizes the perikaryal-dendritic domain and the proximal part of the axonal processes in the enteric neurons of the porcine gut, hence it enables the unambiguous immunocytochemical identification of enteric multi (short) dendritic uniaxonal type I neurons. The effect of velusetrag treatment on dendrites of enteric neurons was evaluated on wholemount specimens isolated from animals of the two CIPO models treated as above described.

Mice were sacrificed with CO₂ inhalation the day after the last treatment. Samples of distal small intestine (DSI) and proximal colon were collected and washed in PBS to be subsequently processed for immunohistochemistry.

The tissues were cut along the mesenteric edge and they were pinned at a side on Sylgard plates. After the fixing with 4% paraformaldehyde for 30 min, the muscle layers were separated by mucosa and submucosa by using a fine-tipped forceps under the dissecting microscope.

The samples of DSI and colon were cut in segments of 1 cm and kept in 50% glycerol/PBS at −20° C. until the staining and the analysis. The preparations of the myenteric plexus were washed with PBST (PBS+0.5% Triton X 100) for 3 times and processed as follows:

Wholemount tissue was washed with PBST (PBS+0.5% Triton X-100) for 3 times. Tissue was incubated with stripping buffer for 40 minutes at 37° C.

Wholemount tissue was washed with PBST (PBS+0.5% TritonX-100) for 3 times.

Tissue was incubated with primary antibody MAP2 1:1000 (Abcam, US) overnight at 4° C.

Tissue was washed with TBST (PBS+0.5% Tritonx-100+0.5% Tween-20) for 3 times.

Tissue was incubated with second antibody F(ab′)2-Goat anti-Rabbit HRP 1:1000 (Abcam, US) for 1 h at room temperature.

Tissue was washed with PBST (PBS+0.5% Triton X-100) for 3 times.

Tissue was incubated with TSA670 1:200 (Wi See Biotechnology, China) for 1 h at room temperature from light.

Tissue was washed with PBST (PBS+0.5% Triton X-100) for 3 times.

Tissue was incubated with DAPI 1:5000 (Invitrogen, US) for 10 minutes at room temperature.

The coverslip was mounted with a drop of ProLong™ Glass Antifade Mountant (Invitrogen, US).

Tissue specimens were excited by laser with excitation and barrier filters set for individual fluorophores according to their specific excitation/emission spectra. Images were obtained from each animal using confocal scanning microscope (Operetta CLS high Content Analysis System, PerkinElmer, US) with a water immersion ×20 objective for cell counting. MAP2+ cells were counted.

Results are presented in Table 8.

TABLE 8
Effect of velusetrag MAP2 dendritic intensity in Rb1 cKO mice.

| | Total | |
|---|---|---|
| | (MAP2+) neurons/field | |
| | (0.26 mm²) | |
| | | Proximal |
| Group | DSI | colon |
| C57BL/6 + vehicle (sterile saline solution) | 72.8 ± 2.1 | 111.3 ± 3.2 |
| Rb1cKO + vehicle (sterile saline solution) | 44.6 ± 2.9* | 57.8 ± 4.4** |
| Rb1cKO + velusetrag 1 mg/10 mL/kg ip | 72.3 ± 2.3^{∘} | 93.8 ± .4^{∘} |
| Rb1cKO + velusetrag 3 mg/10 mL/kg ip | 81.4 ± 2.7^{∘∘} | 79.8 ± 4.2 |

In Rb1 cKO mice the number of MAP2 positive neurons in DSI and proximal colon was significantly decreased (DSI P<0.05; colon P<0.01) when compared to the wild-type group. The number of MAP2 positive neurons in DSI and proximal colon of Rb1 cKO mice treated with velusetrag, either at dose of 1 mg/kg or 3 mg/kg, was significantly increased compared to vehicle-treated cKO animals (DSI: 1 mg/kg group, P<0.05; 3 mg/kg group, P<0.01; colon: 1 mg/kg group, P<0.05). Then, in DSI, velusetrag administration reverts the reduction in MAP2 positive neurons to a level comparable to wild-type animal. The effect is slightly lower in the proximal colon (Table 8).

The same immunostaining experiment was carried out on intestinal wholemount specimens isolated from Prp-SCA7-92Q transgenic mice. Results show a decrease in dendrites in transgenic animals compared to wild type (control) animals (Table 9).

TABLE 9
Effect of velusetrag on MAP2 dendritic intensity
of transgenic mice Prp-SCA7-92Q

| | Total Neurouns | |
|---|---|---|
| | (MAP2+)/Field | |
| | (0.26 mm2) | |
| | | Proximal |
| Group | DSI | colon |
| C57BL/6 (Normal) administrated with | 96.1 ± 3.4 | 169.9 ± 4.2 |
| vehicle (sterile saline solution) | | |
| Prp-SCA7-92Q + Vehicle | 57.2 ± 1.1** | 134.2 ± 4.8 |
| Prp-SCA7-92Q + velusetrag 1 mg/kg po | 69 ± 1.8* | 160 ± 3.0 |
| Prp-SCA7-92Q + velusetrag 3 mg/kg po | 64.55 ± 1.5* | 157 ± 4.0 |

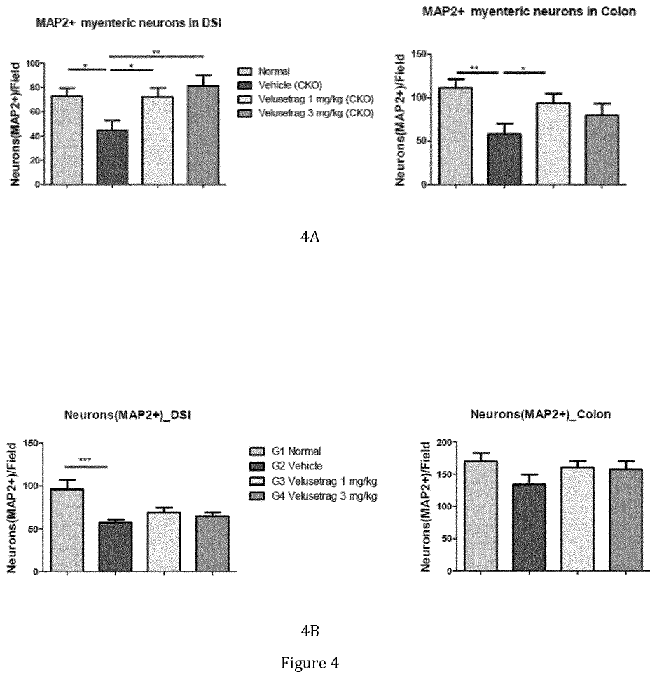

Rearrangement of the enteric neuronal network in PrP-SCA7-92Q mice treated only with vehicle was also visible by immunostaining of the myenteric plexus with MAP2, a cytoskeletal regulator within neuronal dendrites involved in neurite outgrowth, synaptic plasticity, and regulation of protein folding/transport as a robust somatodendritic marker (FIG. 4B). The number of MAP2+ neurons of DSI in transgenic mice treated with vehicle was significantly decreased compared to wild-type controls (P<0.001) only in DSI but not the proximal colon, suggesting a site-specific somatodendritic depletion. Velusetrag at both doses of 1 mg and 3 mg/kg had minor effects on the MAP2 staining (FIG. 4B).

FIGS. 4A and 4B show the effect of velusetrag on the number of MAP2+ neurons in DSI and colon proximal in murine model animals. 4A: Rb1cKO mice (*P<0.05, **P<0.01; T-Test; vs vehicle CKO). 4B: PrP-SCA7-92Q transgenic mice (***P<0.001; One way ANOVA; vs G2).

### Example 6—Effect of Velusetrag on Neuronal and Glia Cells by Whole-Mount Immunostaining

The effect of velusetrag on neuronal and glia cells in Rb1 retinoblastoma knock out mice was assessed using immunofluorescent analysis on distal small intestine and proximal colon samples with antibody directed to the glial marker SOX10, and the neuronal protein HuCD.

Animals were sacrificed by CO₂ inhalation the next day of the last treatment. Segments of distal ileum and colon were placed in phosphate-buffered saline and the mucosa and submucosa were manually removed with fine forceps. The muscularis propria and enclosed myenteric plexus were fixed 10 minutes in ice-cold acetone and then immersed in 1×PBS before blocking the tissues in 1.5% BSA blocking buffer and 1% Triton X-100 in PBS for 2 hours at room temperature. Samples were incubated with primary antibody against the glial protein SOX10 (Invitrogen, US) (1:250) overnight at +4° C.; following incubated with F(ab′)-goat anti-rabbit HRP secondary antibody (Abcam, US) (1:1000) for one hour at room temperature; washing were performed with PBST (PBS+0.5% Triton X-100) for 3 times; the incubation with fluorescent dye TSA520 (Wi See Biotechnology, China) (1:200) for 1 hour at room temperature, avoiding direct light; and following washing with PBST (PBS+0.5% Triton X-100) for 3 times. Detection of the neuronal marker HuCD was performed with the primary antibody HuCD (Abcam, US) (1:500) for 1 hour at room temperature; washed with TBST (PBS+0.5% Triton X-100+0.5% Tween 20) for 3 times, incubated with secondary antibody F(ab′)-goat anti-rabbit HRP (1:1000) for 1 hour at room temperature; washed with PBST (PBS+0.5% Triton X-100) for 3 times; incubated with fluorescent dye TSA570 (Wi See Biotechnology, China) (1:200) for 1 hour at room temperature, avoiding direct light; washed with PBST (PBS+0.5% Triton X-100) for 3 times; incubated with DAPI (1:5000) for 10 minutes at room temperature. Images were captured with microscope (Olympus, BX53). Tissue samples were excited with excitation lamp (UW, BWA and GW) and excitation/emission spectra (340-390 nm/420IF; 460-495 nm/510-550 nm; 530-550 nm/575IF). SOX10+ and HuCD+ antibodies combined with fluorescent dye TSA520 and TSA570 with specific excitation/emission spectra 488/519 nm and 555/570 nm. SOX10+ (green color) and HuCD+ (red color) cells were counted and the number of SOX10+ and HUCD+ neurons per mm² was evaluated.

The immunofluorescent analysis of the segments of distal ileum and colon has shown that velusetrag reduced glia cells (SOX10+ cells) and glia/neuron ratio. Thus, it decreases inflammation and gastrointestinal dysfunction. Detection of both glial and neuronal markers showed that at both doses of velusetrag there is a reduction in the glial marker signal (SOX10) and the glia/neuron ratio (SOX10/HuCD) in both the distal small intestine and proximal colon compared to untreated knock-out animals, however the reduction is more noticeable with the administration of the higher dose of velusetrag in both intestinal regions (Table 10).

TABLE 10
Effect of velusetrag on glia and glia/neuron ratio

| | SOX10+ | HuCD+ | SOX10+/ | SOX10+ | HuCD+ | SOX10+/ |
|---|---|---|---|---|---|---|
| | mm² | mm² | HuCD+ | mm² | mm² | HuCD+ |
| Group | DSI | | | Proximal colon | | |
| C57BL/6 + | 405.7 ± 8.3 | 358.8 ± 7.6 | 1.1 ± 0.02 | 746 ± 16.7 | 715 ± 16.9 | 1.0 ± 0.01 |
| vehicle (sterile | | | | | | |
| saline solution) | | | | | | |
| Rb1cKO + Vehicle | 590.8 ± 20** | 426.9 ± 18.7 | 1.4 ± 0.02* | 798 ± 22 | 582 ± 14 | 1.37 ± 0.02**** |
| (sterile saline | | | | | | |
| solution) | | | | | | |
| Rb1cKO + velusetrag | 501.9 ± 19 | 386 ± 11 | 1.2 ± 0.03 | 726 ± 32 | 662 ± 32 | 1.13 ± 0.03^{∘} |
| mg/10 mL/kg ip | | | | | | |
| Rb1cKO + velusetrag | 314 ± 12^{∘∘∘} | 328.8 ± 10 | 0.9 ± 0.02*^{∘∘∘} | 542 ± 10**^{∘∘} | 642 ± 7.8 | 0.85 ± 0.01***^{∘∘∘∘} |
| mg/10 mL/kg ip | | | | | | |

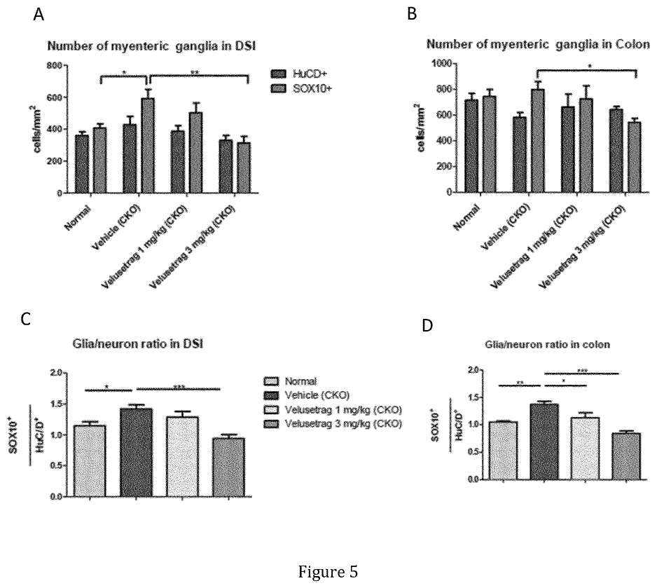

FIG. 5 shows the effect of velusetrag treatment on myenteric ganglia in DSI and proximal colon of Rb1 cKO mice (*P<0.05, **P<0.01, ***P<0.001; One way ANOVA; vs vehicle CKO).

Neuronal degeneration was also investigated by counting the number of neurons and glia cells with pan-neuronal (HuCD) or glia (SOX10) markers in intestinal whole-mount preparations of the myenteric plexus in transgenic treated and untreated mice (FIG. 14). Immunofluorescence staining showed a significant increase of glia in the proximal colon with a concomitant reduction of neuronal cells in both the small and large intestine of transgenic mice treated with vehicle compared to NC mice (P<0.001 and P<0.01, respectively). In accordance with this, the ratio of glia/Hu+ neurons was raised in both regions of the gut (P<0.001). Velusetrag treatment at both doses appeared to counteract the neuronal degeneration as shown by HuCD: glia ratio in both the small and large intestine.

### Example 7—Effect of Velusetrag on Serotonin Receptors 5-HT4 in Enteric Neurons in RB1 cKO Mice

The effect of velusetrag administration at the two tested dosages was analyzed by evaluation of the change in 5-HT4 receptor mRNA level in enteric neurons in the distal small intestine and proximal colon of wild-type and RB1 knockout mice. Total RNA was extracted from distal small intestine and proximal colon samples of mice sacrificed by CO₂ inhalation the next day of the last treatment using TRIzol Reagent (Invitrogen, US). 1 ml of TRIzol™ Reagent was added to the sample (approximately 50 mg tissue) and homogenize using Tissue Lyser (60 Hz, 1 min, precold) and incubated for 5 minutes to permit complete dissociation of the nucleoproteins complex. The obtained lysate was centrifuged for 5 minutes at 12,000×g at 4° C., then the clear supernatant was transferred to a new tube. Chloroform (0.2 mL) were added, gently mixed and incubate for 5 minutes at RT. After centrifugation for 15 minutes at 12,000×g at 4° C., the mixture was separated into a lower red phenol-chloroform, interphase, and a colorless upper aqueous phase containing the RNA which was transferred to a new tube.

By using PrimeScript RT Reagent Kit with gDNA Eraser (Takara, Japan) genomic DNA contamination was eliminated and mRNA was reverse transcribed into cDNA to be used for real-time RT-PCR (qPCR) according to the manufacturer's instructions.

The obtained cDNA was used as template in a quantitative PCR reaction (qPCR assay, Applied Biosystems, US) using primers specific for the 5-HT₄ receptor coding region into real-time PCR instrument (Bio-Rad, 1855484, US) according to the following reaction set up

| Reagent | Amount/ul |
|---|---|
| Water, nuclease-free | 3.5 |
| 2X SYBR ™ Green PCR | 5 |
| Master Mix | |
| 10 μM forward primer | 0.25 |
| 10 μM reverse primer | 0.25 |
| Template cDNA (50 ng/μl) | 1 |
| Total | 10 |

The results show that in DSI and colon, 5-HT4 receptor mRNA level slightly increase in Rb1 cKO group. After treatment with velusetrag 3 mg/kg, 5-HT4 receptor mRNA level in DSI decreased significantly. In the proximal colon no variation between the different groups is observed (Table 11).

TABLE 11
Velusetrag effect on 5-HT₄ receptor subtype

| | mRNA 5HT4 | |
|---|---|---|
| Group | DSI | Proximal colon |
| C57BL/6 + vehicle (sterile saline | 1.0 ± 0.04 | 1.0 ± 0.03 |
| solution) | | |
| Rb1cKO + Vehicle (sterile saline solution) | 1.51 ± 0.1* | 1.08 ± 0.04 |
| Rb1cKO + velusetrag 1 mg/10 mL/kg ip | 1.02 ± 0.09^{∘} | 1.10 ± 0.05 |
| Rb1cKO + velusetrag 3 mg/10 mL/kg ip | 0.87 ± 0.03^{∘} | 0.88 ± 0.02 |

Treatment with velusetrag, administered for 14 days, reduces the expression of the relevant 5-HT4 receptor in particular in the DSI region of Rb1 cKO animals.

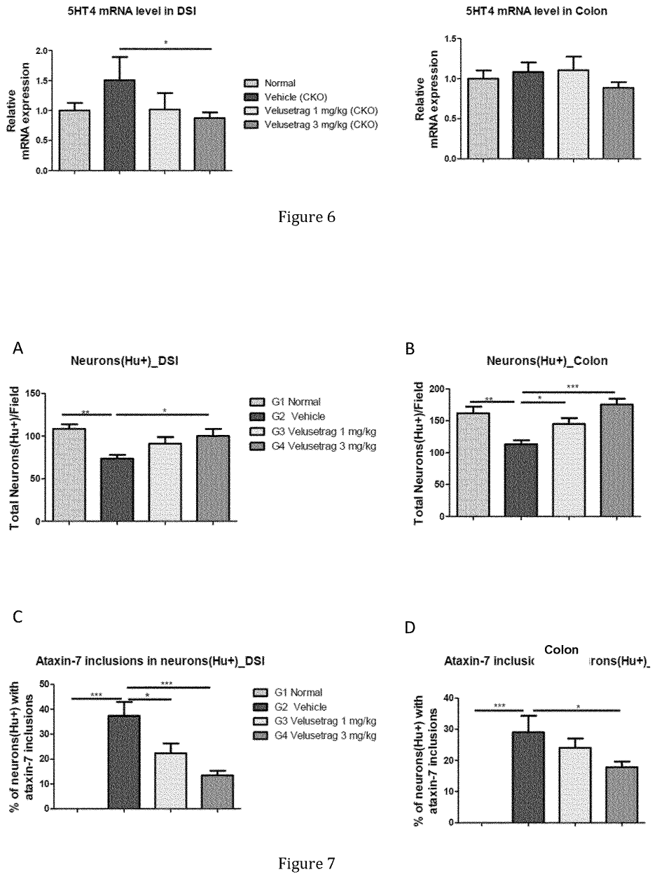

The results of the effect of velusetrag on serotonin receptor mRNA in enteric neurons of Rb1 cKO mice are represented in FIG. 6 (*P<0.05; T-test; vs vehicle CKO).

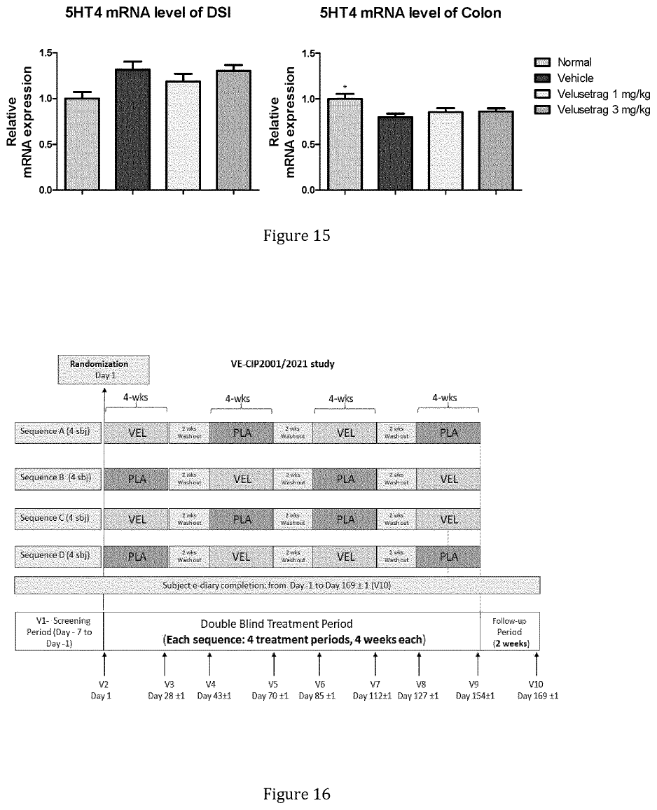

To investigate whether desensitization of the 5HT 4 receptor was apparent after 5 weeks of Velusetrag treatment, the mRNA expression levels of the 5HT4 serotoninergic receptor in DSI and colon tissues of treated and untreated transgenic groups was also carried out. Noticeably vehicle-treated transgenic mice showed significantly different 5HT4R levels compared with the control group. Such differences were not altered by Velusetrag administration within the transgenic mice (FIG. 15).

### Example 8—Effect of Velusetrag on Total Neurons and Ataxin 7 Inclusions in the Prp-SCA7-92Q Mouse Model

The presence of neurons in the myenteric plexus and the model-typical ataxin inclusions were detected by immunofluorescent analysis on DSI and proximal colon, by using primary antibodies directed to the HUC/D and Ataxin-7 proteins. Samples were prepared from animal sacrificed by CO₂ inhalation the next day of the last treatment with velusetrag as described in example 5.

After incubation in the blocking buffer, the tissue samples were incubated with HuC/D primary antibody (Abcam, US) (1:500) for one hour at room temperature followed by incubation with secondary antibody F(ab′)2-Goat anti-Rabbit HRP (Abcam, US) (1:1000) for one hour at room temperature. The tissue samples were washed with PBST (PBS+0.5% Triton X-100) for 3 times and incubated with TSA520 (1:200) for one hour at room temperature. The tissue was washed with PBST (PBS+0.5% Triton X-100) 3 times. The detection of the ataxin inclusions was performed by incubation with the primary antibody Ataxin-7 (Thermo Fisher, US) (1:2000) overnight at 4° C. and with secondary antibody F(ab′)2-Goat anti-Rabbit HRP (1:1000) for one hour at room temperature. The tissue was incubated with TSA570 ((Wi See Biotechnology, China) (1:200) for one hour at room temperature and washed with PBST (PBS+0.5% Triton X-100) for 3 times. The tissue was incubated with DAPI (1:5000) for 10 minutes at room temperature. Samples were covered with a ProLong™ Glass Antifade Mountant (Invitrogen, US) drop and images were captured with microscope (Olympus, BX53). Tissue samples were excited with excitation lamp and barrier filters (UW, BWA and GW) with excitation/emission spectra settings (340-390 nm/420IF; 460-495 nm/510-550 nm; 530-550 nm/575IF). HUC/D and Ataxin-7 antibodies combined with fluorescent dyes TSA520 and TSA570 with specific excitation and emission spectra were 488/519 nm and 555/570 nm. HuCD (green color) and co-expressed with ataxin-7 cells (yellow color) were counted.

The following primary antibodies were used: Hu C/D 1:500 (Abcam, Ab 184267, US); Ataxin-7 1:2000 (Thermo Fisher, PA1-749, US), nNOS 1:500 (Abcam, ab76067, US), ChAT 1:1000 (Abcam, Ab181023, US), Calretinin 1:500 (Merk, MAB1568, US) and SOX10 1:250 (Invitrogen, MA5-32398, US).

Secondary antibodies included F(ab′)2-Goat anti-Rabbit HRP 1:1000 (Abcam, Ab6013, US), Donkey anti-Mouse IgG (H+L) Highly Cross-Adsorbed Secondary Antibody, Alexa Fluor 488 1:500 (Invitrogen, A-21202, US), donkey anti-Rabbit IgG (H+L) Highly Cross-Adsorbed Secondary Antibody, Alexa Fluor 594 1:500 (Invitrogen, A-21207, US). TSA520 1:200 (Wi See Biotechnology, D11013, China), TSA570 1:200 (Wi See Biotechnology, D11011, China) were used for the fluorescence staining.

Data show that velusetrag is able to counteract neurodegeneration at both tested doses and in both DSI and proximal colon. Velusetrag also decrease inclusion of mutated ataxin-7 in pan neurons (Table 12).

TABLE 12
Velusetrag effect on total neurons and on ataxin-7 inclusion

| | | % neurons | | % neurons |
|---|---|---|---|---|
| | Total | Hu+ with | Total | Hu+ with |
| | neurons | ataxin-7 | neurons | ataxin-7 |
| | Hu+ | inclusions | Hu+ | inclusions |
| Group | DSI | | Proximal colon | |
| C57BL/6 + vehicle (sterile | 108.4 ± 5.4 | 0 | 158.9 ± 12 | 0 |
| saline solution) | | | | |
| Prp-SCA7-92Q + Vehicle | 73.5 ± 4.6*** | 37.2 ± 0.06**** | 113.3 ± 5.9** | 29 ± 0.05**** |
| Prp-SCA7-92Q + velusetrag | 91 ± 7.8^{∘} | 22.3 ± 0.04****^{∘} | 145.1 ± 8.9^{∘∘} | 24.1 ± 0.03**** |
| 1 mg/kg po | | | | |
| Prp-SCA7-92Q + velusetrag | 99.9 ± 2.6^{∘} | 13.6 ± 0.02****^{∘∘∘} | 175.2 ± 2.9^{∘∘∘∘} | 17.8 ± 0.02****^{∘} |
| 3 mg/kg po | | | | |

To investigate if this Tg line mice accumulate with aging neuronal inclusions positive for Ataxin-7, immunofluorescence was performed on whole mount preparations of myenteric plexus from DSI and proximal colon from 13-week-old PrP-SCA7-92Q male mice (FIGS. 7A-D). Double staining with ATX-7 and Hu C/D, a neuronal marker, revealed that about 40% of enteric neurons in DSI and 30% in the proximal colon co-localized with ataxin-7 positive nuclear inclusions. Surprisingly these percentages were significantly reduced by Velusetrag.

### Example 9—Effect of Velusetrag on Nitrergic Neurons in the Prp-SCA7-92Q Mouse Model

Enteric neurons express the enzyme nitric oxide synthase nNOS which catalyzes the production of NO from oxygen and arginine. NO acts as a neurotransmitter and is responsible for modulating gastrointestinal (GI) motility. Overproduction of NO in some inflammatory conditions also impairs normal gastrointestinal motor activity. NOS1+ neurons form close contacts with smooth muscle cells (SMCs) and interstitial cells of Cajal (ICCs); it has been observed that loss or damage of these types of neurons contributes to the development of impaired gastrointestinal motility. The presence of nitrergic neurons and ataxin inclusions was detected in the myenteric plexus of Prp-SCA7-92Q transgenic animals treated with velusetrag through immunohistochemistry analysis of DSI and proximal colon samples, using specific nNOS and Ataxin-7 primary antibodies.

Samples were prepared from C57BL/6 mice (control) and Prp-SCA7-92Q transgenic mice (Cyagen, China) with free access to water and food. Velusetrag was administered once daily at two doses (3 mg/10 mL/kg or 1 mg/10 mL/kg) by the oral route for 5 weeks starting after 8 weeks of birth until 13 weeks of age. Animal were sacrificed by CO₂ inhalation the next day of the last treatment with velusetrag as described in example 5 and processed with specific antibodies.

After fixation with 4% paraformaldehyde for 30 min, the muscle layers containing the myenteric plexus were separated from the mucosa and submucosa using fine-point forceps. Samples were washed with PBST (PBS+0.5% Triton X-100) for 3 times and incubated with blocking buffer (1.5% BSA & 1% Triton X-100 in PBS) for two hours at room temperature before incubation with nNOS primary antibody (se Abcam, US) (1:500) for one hour at room temperature. The tissue samples were washed with TBST (PBS+0.5% Triton X-100+0.5% Tween-20) 3 times and incubated with F(ab′)2-Goat anti-Rabbit HRP secondary antibody (Abcam, US) (1:1000) for one hour at room temperature. Samples were washed with PBST (PBS+0.5% Tritonx-100) 3 times and incubated with TSA520 (WI See Biotechnology, China) (1:200) for one hour at room temperature. Detection of ataxin inclusions was performed by incubation with primary antibody Ataxin-7 (Thermo Fisher, US) (1:2000) overnight at 4° C. Samples were washed with TBST (PBS+0.5% Triton X-100+0.5% Tween-20) for 3 times. Samples were incubated with F(ab′)2-Goat anti-Rabbit HRP secondary antibody (Abcam, US) (1:1000) for one hour at room temperature and washed with PBST (PBS+0.5% Triton X-100) for 3 times. After incubation with TSA570 (Wi See Biotechnology, China) (1:200) for one hour at room temperature and washed with PBST (PBS+0.5% Triton X-100) for 3 times tissues were incubated with DAPI (1:5000) for 10 minutes at room temperature. Tissues were covered with a ProLong™ Glass Antifade Mountant (Invitrogen, US) drop and images were captured with microscope (Olympus, BX53). Tissue samples were excited with excitation lamp and barrier filters (UW, BWA and GW) with excitation/emission spectra settings (340-390 nm/420IF; 460-495 nm/510-550 nm; 530-550 nm/575IF). The nNOS and ataxin-7 antibodies combined with fluorescent dyes TSA520 and TSA570 with specific excitation and emission spectra were 488/519 nm and 555/570 nm. nNOS (green color) and co-expressed with ataxin-7 cells (yellow color) were counted. Counts are shown in Table 13.

The following primary antibodies were used: nNOS 1:500 (Abcam, ab76067, US), ChAT 1:1000 (Abcam, Ab181023, US), Calretinin 1:500 (Merk, MAB1568, US) and SOX10 1:250 (Invitrogen, MA5-32398, US).

Secondary antibodies included F(ab′)2-Goat anti-Rabbit HRP 1:1000 (Abcam, Ab6013, US), Donkey anti-Mouse IgG (H+L) Highly Cross-Adsorbed Secondary Antibody, Alexa Fluor 488 1:500 (Invitrogen, A-21202, US), donkey anti-Rabbit IgG (H+L) Highly Cross-Adsorbed Secondary Antibody, Alexa Fluor 594 1:500 (Invitrogen, A-21207, US). TSA520 1:200 (Wi See Biotechnology, D11013, China), TSA570 1:200 (Wi See Biotechnology, D11011, China) were used for the fluorescence staining.

TABLE 13
Velusetrag effect on nitrergic neurons

| | Neurons | Neurons |
|---|---|---|
| | NOS+/field | NOS+ field |
| | (0.26 mm²) | (0.26 mm²) |
| Group | DSI | Proximal colon |
| C57BL/6 + vehicle | 27.1 ± 0.5 | 55.2 ± 1.9 |
| (sterile saline solution) | | |
| Prp-SCA7-92Q + Vehicle | 10.4 ± 0.3**** | 12.8 ± 0.2**** |
| Prp-SCA7-92Q + velusetrag | 22.0 ± 0.6*^{∘∘∘∘} | 31 ± 1.5**^{∘∘} |
| 1 mg/kg po | | |
| Prp-SCA7-92Q + velusetrag | 19.9 ± 0.5**^{∘∘∘} | 32 ± 1.0**^{∘∘∘∘} |
| 3 mg/kg po | | |

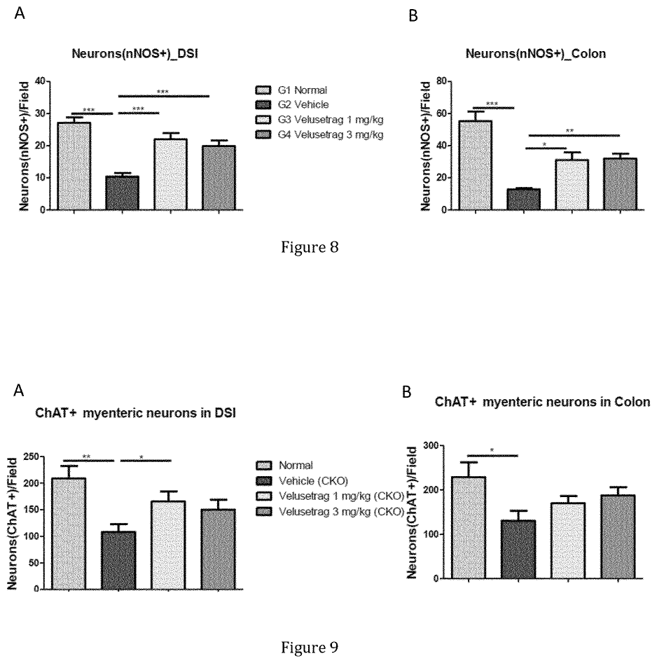

FIG. 8 shows that in the transgenic model there is a significant decrease in the number of NOS+ neurons in both regions. Further, both doses of velusetrag increase the number of nitrergic neurons when compared to the untreated transgenic mice, both regions. The effect is slightly higher in proximal colon and the effect does not appear to be dose dependent. *P<0.05, ***P<0.001; One way ANOVA; vs G2. Then, Velusetrag treatment re-established normal level of nitregic neurons.

### Example 10—Effect of Velusetrag on Cholinergic Neurons in Mouse Models

Choline acetyltransferase (CHAT) is the enzyme responsible for the biosynthesis of the neurotransmitter acetylcholine. Most acetylcholine is synthesized locally at nerve terminations where CHAT catalyzes by a single step process the transfer of an acetyl group from acetyl coenzyme A to choline. CHAT is expressed by cholinergic neurons in the central nervous system (CNS) and peripheral nervous system (PNS) and is involved in learning, memory, movement, and vision.

Immunostaining with specific CHAT antibodies is often used in morphological studies of cholinergic cell populations. Mice were treated and samples prepared as described in Example 6.

After incubation in blocking buffer the tissue samples were incubated with CHAT primary antibody (see above, Abcam, US) (1:1000) for one hour at room temperature. Samples were washed with TBST (PBS+0.5% Triton X-100+0.5% Tween-20) for 3 times and incubated with secondary antibody F(ab′)2-Goat anti-Rabbit HRP (Abcam, US) (1:1000) for one hour at room temperature. The tissue was washed with PBST (PBS+0.5% Triton X-100) for 3 times and incubated with TSA520 (Wi See Biotechnology, China) (1:200) for one hour at room temperature. The detection of ataxin inclusions was performed as described by primary antibody Ataxin-7 (Thermo Fisher, US) (1:2000) overnight at 4° C. Samples were washed with TBST (PBS+0.5% Triton X-100+0.5% Tween-20) for 3 times and then incubated with F(ab′)2-Goat anti-Rabbit HRP secondary antibody (Abcam, US) (1:1000) for one hour at room temperature and washed with PBST (PBS+0.5% Triton X-100) for 3 times. After incubation with TSA570 (Wi See Biotechnology, China) (1:200) for one hour at room temperature samples were washed with PBST (PBS+0.5% Triton X-100) for 3 times and incubated with DAPI (1:5000) for 10 minutes at room temperature. Tissues were covered with a ProLong™ Glass Antifade Mountant (Invitrogen, US) drop. Images were captured with microscope (Olympus, BX53). Tissue samples were excited with excitation lamp and barrier filters (UW, BWA and GW) with excitation/emission spectra settings (340-390 nm/420IF; 460-495 nm/510-550 nm; 530-550 nm/575IF). CHAT and Ataxin-7 antibodies combined with fluorescent dyes TSA520 and TSA570 with specific excitation and emission spectra were 488/519 nm and 555/570 nm. CHAT (green color) and co-expressed with ataxin-7 cells (yellow color) were counted. Counts detected in Rb1 cKO mice are shown in Table 14.

TABLE 14
Effect of velusetrag on CHAT neurons in Rb1 cKO mice.

ChAT+ myenteric neurons/Field
(0.41 mm²)

| | | Proximal |
|---|---|---|
| Group | DSI | colon |
| C57BL/6 + vehicle | 208.7 ± 7.5 | 228.8 ± 10.5 |
| (sterile saline solution) | | |
| Rb1cKO + vehicle | 108 ± 5.3* | 130.3 ± 7.9* |
| (sterile saline solution) | | |
| Rb1cKO + velusetrag | 165.5 ± 5.7^{∘} | 169.7 ± 4.8 |
| 1 mg/10 mL/kg ip | | |
| Rb1cKO + velusetrag | 150.1 ± 5.9 | 187.4 ± 5.9 |
| 3 mg/10 mL/kg ip | | |

Results show that the number of CHAT+ neurons in DSI and colon is significantly decreased in KO animals compared with the normal group, (DSI P<0.01; colon P<0.05). The number of CHAT neurons in DSI and in proximal colon increases following 1 mg/kg and 3 mg/kg velusetrag treatment. In particular the difference is significant in DSI following a 1 mg/kg velusetrag treatment. These results are also shown in FIG. 9 (*P<0.05, **P<0.01; T-Test; vs G2).

In Prp-SCA7-92Q transgenic mice the presence of cholinergic neurons in the myenteric plexus was assessed by immunofluorescent analysis, using the primary antibodies CHAT together with Ataxin-7, on DSI and proximal colon samples isolated from the transgenic animals.

Results are reported on Table 15.

TABLE 15
Velusetrag effect on cholinergic neurons and on ataxin-7
inclusions in Prp-SCA7-92Q transgenic mice

| | | % neurons | | % neurons |
|---|---|---|---|---|
| | Neurons | ChAT+ with | Neurons | (ChAT+) with |
| | ChAT+/field | ataxin-7 | ChAT+ field | ataxin-7 |
| | (0.26 mm²) | inclusions | (0.26 mm²) | inclusions |
| Group | DSI | | Proximal colon | |
| C57BL/6 + vehicle (sterile | 75.6 ± 2.1 | 0 | 84.4 ± 2.6 | 0 |
| saline solution) | | | | |
| Prp-SCA7-92Q + Vehicle | 53.2 ± 1.3* | 17.7 ± 0.5**** | 74.2 ± 2.3 | 33.6 ± 1.9**** |
| Prp-SCA7-92Q + velusetrag | 73.3 ± 1.8^{∘∘} | 14.3 ± 1.0**** | 94.4 ± 2.4^{∘} | 27.8 ± 1.1**** |
| 1 mg/kg po | | | | |
| Prp-SCA7-92Q + velusetrag | 67.2 ± 1.5^{∘} | 13.6 ± 0.9**** | 81.9 ± 2.4 | 24.6 ± 1.5**** |
| 3 mg/kg po | | | | |

Results show that the number of ChAT+ neurons in the Prp-SCA7-92Q model of CIPO is significantly reduced when compared to normal, wild-type mice. Further, both doses of velusetrag induce an increase in cholinergic neurons compared to the vehicle-treated transgenic mouse, both in the distal small intestine and in the proximal colon. The effect does not appear to be dose dependent.

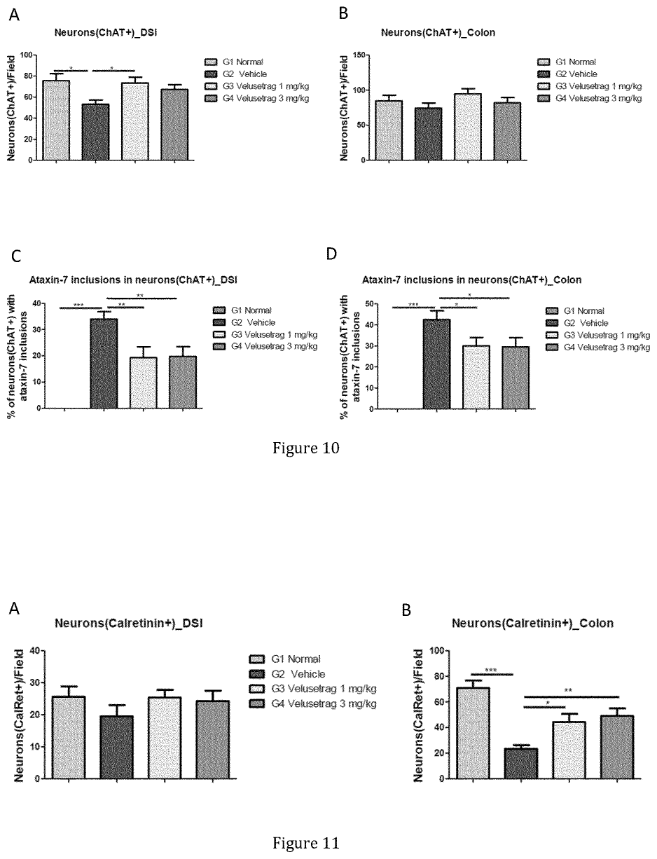

In addition, the percentage of cholinergic neurons with Ataxin 7 inclusions is significantly increased in the transgenic animals. Velusetrag at both tested doses significantly reduce the percentage of cholinergic neurons with Ataxin 7 inclusions compared to the vehicle-treated transgenic mouse. The results are shown in FIG. 10 (*P<0.05, ***P<0.001; One way ANOVA; vs G2).

Hence, velusetrag counteracts neurodegeneration of cholinergic neurons at both doses tested in both DSI and proximal colon and to reduce nuclear inclusions of mutated ataxin-7 in DSI and colon.

### Example 11—Effect of Velusetrag on Calretinin Neurons in the Prp-SCA7-92Q Mouse Model

Calretinin is a calcium-binding protein abundantly expressed in neurons. Calretinin has an important role as a modulator of neuronal excitability.

Calretinin neurons were detected and their amount measured in the myenteric plexus through immunofluorescent analysis on DSI and proximal colon samples isolated from transgenic animals. Mice were administered with velusetrag as above described and sacrificed by CO₂ inhalation. Wholemount tissue samples were prepared as described in Example 5 and washed with PBST (PBS+0.5% Triton X-100) for 3 times.

After incubation in 10% donkey serum blocking (10% donkey serum & 1% Triton X-100 in PBS) for 2 h at room temperature, tissue samples were incubated with primary antibody for Calretinin (see above Merk, US) diluted 1:500 overnight at 4° C. After washing with TBST (PBS+0.5% Tritonx-100+0.5% Tween-20) for 3 times, samples were incubated with Donkey anti-Mouse IgG (H+L) Highly Cross-Adsorbed Secondary Antibody, Alexa Fluor 488 1:500 (Invitrogen, US) for 1 h at room temperature.

After washing with PBST (PBS+0.5% Tritonx-100) for 3 times tissue samples were incubated with DAPI 1:5000 (Invitrogen, US) for 10 minutes at room temperature. Coverslip were mounted with a drop of ProLong™ Glass Antifade Mountant Tissue specimens were excited by lamp with excitation and barrier filters (UW, BWA and GW) set excitation/emission spectra (340-390 nm/420IF; 460-495 nm/510-550 nm; 530-550 nm/575IF). Calretinin antibody combined with Alexa Fluor 488 dye which specific excitation/emission spectra is 499/520 nm and 590/618 nm. Images were obtained from each animal at 20× magnification for cell counting by microscope (Olympus, BX53, Japan) using CellSens Standard Imaging software (Ver.3.2, Olympus, Japan).

Counts are reported in Table 16.

TABLE 16
Velusetrag effect on calretinin neurons
of transgenic Prp-SCA7-92Q mice

Neurons Calretinin+/
field (0.26 mm²)

| | | Proximal |
|---|---|---|
| Group | DSI | Colon |
| C57BL/6 + vehicle | 25.6 ± 1.0 | 70.9 ± 1.8 |
| (sterile saline solution) | | |
| Prp-SCA7-92Q + Vehicle | 19.5 ± 1.1 | 23.4 ± 0.9**** |
| Prp-SCA7-92Q + velusetrag | 25.4 ± 0.7 | 44.3 ± 2.0**^{∘∘} |
| 1 mg/kg po | | |
| Prp-SCA7-92Q + velusetrag | 24.3 ± 1.0 | 49.1 ± 1.8*^{∘∘∘} |
| 3 mg/kg po | | |

The number of calretinin neurons in the colon of vehicle-treated transgenic mice was significantly decreased when compared with the normal group i.e. vehicle-treated C57BL/6 mice (P<0.001). Velusetrag treatment induces an increase in the number of calretinin neurons in DSI, but no significant difference has been detected. In the colon, velusetrag 1 mg/kg and 3 mg/kg induce a significantly increase in the number of calretinin neuron (P<0.05 and P<0.01).

Variation in calretinin neurons in DSI and proximal colon of velusetrag treated PrP-SCA7-92Q transgenic mice (1 mg/kg and 3 mg/kg) is also shown in FIG. 11 (**P<0.01, ***P<0.001; One way ANOVA; vs G2 vehicle).

Calretinin expressing neurons (excitatory motor cells) were found lowered in the proximal colon but not in DSI in vehicle-treated transgenic mice whereas administration of Velusetrag protected this neuronal cell population at both the doses (FIG. 11).

Such results show that velusetrag counteracts the neurodegeneration of calretinin neurons at both doses in the proximal colon.

### Example 12—Effect of Velusetrag on n-NOS and CHAT Protein Levels in Neuromuscular Tissue in the Prp-SCA7-92Q Mouse Model

The protein level of n-NOS and CHAT in cytoplasm of proximal colon of transgenic mice was detected and analyzed by Western Blot technique.

Proximal colon samples were placed in 10 times volume (w:v) of RIPA lysis buffer (Beyotime Biotechnology, China) containing protease and phosphatase inhibitor cocktails (Thermo Scientific, US) according to the manufacturer's instructions. Homogenized the tissue on ice for 15 sec, incubated on ice for 30 min, and then centrifuged at 15,000×g for 15 min. The supernatant was collected and subjected to protein concentration analysis by BCA method. An appropriate amount of protein loading buffer was added into the supernatant for protein denaturation (95° C., 10 min). Proteins were fractionated on 10% SDS-PAGE gels and transferred to NC membranes. After blocking with 5% BSA in TBST (0.05% Tween-20) for 2 h at RT, the membranes were incubated overnight (no less than 15 h) with the primary antibody nNOS 1:1000 (Abcam, US), CHAT 1:1000 (Millipore, US), Actin 1:1000 (Beyotime, China) or MAPK 1:1000 (Cell Signaling Technology, 4695, US) at 4° C. The blots were washed with TBST (3 times, 10 min each) and incubated with secondary antibodies Goat anti-Rabbit IgG H&L (IRDye® 800CW) preadsorbed 1:5000 (Abcam, US) or Donkey anti-Goat IgG H&L (IRDye® 800CW) 1:5000 (Abcam, US) for 1 h at RT.

After wash, the membranes were placed in substrate working solution for 5 min prior to imaging using ChemiDoc System (Bio-Rad, 12003154, US). Densitometric analysis of protein bands were performed with QuantityOne software version 4.6.2. Data are shown in Table 17.

TABLE 17
Velusetrag effect on cytoplasmatic nNOS and ChAT protein levels

| | |
|---|---|
| nNOS/Actin | CHAT/Actin |
| cytoplasm | cytoplasm |

| | |
|---|---|
| Group | Proximal colon |

| | | |
|---|---|---|
| C57BL/6 + vehicle | 1.0 ± | 1.0 ± |
| (sterile saline solution) | 0.2 | 0.1 |
| Prp-SCA7-92Q + Vehicle | 0.47 ± | 0.54 ± |
| | 0.07* | 0.1* |
| Prp-SCA7-92Q + velusetrag | 1.12 ± | 0.94 ± |
| 1 mg/kg po | 0.11^{∘∘∘∘} | 0.1^{∘} |
| Prp-SCA7-92Q + velusetrag | 0.91 ± | 0.93 ± |
| 3 mg/kg po | 0.1^{∘} | 0.1^{∘} |

The levels of nNOS and ChAT proteins are significantly decreased in the Prp-SCA7-92Q model. Velusetrag reverses this effect at both doses, since an increased expression of nNOS and ChAT proteins when compared to the untreated transgenic mouse was observed in the proximal colon. The effect does not appear to be dose dependent.

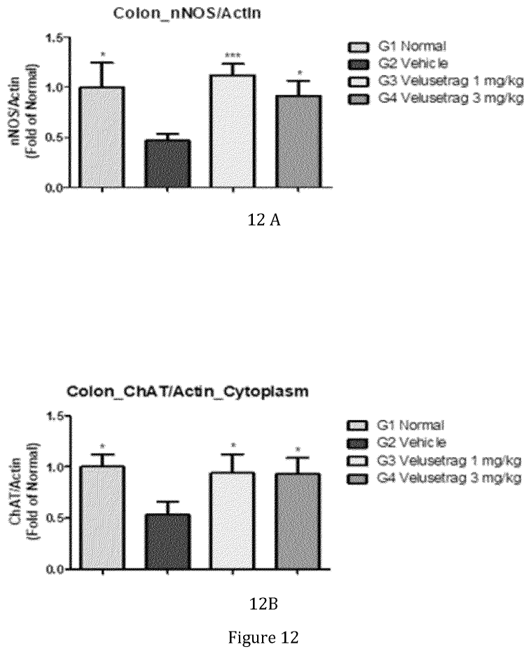

These results are graphically represented in FIG. 12 *P<0.05, ***P<0.001; T-test; vs G2

### Overall Results Obtained in Mouse Models

The efficacy of the 5-HT4 receptor agonist Velusetrag was assessed in two CIPO mouse models: RB1 cKO mice and PrP-SCA7-92Q transgenic mice. Histopathological analysis of tissues through a HE staining (HE) revealed a DSI and colon mucosa inflammation, macrophages infiltration and ulcers in both models, whereas velusetrag was able to improve the intestinal damage in a dose-dependent manner. Immunohistochemical staining revealed a significant reduction in enteric neurons of the transgenic mice as shown through HuCD antibody use. This marker of pan neurons involved in intestinal neurogenesis, neuronal survival and plasticity was decreased in distal small intestine and proximal colon of transgenic mice, thus suggesting a loss of function of Hu proteins that impact on neuronal signaling [Li et al Scientific Reports|6:38216|DOI: 10.1038/srep38216 and 25]. Moreover, pan-neurons antibody showed a significant raise of ataxin-7 inclusions in DSI and colon that was counteracted by Velusetrag treatment. This was referred, particularly to the only subpopulation of ganglion cells with ataxin-7 intranuclear inclusions expressing ChAT, but not nNOS.

Although inhibitory motor neurons that express nNOS were not affected by the ataxin-7 inclusions, they resulted lost in DSI and colon of transgenic mice respect to the control mice but protected by velusetrag administration. Neuronal NOS (nNOS) constitutively expressed in peripheral neurons are involved in synaptic plasticity, central regulation of blood pressure, smooth muscle relaxation, and vasodilatation via peripheral nitrergic nerves. The ability to form nitrergic neurons is a critical step in the development of ‘normal’ enteric circuitry and many enteric disorders would likely benefit from the transplantation and engraftment of nNOSt cells [McCann, C. J., et al., Nat Commun, 2017. 8: p. 15937.]. Nitric oxide (NO) as major non-adrenergic noncholinergic inhibitory signal in the peripheral nervous system, including the gastrointestinal (GI) tract and the enteric nervous system (ENS) relaxes GI smooth muscle to regulate physiologic peristalsis, so that the loss of neuronal NO synthase (nNOS) can disrupt normal GI motility. NO enhances transit of the rat colon by mediating descending relaxation, which in turn facilitates propulsion of the colonic contents. In the present invention, the nNOS+ neurons decreased in the ENS of the transgenic mice in vehicle-treated group, but not after velusetrag treatment, suggesting that the prokinetic drug may be beneficial for NO functions in the ENS. In the large intestine of mice, the major population of neurons expressing calretinin, calcium binding protein as marker of myenteric motor neurons, interneurons and the majority of putative primary afferent neurons muscularis mucosae and lamina propria was investigated. Also in this case, calretinin-immunoreactive neurons in myenteric ganglia (putative intrinsic primary afferent neurons) were lowered in colon of PrP-SCA7-92Q mice, whereas velusetrag was able to protect them.

Mammalian microtubule-associated protein 2 (MAP2) expressed mainly in neurons, but also in oligodendrocytes, is present at both early and late stages of neuronal development. Three isoforms, MAP2A, B, and C are known. MAP2C is localized in cell soma, dendrites, and axons of juvenile neurons, whereas MAP2A and B mainly in dendrites of mature neurons. MAP2/Tau family proteins were originally discovered for and characterized by their ability to bind and stabilize microtubules. In the present invention, intensities of MAP2 staining decreased in the KO and transgenic mice indicating there were lower numbers of mature neurons or dendrites in the transgenic mice. MAP2 intensity in ENS increased after treatment of velusetrag, suggesting that velusetrag may improve microtubule function of dendrites. Enteric glial cells were taken into account because they interact with other gastrointestinal cell types such as those of the epithelium and immune system to preserve homeostasis (Boesmans et al Frontiers in cell and Development Biology Volume 9|Article 775102).

In vitro studies suggested that Sox10-expressing undifferentiated progenitors in embryonic gut produce both enteric neurons (Sox10−) and glia (Sox10+), but the neurogenic potential of these cells in vivo and its temporal regulation during gut organogenesis are currently unclear.

A large proportion of submucosal HuCD+ neurons, as well as a small subpopulation of myenteric neurons, were found to co-express the glial markers Sox10 and S100B (Parathan P. et al. (2020). The Enteric Nervous System Undergoes Significant Chemical and Synaptic Maturation during Adolescence in Mice. Dev. Biol. 458, 75-87. 10.1016/j.ydbio.2019.10.011). It is likely that these are newly differentiated neurons which are in the process of switching off glial marker expression.

In the present invention an increase in number of glia in proximal colon respect to the control mice was observed. Taking into account the ration of glia out of pan neurons the raise in KO or transgenic mice either in DSI and colon, was inhibited by Velusetrag treatment at both the doses. Kulkarni et al. (Proc Natl Acad Sci USA, 2017. 114 (18):: p. E3709-E3718) showed that, under physiological conditions, the adult ENS is maintained by a dynamic balance between neuronal apoptosis and neurogenesis from neuronal Sox10−/Nestin+ precursors that are not mature glial cells. However, the neurogenic potential of Sox10/+Nestin− cells is only activated upon injury. In the present invention with the KO and transgenic mice an increase of Sox10+ glial cells was observed. However, Velusetrag treatment resulted in a decrease of Sox10+ glial cells. It is plausible that in the transgenic mice an increase in glial cells may be needed to compensate for the reduction of some other types of neuron cells. As Velusetrag can boost the production of the neuron cells, less glial cells are needed when the intestine injury or dysfunction attenuated.

Because Velusetrag is a 5-HT4 receptor (5-HT4R) agonist, 5-HT4R expression was investigated, as this type of receptor 5-HT4 can regulate gastrointestinal movement and reduce visceral sensitivity. Furthermore, 5-HT4R is implicated in the development and functional maintenance of enteric nerves. In the present invention, variations of 5HT4R mRNA level were observed in the cKO and transgenic mice, with increase in intestine tissues and decrease in colon tissues. Apparently, Velusetrag treatment did not alter the mRNA levels of 5HT4 in the transgenic mice.

In the present invention, the biomarkers related to cellular survival, proliferation, and metabolism (the signaling mTOR/AKT) were also evaluated. The mammalian target of rapamycin (mTOR), serine/threonine protein kinase belonging to the phosphatidylinositol 3-kinase (PI3K)-related kinase (PIKK) family interacts with other subunits to form two distinct complexes, mTORC1 and mTORC2. mTORC1 coordinates the cell growth and metabolism in response to environmental input, including growth factors, amino acids, energy and stress. mTORC2 mainly controls cell survival and migration through phosphorylating glucocorticoid-regulated kinase (SGK), protein kinase B (Akt), and protein kinase C (PKC) kinase families. The dysregulation of mTOR was found in many human diseases including cancer, cardiovascular diseases, neurodegenerative diseases, and epilepsy. In the present invention an activation of signaling mTOR as phosphorylation was observed in DSI of transgenic and cKO mice and this was counteracted by Velusetrag administration. AKTs serine-threonine kinases with three different protein isoforms (AKT1, AKT2, and AKT3) also act on cellular survival, proliferation, and metabolism. Also in this case a phosphorylation of AKT was found in DSI of transgenic and cKO mice, whereas it was reduced by Velusetrag treatment. At last, p70S6 kinase is activated by growth factors and plays a central role in cell growth and proliferation by mediating the phosphorylation of the 40S ribosomal protein, S6, thereby enabling efficient translation of 5-terminal oligopyrimidine tract mRNAs (5-TOPs). mTOR promotes translation initiation by its phosphorylation of two targets, ribosomal p70S6 kinase (S6K1) and eukaryotic translation initiation factor 4E binding protein 1 (4E-BP1). In the present invention, again an increased p70S6 activation in transgenic and cKO mice was observed. This effect was inhibited by Velusetrag. On the whole, Velusetrag at 3 mg/kg group, was able to decrease p-mTOR, p-Akt, p-P70S6 expressions in distal small intestine after 5 weeks treatment.

Velusetrag significantly improve the biomarkers and ameliorate the pathological conditions of CIPO, therefore holding high therapeutic potentials for treatment of CIPO.

### Example 13—Multicenter, Double-Blind, Placebo-Controlled, Two Treatment Four Period Cross-Over, Multiple (n=1) Trial to Evaluate the Efficacy and Safety of Velusetrag in Patients with CIPO

### Material and Methods

A phase II, multicenter, double-blind, placebo-controlled, two treatment four period cross-over, multiple (n=1) trial was made to evaluate the efficacy and safety of velusetrag 15 mg once a day, compared to placebo, in subjects with idiopathic Chronic Intestinal Pseudo-Obstruction (CIPO) or CIPO secondary to primary neurodegenerative or demyelinating conditions.

This study evaluated the safety, tolerability and efficacy of velusetrag 15 mg once a day versus placebo in improving symptom severity associated with CIPO in subjects with idiopathic CIPO and CIPO secondary to neurodegenerative conditions.

CIPO is a chronic, rare disease, with fluctuating symptoms and differences in underlying pathology, which may respond differently to active treatment. Therefore, a standard parallel group placebo-controlled study would not have allowed the detection of a clear benefit due to the large variability of clinical presentation.

An n=1 cross over, placebo-controlled study design allowed the assessment of treatment effect in every subject, thereby allowing individual subjects to act as their own control and evidencing a positive effect even in a single subject, avoiding under-estimation of therapeutic efficacy (Emmanuel et al. Randomised clinical trial: the efficacy of prucalopride in patients with chronic intestinal pseudo-obstruction—a double-blind, placebo-controlled, cross-over, multiple n=1 study. Aliment Pharmacol Ther. 2012 January; 35 (1): 48-55).

A placebo was used as comparator because it allowed the estimation of the real treatment effect of velusetrag.

The study consisted of a screening period of up to 7 days (Day −7 to Day −1) followed by 4 periods of treatment of 4 weeks each wherein subjects were treated with either velusetrag (VEL) 15 mg (2 periods) or placebo (PLA) (2 periods) with a wash-out period of 2 weeks between treatment periods and 2 weeks of follow-up (total of approximatively 175 days), as reported in FIG. 16. Visits where assessed at the beginning of the screening period (Visit 1), at the beginning and at the end of each period of treatment (Visit 2 to Visit 9) and at the end of the follow-up period (Visit 10).

After the up to 7-days screening period (from Day −7 to Day −1), at the randomization visit (Visit 2), eligible subjects were randomly allocated in a 1:1:1:1 ratio to one of the following four sequences, as reported in FIG. 16:
A. VEL-PLA-VEL-PLA
B. PLA-VEL-PLA-VEL
C. VEL-PLA-PLA-VEL
D. PLA-VEL-VEL-PLA
   wherein VEL=velusetrag 15 mg once daily for 4 weeks.
   PLA=matching placebo once daily for 4 weeks.

Gastrointestinal symptom severity (abdominal pain, bloating, nausea and vomiting) and bowel habit (weekly recall) were registered on Day −1 and weekly after randomization, during both treatment and washout periods, as appropriate according to the Schedule of Assessments using an e-diary until end of follow up period.

During the study the following parameters were collected during the visits:
serum nutritional markers (albumin, pre-albumin, vitamin B12 and folate) levels were collected at pre-treatment and at the of each 2-week washout period and at the end of follow-up after the fourth period or at early termination visit (ETV) or at early switch visit (ESV);
the number of pseudo-obstruction episodes and hospitalizations were assessed at each visit;
the need for parenteral and enteral nutrition supplements were assessed at each visit;
a lactulose breath test (L-BT) was performed to assess orocecal transit time at the screening visit (Visit 1) and at the end of the first 4-week treatment period (Visit 3);

Table 18 reports the Schedule of Assessments of the measured parameters.

TABLE 18
Schedule of Assessments

| | | 1^{st} Treatment | | | | | | | | |
|---|---|---|---|---|---|---|---|---|---|---|
| | | Period | | 2^{nd} Treatment | | 3^{rd} Treatment | | 4^{th} Treatment | | EFU |
| | Screening | Randomi | | Period | | Period | | Period | | End of |
| | Period | z. SOT-1 | EOT-1 | SOT-2 | EOT-2 | SOT-3 | EOT-3 | SOT-4 | EOT-4 | follow-up |
| | Visit | | | | | | | | | |
| | V1 | V2 | V3 | V4 | V5 | V6 | V7 | V8 | V9 | V10 |
| | Day | | | | | | | | | |
| | −7 to −1 | 1 | 28(±1) | 43(±1) | 70(±1) | 85 (±1) | 112(±1) | 127(±1) | 154(±1) | 169(±1) |
| Vital Signs | X | X | X | X | X | X | X | X | X | X |
| Waist | | X | X | X | X | X | X | X | X | X |
| circumference | | | | | | | | | | |
| recording | | | | | | | | | | |
| Body Weight | X | | X | X | X | X | X | X | X | X |
| Physical | X | | X | X | X | X | X | X | X | X |
| Examination | | | | | | | | | | |
| Triplicate 12lead | X | X | X | X | X | X | X | X | X | X |
| ECG | | | | | | | | | | |
| Clinical | X | | X | X | X | X | X | X | X\| | X |
| Chemistry, | | | | | | | | | | |
| Urinalysis and | | | | | | | | | | |
| Hematology | | | | | | | | | | |
| Prealbumin, | X | | | X | | X | | X | | X |
| albumin, Vitamin | | | | | | | | | | |
| B12 and folate | | | | | | | | | | |
| Lactulose Breath | X | | X | | | | | | | |
| Test | | | | | | | | | | |
| Concomitant | X | X | X | X | X | X | X | X | X | X |
| medications | | | | | | | | | | |
| Pseudo- | X | | X | X | X | X | X | X | X | X |
| obstruction | | | | | | | | | | |
| episodes | | | | | | | | | | |
| recording | | | | | | | | | | |
| Hospitalizations | X | | X | X | X | X | X | X | X | X |
| recording | | | | | | | | | | |
| Artificial food | X | | X | X | X | X | X | X | X | X |
| need scale | | | | | | | | | | |
| Adverse Events | X | X | X | X | X | X | X | X | X | X |
| SF-12 | | X | X | X | X | X | X | X | X | |
| Collection of | | X | X | | | | | | | |
| samples for proof | | | | | | | | | | |
| of absorption | | | | | | | | | | |

SOT: Start of Treatment; EOT: End of Treatment; EFU: End of Follow up; SF-12: Short-Form 12 items health survey.

The subject also had to register the permitted medications for CIPO gastrointestinal symptoms taken right before the start of the first treatment period (Day −1) as well as all changes in treatment doses (increased/reduced) or number of concomitant drugs during the entire treatment and washout periods. In addition, investigational product intake and time were registered by the subjects in the e-diary during the treatment periods daily.

### Study Population

A total of 17 patients with history of chronic idiopathic intestinal pseudo-obstruction or CIPO secondary to neurodegenerative or demyelinating disease were randomized to be allocated to one of the four arms described before.

Randomization occurred at Visit 2 (V2-Day 1), after all screening procedures had been performed and eligibility for the study had been assessed.

Randomization was stratified by CIPO diagnosis (idiopathic or secondary to neurodegenerative or demyelinating disease) and by 5-HT₄ receptor agonist responder status (responder/naive or non-responder) as follows:
5-HT4 receptor agonist non-responder.
5-HT4 receptor agonist responder/naïve and idiopathic CIPO
5-HT4 receptor agonist responder/naïve and CIPO secondary to neurodegenerative or demyelinating disease.

Non-responders were defined as all subjects who, based on investigator's judgement, had a history of a lack of benefit from 5-HT4 receptor agonists

### Main Inclusion Criteria:

1. Men or women aged 18-80 years.
2. Subjects with history of chronic idiopathic intestinal pseudo-obstruction or CIPO secondary to neurodegenerative or demyelinating disease.
3. Subjects with estimated oral caloric intake of at least 30% of the daily age- and sex-recommended caloric intake (stage 0, 1 or 2 of the “artificial food need” scale, according to Table 19).

TABLE 19
Artificial food need scale

| | Type of diet | |
|---|---|---|
| | followed by | |
| Stage | the subject | Description: |
| 0 | Free Oral | Adequate caloric and hydro-electrolytic |
| | | intake with the frequency of meals and food |
| | | consistency unchanged compared to the |
| | | habits prior to the onset of the disease. |
| | | Unnecessary oral supplements. |
| 1 | Modified oral | Oral caloric and hydro-electrolytic intake |
| | in the absence | still adequate thanks to: changed meal |
| | of non-oral | frequency with respect to habits prior to |
| | integration | the onset of illness AND/OR use of food of |
| | | reduced consistency AND/OR use of oral |
| | | supplements. |
| 2 | Oral with the | Oral caloric and hydro-electrolytic intake |
| | need for non- | possible but inadequate with the need for |
| | oral integration | parenteral integration or via feeding tube |
| 3 | Full non-oral | Oral caloric and hydro-electrolytic intake |
| | | not possible with the need for parenteral |
| | | nutrition and/or via feeding tube. |

4. Subjects with at least 2 out of 4 CIPO gastrointestinal symptoms (i.e., abdominal pain, bloating nausea and vomiting), each of the 2 with a score ≥3 (on a 0 to 4 scale) collected on the gastrointestinal symptom questionnaire at Day −1.
5. Subjects accepting to provide and legally capable of providing free and informed consent to all procedures included in the protocol.
6. All sexually active male participants who are partner of women of childbearing potential had to use a condom during intercourse until the 90th day after the end of the entire study.
7. All female participants had to be:
   a. of non-childbearing potential, i.e.: i) post-menopausal (at least 2 years without spontaneous menses), or ii) surgically sterile (bilateral tubal occlusion, or hysterectomy), or iii) ablation of both ovaries
OR
   b. of childbearing potential with a negative pregnancy test result at screening and randomization AND agreeing to use a highly effective method of contraception (i.e., with failure rate of less than 1% per year) until the end of the entire study.
   Highly effective methods of contraception were defined according to the recommendations of the EU Clinical Trial Facilitation Group

### Main Exclusion Criteria

1. Subjects with primary CIPO or CIPO secondary to other known endocrine/metabolic, autoimmune diseases and neurologic conditions other than neurodegenerative or demyelinating diseases.
2. Subjects with conditions characterized by mechanical intestinal obstruction.
3. Nasogastric tube, gastrostomy tube, or jejunostomy feeding tube in place at randomization or planned throughout the duration of the study, or artificial food need scale stage 3 (“total non-oral nutrition”, see Artificial Food Need” (AFN) Scale for CIPO subjects in 21.3 Appendix 3 of the study protocol).
4. Presence of untreated clinically relevant thyroid dysfunction or known thyroid dysfunction not well controlled by treatment (e.g., subjects with abnormal thyroid stimulating hormone [TSH], and, if available, triiodothyronine [T3] and thyroxin [T4] levels) deemed clinically significant by the Investigator.
5. Subjects with history of diabetes at screening.
6. Clinically significant ECG abnormalities (e.g., ST segment elevation or depression suggestive of ischemia, partial or complete left bundle branch block [LBBB]) at Screening and randomization.
7. Screening ECG with a QTcF>450 msec in males or >470 msec in females or family history of sudden cardiac death.
8. Subjects requiring a low galactose diet.
9. Hypersensitivity or documented intolerance to lactulose, lactose or any excipient of the lactulose preparation to be used for L-BT.
10. History of sensitivity to velusetrag, or any of the velusetrag or placebo excipients.
11. Use of scopolamine or erythromycin within 2 weeks prior to Screening and/or planned throughout the duration of the study.
12. Use of 5-HT4 receptor agonists (e.g., prucalopride, cisapride, clebopride and cinitapride) within 5 days prior to randomization and/or planned throughout the duration of the study.
13. Use of opioids within 8 weeks from screening and/or planned throughout the duration of the study.
14. Received strong cytochrome P450-isozyme 3A4 (CYP3A4) inhibitors (e.g., clarithromycin, indinavir, itraconazole, ketoconazole, nefazodone, nelfinavir, ritonavir, saquinavir, telithromycin, grapefruit juice) or strong CYP3A4 inducers (e.g., rifampin, phenytoin, carbamazepine, phenobarbital, St. John's wort) within 2 weeks prior to screening and/or planned throughout the duration of the study.
15. Received strong P-glycoprotein (P-gp) transporter inhibitors (e.g., captopril, carvedilol, diltiazem) within 2 weeks prior to Screening and/or planned throughout the duration of the study.
16. Received strong breast cancer resistance protein (BCRP) transporter inhibitors (e.g., curcumin, cyclosporine A, eltrombopag) within 2 weeks prior to Screening and/or planned throughout the duration of the study.
17. Current swab-positive or suspected (under investigation) COVID19 infection.
18. Cancer (excluding non-melanoma skin cancer) and/or need of any anti-cancer treatment (also including radiotherapy) within the last 5 years.
19. Severe kidney impairment (i.e., estimated glomerular filtration rate <30 ml/min).
20. Aspartate aminotransferase (AST) or alanine transaminase (ALT) levels >2.5 times the upper limit of normal (ULN); bilirubin (unless deemed to be due to Gilbert's Syndrome) or alkaline phosphatase (ALP)>1.5 times ULN.
21. Severe hepatic impairment defined as Child-Pugh C.
22. History of any of the following cardiac disorders:
   a. Torsade de pointes, ventricular tachycardia, ventricular fibrillation.
   b. Previous myocardial infarction, unstable angina pectoris, acute coronary syndrome, coronary artery or cerebral revascularization procedure or stroke within the previous 18 months.
   c. Angina pectoris class 2-4 during the last 12 months prior to screening.
   d. Congestive heart failure NYHA class III-IV during the last 18 months prior to screening.
23. History of any alcohol or drug abuse or dependence within the last year (Investigator's judgement).
24. Any current significant health condition (e.g., cardiovascular, respiratory, renal, hepatic, neurologic, psychiatric, hematologic, oncologic, immune, muscle and joint, etc.) that in the Investigator's judgement may:
   a. jeopardize the subject's safe participation in the trial; or
   b. make unlikely the subject's completion of the study; or
   c. make unlikely the subject's compliance with the study procedures (e.g., highly anticipated need of non-permitted treatments, significant disability, terminal illness).
25. Pregnant or breastfeeding woman.
26. Use of any experimental drug within 12 weeks prior to screening.

### Composition Used

Subjects enrolled in the study took 15 mg (3×5 mg capsules) of velusetrag or matching placebo (3 capsules) once daily during the 4 weeks of each of the 4 treatment periods. Subjects were instructed to take three capsules of medication orally, once daily at approximately the same time each morning, on an empty stomach, with water.

### Velusetrag

Active product ingredient: velusetrag (ScinoPharm Taiwan, Ltd.) 5 mg.
Excipients: lactose monohydrate, microcrystalline cellulose, hypromellose and magnesium stearate.

### Placebo

Active product ingredient: absent.
Excipients: lactose monohydrate, microcrystalline cellulose, hypromellose and magnesium stearate.

### Prohibited Medications

Use of the following medications was not allowed within 2 weeks prior to Screening and/or planned throughout the duration of the study:
Scopolamine
Erythromycin.
Strong CYP3A4 inhibitors or strong CYP3A4 inducers (according to Table 20)
Strong P-gp transporter inhibitors (according to Table 20)
Strong BCRP transporter inhibitors (according to Table 20)

TABLE 20
Prohibited medications

| Category | Excluded Agents (not exhaustive list) |
|---|---|
| Strong | boceprevir, cobicistat, clarithromycin, conivaptan, danoprevir, |
| CYP3A4 | diltiazem, elvitegravir, erythromycin, grapefruit juice, idelalisib, |
| Inhibitors | indinavir, itraconazole, ketoconazole, lopinavir, mibefradil, |
| | nefazodone, nelfinavir, paritaprevir and ombitasvir and/or dasabuvir, |
| | posaconazole, ritonavir, saquinavir, telithromycin, troleandomycin, |
| | telaprevir, tipranavir, voriconazole. |
| Strong | carbamazepine, enzalutamide, mitotane, phenobarbital, phenytoin, |
| CYP3A4 | rifampin, St. John's wort. |
| Inducers | |
| Strong P-gp | amiodarone, captopril, carvedilol, clarithromycin, conivaptan, |
| Inhibitors | cyclosporine A, diltiazem, dronedarone, itraconazole, lapatinib, |
| | lopinavir, propafenone, quinidine, ranolazine, ritonavir, saquinavir, |
| | telaprevir, ticagrelor, tipranavir, verapamil. |
| Strong BCRP | Curcumin, cyclosporine A, eltrombopag |
| Inhibitors | |

In addition, use of:
Opioids was not allowed within 8 weeks from screening and/or planned throughout the duration of the study.
5-HT4 receptor agonists (e.g., prucalopride, cisapride, clebopride, cinitapride) were not allowed starting from 5 days before randomization and throughout the duration of the study.
Orally poorly absorbed opioids (i.e., loperamide) could be used if medically indicated (treatment of potential AEs).

If a 5-HT4 receptor agonist (e.g., prucalopride, cisapride, clebopride, cinitapride) or scopolamine, or erythromycin and an opioid for analgesic use was taken during the study, the subject was considered a treatment failure and was withdrawn from the study.

Additional prescription and over-the-counter medications were permitted, provided that such agents are not known to be strong inducers or inhibitors of CYP3A4, P-gp and BCRP activity.

Any addition or change in regimen of concomitant medications that affect CYP3A4, P-gp and BCRP activity were in accordance with inclusion or exclusion criteria and recorded in the source documents and eCRF.

Medications taken 30 days prior to the Screening visit through the end of the Follow-up period were recorded. If subjects had previously taken a 5-HT4 receptor agonist (e.g., prucalopride, cisapride, clebopride, cinitapride), the last treatment period (even if occurred more than 30 days before the screening visit) and the efficacy of such treatment (responder/not responder) for each subject was recorded in the eCRF.

If clinically permitted, subjects were encouraged not to change their current regimen of concomitant medications or not to start new concomitant medications.

### Permitted Treatments

Medication(s) used to relieve main symptoms of CIPO were allowed and the use of the following concomitant medications were recorded in the e-diary daily:
Treatments for nausea and vomiting and/or non-serotoninergic prokinetics (e.g., metoclopramide, domperidone, pharmaceutical ginger preparations, pyridostigmine, prochlorperazine, promethazine, ondansetron and aprepitant).
Treatments for constipation (e.g., macrogol, bisacodyl linaclotide, laxative enemas).
Treatments for diarrhea (e.g., tannate, loperamide).
Treatments for abdominal pain (e.g., paracetamol. NSAIDs, trimebutine, mebeverine, gabapentin, duloxetine, amitriptyline.).
Others (e.g., octreotide, somatostatin, pancreatic enzymes, probiotics, rifaximin, metronidazole, fluconazole).
Permitted medications for CIPO gastrointestinal symptoms taken right before the start of the first treatment period (Day −1) as well as all changes in concomitant treatment doses or number of concomitant drugs during the entire treatment and washout periods, until end of follow up, were recorded daily in the e-diary.

### Efficacy Endpoints

### Primary Efficacy Endpoints

The Primary efficacy endpoints was to evaluate the change in weekly global gastrointestinal symptoms average index score (WGGSAIS) from start to the end of each treatment period. The score was obtained by averaging the scores for each of the 4 symptoms assessed weekly: abdominal pain, bloating, nausea and vomiting on a scale from 0 to 4 (0—Absent; 1—Mild (not influencing usual activities); 2—Moderate (diverting from, but not urging modification of, usual activities); 3—Severe (influencing usual activities markedly enough to urge modifications; 4—Extremely severe (precluding daily activities)) (Barbara G et al, 2004, Gastroenterology March; 126(3):693-702).

### Secondary Efficacy Endpoints

The secondary efficacy endpoints were evaluated as the following changes from the start to the end of each treatment period:
1. Change in waist circumference.
2. Proportion of subjects with 1-point improvement in WGGSAIS.
3. Change in individual symptoms score for abdominal pain, bloating, nausea, vomiting.
4. Change in number of weekly bowel movements (only in subjects with Bristol stool scale type 1 or 2 at the start of the treatment period).
5. Change in number of weekly complete evacuations (only in subjects with Bristol stool scale type 1 or 2 at the start of the treatment period).
6. Change in stool type at the Bristol stool scale.
7. Change in weekly bowel habit satisfaction score measured using a scale from 0 to 10.
8. Change from start in orocecal transit time measured using lactulose breath test (only at the end of the first treatment period).
9. Change in nutritional markers (serum albumin, pre-albumin, vitamin B12 and folic acid levels) from start of each treatment period to the end of each 2 week wash out period (or follow up period).
10. Proportion of days with change in permitted medications used to relieve main CIPO gastrointestinal symptoms during each treatment and wash out period (or follow up period):
   a. Proportion of days with dose increased compared to the start of the period.
   b. Proportion of days with dose decreased compared to the start of the period.
   c. Proportion of days with drug added compared to the start of the period.
   d. Proportion of days with drug removed compared to the start of the period.
11. Change in quality of life (short-form 12 items health survey-SF-12).
12. Number of CIPO-related hospitalizations during the treatment periods.
13. Change in stage of the “artificial food need” scale.
14. Number of pseudo-obstruction episodes based on investigator's judgement.
15. Change from the end of each treatment period to the ends of the first and the second week of wash out (or follow up period) in:
a. weekly abdominal pain score.
b. weekly bloating score.
c. weekly nausea score.
d. weekly vomiting score.
e. weekly global gastrointestinal symptoms average index score.
f. number of weekly bowel movements (only in subjects with Bristol stool scale type 1 or 2 at the start of the treatment period).
g. number of weekly complete evacuations (only in subjects with Bristol stool scale type 1 or 2 at the start of the treatment period).
h. stool type at the Bristol stool scale.
i. weekly bowel habit satisfaction score measured using a scale from 0 to 10.

### Safety Assessments

Safety was assessed at each visit measuring and evaluating changes from the baseline of the following parameters:
Vital signs (including blood pressure, pulse, temperature).
Physical examination also including height and weight.
Routine laboratory parameters (hematocrit, hemoglobin, red blood cell count, white blood cell count with differential count and platelet count for hematology; glycaemia, total cholesterol, triglycerides, serum creatinine, urea or blood urea nitrogen [BUN], sodium, potassium, chloride, AST, ALT, Gamma-GT, alkaline phosphatase, total and fractioned bilirubin (direct and indirect), erythrocyte sedimentation rate, PT (or INR), aPTT for biochemistry; specific gravity, pH, protein, glucose, ketones, hemoglobin, nitrite, bilirubin, urobilinogen and microscopic examination for urinalysis).
Triplicate ECG
Adverse events (AEs).
Withdrawal from study due to AEs.

### Statistical Method

### Determination of Sample Size

The sample size was based on the main analysis (t-test) of the primary endpoint, i.e., the differences of WGGSAIS among an evaluable pair. A pair is considered evaluable when there is an evaluation available for a subject in a cycle, i.e. a consecutive velusetrag and placebo period treatment, or viceversa.

Of note, each subject should be evaluated twice, once for each evaluable pair. Consequently, each subject can contribute to 0, 1 or 2 pairs and only data that constitute a pair evaluable for primary endpoint were considered in this analysis.

### Definition of Main Study Populations

Safety Set (SS): all treated subjects. Analysis on the SS was performed according to the actual treatment received.
Modified-Full Analysis Set 1 (mFAS1): all subjects responder/naïve to 5HT4 receptor agonist randomized and treated and with data on the primary endpoint at least once during a velusetrag treatment period and at least once during a placebo treatment period in the same cycle.
Modified-Full Analysis Set 2 (mFAS2): all subjects randomized and treated and with data on the primary endpoint at least once during a velusetrag treatment period and at least once during a placebo treatment period in the same cycle.
Per Protocol Set (PPS): all subjects in the mFAS1 who fulfilled the study protocol requirements in terms of compliance to treatment and collection of primary efficacy data and with no major deviations.

### Primary Efficacy Endpoint Analysis

The WGGSAIS was obtained by averaging the scores for each of the 4 symptoms assessed weekly: abdominal pain, bloating, nausea and vomiting, with lower scores representing better health. If at least 2 symptoms were assessed, the average score was calculated, otherwise, it was considered missing.

Considering the evaluable pairs (i.e., evaluation available both for velusetrag and placebo within cycle), the WGGSAIS is summarized by treatment considering data that were collected from pre-treatment to the end of each period (4 weeks), along with changes that occurred during the wash-out period, i.e. indicating the WGGSAIS of the post-treatment period and of following pre-treatment period.

Difference of WGGSAIS between velusetrag and placebo treatment were computed within each evaluable pair as changes in velusetrag minus changes in scoring occurred during treatment with placebo (for both, velusetrag and placebo, the changes are between the end of treatment and pre-treatment values). A paired t-test evaluated scoring differences between velusetrag and placebo treatment: results were provided in terms of mean differences with relative 95% CI and p-value.

A supportive analysis was performed through a mixed model in which the outcome was the difference between velusetrag and placebo treatment WGGSAIS in changes from pre-treatment to end of treatment in the same cycle considering cycle as fixed effect, and subject as random effect. Results of Type III tests of fixed effect and the estimate of difference between velusetrag and placebo of changes from pre-treatment to end of treatment are provided together with 95% CI.

These analyses were provided on the mFAS1 without any missing data imputation as primary analysis.

Sensitivity analysis: the same analyses were provided also on the mFAS2.

### Main Secondary Efficacy Endpoints Analysis

Proportion of subjects with 1-point improvement in WGGSAIS

Change between the end of treatment and pre-treatment WGGSAIS value≤−1 point was computed and considered a “success” while changes >−1 were considered a “failure”. The proportion of subjects with a 1-point improvement in WGGSAIS is summarized by treatment sequence, period and timepoint (weekly evaluation by e Diary.

Considering the evaluable pairs, the proportions of pairs of observations with 1-point improvement in WGGSAIS is summarized by treatment and timepoint. Odds Ratio of achieving the 1-point improvement is provided for velusetrag vs. placebo treatment together with the correspondent 95% CI using logistic model for end of treatment timepoint. Fisher test was applied to compare pairs of observations “success”/“failure” between treatments.
Change in individual symptoms score

Each symptom (abdominal pain, bloating, nausea, vomiting) was analyzed as described for the primary efficacy endpoint analysis.
Effect on bowel habit in subject with constipation (i.e., Bristol Stool Type 1 and 2 at Pre-Treatment)

The number of bowel movements and complete evacuations were reported weekly in the eDiary together with the evaluation of stool consistency according to Bristol scale They are summarized by treatment considering data that were collected from pre-treatment to the end of each treatment period together with changes.
Stool consistency

Average stool consistency was recorded according to Bristol scale on an eDiary. The Bristol scale was categorized as follows: constipation: Type 1 and 2; normal: Type 3 and 4; diarrhea: Type 5, 6 and 7. The distribution of subjects according to category from pre-treatment to end of treatment is provided. Considering the evaluable pairs, the number of subjects according to category at pre-treatment of each cycle is provided together with the number of subjects who remain or change category at the end of a treatment period of each cycle. In addition, the Fisher Exact test to compare the distribution according to category at the end of treatment was performed and the p-value provided.
Orocecal transit time

Orocecal transit time was computed based on the lactulose breath test (L-BT) performed only during the first treatment period. Changes in orocecal transit time were computed between post- and pre-treatment. A t-test to evaluate differences between velusetrag and placebo was performed.
Pseudo-obstruction episodes

Considering the evaluable pairs, the number of pseudo-obstruction episodes occurring within each treatment period and number of pseudo-obstruction episodes occurring within wash-out/follow up was considered as categorical variables classified as 0, 1, 2 and >2 and are summarized by treatment. In addition, the Fisher Exact test to compare the distribution of the number of pseudo-obstruction episodes occurring within each treatment period was performed considering the categories and the p-value provided.
Effect of discontinuing treatment with velusetrag at the end of each treatment period, during each 2-week wash out period (or follow up period)

### Definition

The effect of discontinuing treatment was evaluated considering the following set of variables previously described:
Weekly abdominal pain score
Weekly bloating score
Weekly nausea score
Weekly vomiting score
Weekly global gastrointestinal symptoms average index score
Number of weekly bowel movements (only in subjects with Bristol stool scale type 1 or 2 at pre-treatment)
Number of weekly complete evacuations (only in subjects with Bristol stool scale type 1 or 2 at the start of the treatment period)
Stool type on the Bristol stool scale
Weekly bowel habit satisfaction score measured using a scale from 0 to 10

The same descriptive analyses described above are repeated focusing on change from the end of each treatment period to the end of each week of washout period or follow-up period instead of the change between post-treatment and pre-treatment.

### Safety Statistical Analysis

### Adverse Events (AE)

Adverse events starting on or after the first intake of investigational product were considered treatment-emergent AEs (TEAEs). Verbatim terms for AEs were coded using the most current version of Medical Dictionary for Regulatory Activities (MedDRA) coding. In addition, events were classified as velusetrag emergent if the last treatment taken before the start of the AE was velusetrag, or placebo emergent if the last treatment taken before the start of the AE was placebo.

Absolute and relative frequency of subjects with treatment-related TEAEs, Serious TEAEs, treatment-related Serious TEAEs, TEAEs leading to treatment temporary interruption, TEAEs leading to treatment discontinuation, Serious TEAEs leading to treatment temporary interruption, Serious TEAEs leading to treatment discontinuation and Fatal TEAEs were provided.

### Laboratory Parameters

Laboratory values (hematology, biochemistry and urinalysis), including change from baseline for continuous parameters are summarized descriptively for each scheduled visit. Frequency tables were produced for categorical parameters. Clinical laboratory values outside normal ranges are listed. For selected laboratory data, marked abnormalities are summarized using shift tables.

### Electrocardiogram

Triplicate interpretable ECG recordings were performed and the average of the three readings used to determine ECG parameters (e.g., HR, PR, QRS, QT, QTcF). ECGs were reviewed at the clinical center and final interpretation of all ECGs completed by a central reviewer and sent to the site for evaluation and filing. A listing of subjects with 12-lead ECG results considered as “abnormal and not clinically significant” or “abnormal and clinically significant” was provided.

### Results

### Study Population

17 subjects were randomized. 16 subjects completed the study (mFAS2 population) and 1 subject discontinued the study due to Informed consent form withdrawal after the first month of treatment.

Of the 16 randomized subjects who completed the study, one randomized subject did not previously respond to 5-HT4 receptor agonists while the other 15 were 5-HT4 receptor agonist responders or naïve (mFAS1 population). With regard to 5-HT₄ responder status, 10 subjects (66.67%) had a history of benefit from 5-HT₄ receptor agonists and 5 subjects (33.33%) had never been treated (naïve).

All subjects were diagnosed with idiopathic CIPO. In the 6 months prior to consent, the mean number of pseudo-obstruction episodes and of CIPO-related hospitalizations were 1.8±2.34 (range: 0; 6) and 0.3±0.62 (range: 0; 2), respectively.

Compliance in the mFAS1 population was always greater than 80% in all treatment sequences and periods. Similar results were observed in the mFAS2 and PPS.

Demographic features for the mFAS1 population (primary population) are shown in

### Table 21.

On average, age was 56.7±10.65 years (range: 40; 74 years), most subjects were female (12, 80.00%) and not Hispanic or of Latino ethnicity (11, 73.33%).

TABLE 21
Demography; modified Full Analysis Set 1

| | VEL-PLA- | PLA-VEL- | VEL-PLA- | PLA-VEL- | |
|---|---|---|---|---|---|
| | VEL-PLA | PLA-VEL | PLA-VEL | VEL-PLA | TOTAL |
| Age (years) | | | | | |
| N | 4 | 4 | 4 | 3 | 15 |
| Mean (SD) | 51.5 (6.61) | 59.3 (13.35) | 57.5 (9.68) | 59.3 (15.57) | 56.7 (10.65) |
| Median | 52.0 | 63.5 | 56.5 | 61.0 | 59.0 |
| Q1; Q3 | 47.0; 56.0 | 50.5; 68.0 | 50.5; 64.5 | 43.0; 74.0 | 47.0; 66.0 |
| Range | 43; 59 | 40; 70 | 47; 70 | 43; 74 | 40; 74 |
| Gender, n (%) | | | | | |
| Male | 0 | 2 (50.00) | 1 (25.00) | 0 | 3 (20.00) |
| Female | 4 (100.00) | 2 (50.00) | 3 (75.00) | 3 (100.00) | 12 (80.00) |
| Ethnicity, n (%) | | | | | |
| Hispanic or Latino | 1 (25.00) | 1 (25.00) | 2 (50.00) | 0 | 4 (26.67) |
| Not Hispanic or Latino | 3 (75.00) | 3 (75.00) | 2 (50.00) | 3 (100.00) | 11 (73.33) |

PLA = Placebo; Q1 = first quartile; Q3 = third quartile; SD = Standard deviation, VEL = Velusetrag.
Percentages are computed on modified Full Analysis Set 1.

### Efficacy Endpoints

### Primary Efficacy Endpoints

A summary of the primary efficacy results is shown in the table below.

Mean WGGSAIS was consistently lower at end of treatment than at pre-treatment for both velusetrag and placebo, indicating an improvement in symptoms. A greater decrease was constantly observed for velusetrag.

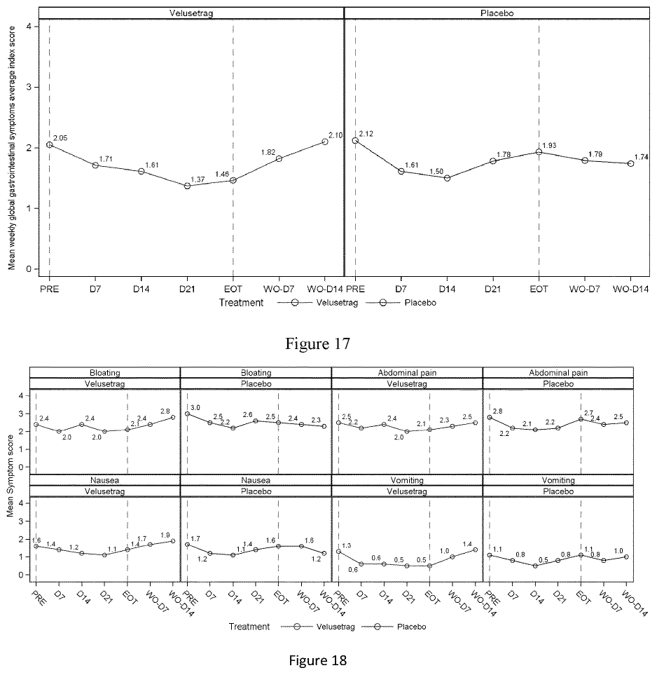

FIG. 17 shows the line plot of the mean of WGGSAIS by treatment (30 observed pairs); mFAS1 population.

Table 22 reports the Summary of WGGSAIS by treatment in the mFAS1 population (30 observed pairs).

TABLE 22
Summary of WGGSAIS by treatment (30 observed pairs); mFAS1 population

| | | | | | | Change from pre-treatment-treatment | | | | | |
|---|---|---|---|---|---|---|---|---|---|---|---|
| | | | | | | | Base | | | | |
| | n | Mean | SD | Min | Max | N | Mean | Mean | SD | Min | Max |
| Velusetrag | | | | | | | | | | | |
| Pre- | 30 | 2.047 | 0.8371 | 0.50 | 3.75 | — | — | — | — | — | — |
| treatment- | | | | | | | | | | | |
| treatment | | | | | | | | | | | |
| Day 7 | 29 | 1.707 | 0.7291 | 0.50 | 3.75 | 29 | 2.066 | −0.359 | 0.7434 | −2.25 | 1.00 |
| Day 14 | 30 | 1.608 | 0.6113 | 0.50 | 2.75 | 30 | 2.047 | −0.439 | 0.7264 | −2.17 | 1.50 |
| Day 21 | 30 | 1.367 | 0.7303 | 0.00 | 2.75 | 30 | 2.047 | −0.681 | 0.7694 | −2.67 | 1.00 |
| End of | 30 | 1.458 | 0.6857 | 0.00 | 3.00 | 30 | 2.047 | −0.589 | 0.7790 | −2.67 | 0.75 |
| treatment | | | | | | | | | | | |
| Placebo | | | | | | | | | | | |
| Pre- | 30 | 2.124 | 0.6012 | 0.75 | 3.50 | — | — | — | — | — | — |
| treatment- | | | | | | | | | | | |
| treatment | | | | | | | | | | | |
| Day 7 | 29 | 1.612 | 0.8388 | 0.00 | 3.75 | 29 | 2.107 | −0.495 | 0.5525 | −1.75 | 0.37 |
| Day 14 | 28 | 1.503 | 0.6863 | 0.00 | 3.25 | 28 | 2.084 | −0.581 | 0.6693 | −2.00 | 0.75 |
| Day 21 | 30 | 1.783 | 0.6940 | 0.00 | 3.00 | 30 | 2.124 | −0.341 | 0.7282 | −2.50 | 1.25 |
| End of | 30 | 1.925 | 0.7661 | 0.00 | 3.25 | 30 | 2.124 | −0.199 | 0.7362 | −2.25 | 1.75 |
| treatment | | | | | | | | | | | |

mFAS1 = modified Full Analysis Set 1; SD = Standard deviation.
“n” refers to the number of observed pairs for the evaluation of primary endpoint (i.e., paired differences of velusetrag minus placebo within paired treatment cycle available). Each subject can contribute with 0, 1 or 2 pairs

Borderline situations can be considered for the approaches with missing imputation also on the mFAS2 and PPS.

In mFAS2 the mean change from pre-treatment to end of treatment was −0.529±0.7956 (range: −2.67; 0.75) for velusetrag and −0.187±0.7137 (range: −2.25; 1.75) for placebo, resulting in a mean difference between treatments of −0.342+0.9343 (range: −2.92; 1.50). The decrease was thus greater for velusetrag (paired t-test: p-value=0.0469, 95% CI for the mean difference in changes between velusetrag and placebo, equal to −0.679; −0.005; mixed model: p-value=0.0563, 95% CI for the mean difference in changes between velusetrag and placebo, equal to −0.69; 0.01).

### Secondary Endpoints

Proportion of Subjects with at Least a 1-Point Improvement in WGGSAIS

Subjects with available baseline/reference missing data value greater than or equal to 1 were identified as evaluable to reach at least a 1-point improvement.

Based on the WGGAIS value from pre-treatment, at each timepoint a “success” indicates the presence of at least a 1-point improvement in WGGAIS while a “failure” indicates the absence of at least a 1-point improvement.

Among the 15 subjects in the mFAS1, a total of 20 evaluable observed pairs were identified for the evaluation of “success”/“failure” to reach a 1-point improvement in WGGAIS.

Table 23 reports the proportion of pairs with a 1-point improvement in WGGAIS by treatment at each timepoint for 20 evaluable observed pairs of the mFAS1 population.

At end of treatment, the proportion of pairs with “success” was 30.00% for velusetrag (6 pairs) and 5.00% for placebo (1 pair). A greater probability of “success” for velusetrag was observed (Fisher Exact test: p-value=0.0915; Odds Ratio estimate from the logistic model=8.14, 95% CI of 0.88; 75.48).

These data suggest that velusetrag is more effective than placebo in improving the analyzed symptoms, although a placebo effect was detectable.

TABLE 23
Proportion of pairs of observations with 1-point improvement
in WGGAIS by treatment; mFAS1 (20 evaluable observed pairs)

| Pairs of observations with | | | | |
|---|---|---|---|---|
| 1-point improvement in | | | | |
| weekly global gastrointestinal | Velusetrag | | Placebo | |
| symptoms average index score | n (%) | | n (%) | |
| Day 7 | | | | |
| Success | 5 | (25.00) | 3 | (15.00) |
| Failure | 14 | (70.00) | 17 | (85.00) |
| Missing | 1 | (5.00) | 0 | |
| Day 14 | | | | |
| Success | 3 | (15.00) | 5 | (25.00) |
| Failure | 17 | (85.00) | 15 | (75.00) |
| Day 21 | | | | |
| Success | 6 | (30.00) | 4 | (20.00) |
| Failure | 14 | (70.00) | 16 | (80.00) |
| End of treatment | | | | |
| Success | 6 | (30.00) | 1 | (5.00) |
| Failure | 14 | (70.00) | 19 | (95.00) |

| | | | |
|---|---|---|---|
| Odds Ratio (95% CI) | 8.14 | (0.88; 75.48) | reference |

| | |
|---|---|
| Fisher Exact Test: p-value | 0.0915 |

CI = Confidence Interval
Odds Ratio and Fisher Exact Test refers to End of treatment.
Only data that constitute pair evaluable for primary endpoint are considered in the analysis.

### Change in Individual Symptoms Score

Each individual symptom among bloating, abdominal pain, nausea and vomiting was monitored during the treatment.

FIG. 18 shows the mean of each individual symptom score by treatment sequence and period from pre-treatment to the end of each treatment period considering also wash-out/follow-up evaluation for a total of 23 observed pairs in the mFAS1 population.

Of the four symptoms monitored, vomiting showed a statistically significant improvement.

In subjects administered with velusetrag, the mean value of vomiting symptom score decreased from 1.3±1.58 (range: 0; 4) at pre-treatment to 0.5±0.95 (range: 0; 3) at end of treatment.

In subjects administered with placebo, the mean vomiting symptom score remained stable: 1.1±1.42 (range: 0; 4) at pre-treatment and 1.1±1.25 (range: 0; 4) at end of treatment.

The mean difference between velusetrag and placebo in changes between pre-treatment and end of treatment was −0.8±1.44 (range: −4; 1), which indicates a greater decrease for velusetrag (paired t-test: p-value=0.0164, 95% CI equal to −1.4; −0.2; mixed model: p-value=0.0177, 95% CI equal to −1.4; −0.2).

Similar results were observed when performing the same analysis on the mFAS2 population (24 observed pairs) for each individual symptom score. In particular, for vomiting a slight decrease from pre-treatment to end of treatment was observed during treatment with velusetrag, while scores during treatment with placebo remained stable, resulting in a statistically significant difference between treatments.

Effect on Bowel Habit (i.e., Weekly Bowel Movements and Weekly Complete Evacuations) in Subject with Constipation

Among the 15 subjects in the mFAS1 population, 4 subjects with constipation were identified as having Bristol stool scale type equal to 1 or 2 as at least one pre-treatment value. Six pairs were counted among these 4 subjects to analyze the effect of treatment on bowel habit considering the number of weekly bowel movements and the number of weekly complete evacuations.

In subjects treated with velusetrag, the mean number of weekly bowel movements increased from 3.2±1.72 (range: 1; 6) at pre-treatment to 4.8±2.14 (range: 2; 8) at end of treatment, with a mean change of 1.7±2.34 (range: −1; 5). In the same group of treatment, the mean number of weekly complete evacuations increased from 1.5±1.05 (range: 0; 3) at pre-treatment to 2.2±1.17 (range: 1; 4) at end of treatment, with a mean change of 0.7±1.21 (range: −1; 2).

In subjects treated with placebo, the mean number of weekly bowel movements increased from 2.3±1.03 (range: 1; 4) at pre-treatment to 3.2±1.17 (range: 3; 5) at end of treatment, with a mean change of 0.8±0.98 (range: 0; 2). In the same group of treatment, the mean number of weekly complete evacuations was 1.0±1.26 (range: 0; 3) at pre-treatment and 1.5±1.05 (range: 0; 3) at end of treatment, resulting in a mean change of 0.5±0.84 (range: 0; 2).

For both treatments, the number of weekly bowel movements and number of weekly complete evacuations thus increased, with a slightly greater increase for velusetrag.

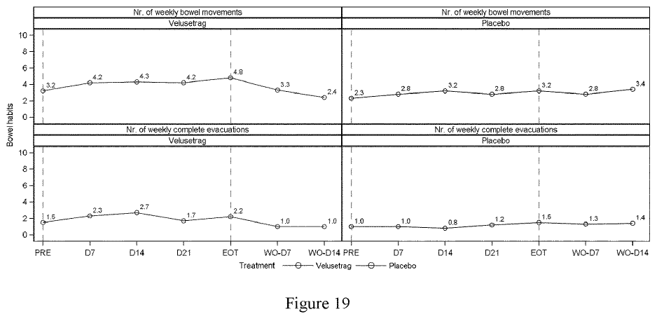

FIG. 19 shows the line plot of the mean of weekly bowel movements and weekly complete evacuations for treatment in the mFAS1 population.

### Stool Consistency

Subjects of the mFAS1 population was stratified also for stool consistency, classified according to Bristol stool scale as.
“Constipation” (i.e., Bristol scale of Type 1 or 2)
“Normal” (i.e., Bristol scale of Type 3 or 4).
“Diarrhea” (i.e., Bristol scale of Type 5, 6 or 7)

In the mFAS1 population it was observed 23 evaluable pairs.

The distribution of stool consistencies among the 23 observed pairs were similar for velusetrag and placebo at pre-treatment.

In subjects administered with velusetrag, the frequency of normal feces increased to 26.09% at the end of treatment, while the frequency of constipation decreased to 0% the end of treatment. Small fluctuations were observed in subjects administered with placebo.

In subjects administered with velusetrag, all 4 pairs with constipation and 1 of the 16 pairs with diarrhea at pre-treatment had normal stools at end of treatment.

In subjects administered with placebo, 2 of the 17 pairs with diarrhea at pre-treatment had normal stools, 2 were constipated and 13 continued to have diarrhea at end of treatment. Of the 3 pairs with normal stools at pre-treatment, 1 had diarrhea at end of treatment.

In conclusion, for velusetrag the frequency of subjects who had normalized feces increased, while the frequency of constipation decreased. Small fluctuations were observed for placebo.

Table 24 reports the shift of stool consistency between end of treatment and pre-treatment by treatment in mFAS1 population (23 evaluable observed pairs).

TABLE 24
Stool consistency: shift table between end of treatment and pre-
treatment by treatment (23 evaluable observed pairs); mFAS1

| | Velusetrag | | | | Placebo | | | |
|---|---|---|---|---|---|---|---|---|
| | Constipation | Normal | Diarrhea | Total | Constipation | Normal | Diarrhea | Total |
| | n (%) | n (%) | n (%) | n (%) | n (%) | n (%) | n (%) | n (%) |
| Bristol | 4 | 3 | 16 | 23 | 3 | 3 | 17 | 23 |
| scale class | (17.39) | (13.04) | (69.57) | (100.00) | (13.04) | (13.04) | (73.91) | (100.00) |
| at the pre- | | | | | | | | |
| treatment | | | | | | | | |
| Bristol | | | | | | | | |
| scale class | | | | | | | | |
| at the end | | | | | | | | |
| of treatment | | | | | | | | |
| Constipation | 0 | 0 | 0 | 0 | 3 | 0 | 2 | 5 |
| | | | | | (100.00) | | (11.76) | (21.74) |
| Normal | 4 | 1 | 1 | 6 | 0 | 2 | 2 | 4 |
| | (100.00) | (33.33) | (6.25) | (26.09) | | (66.67) | (11.76) | (17.39) |
| Diarrhea | 0 | 2 | 15 | 17 | 0 | 1 | 13 | 14 |
| | | (66.67) | (93.75) | (73.91) | | (33.33) | (76.47) | (60.87) |
| Total | 4 | 3 | 16 | 23 | 3 | 3 | 17 | 23 |
| | (100.00) | (100.00) | (100.00) | (100.00) | (100.00) | (100.00) | (100.00) | (100.00) |
| Fisher | | | | | | | | 0.0628 |
| Exact Test: | | | | | | | | |
| p-value | | | | | | | | |

“n” refers to the number of observed pairs for the evaluation of stool consistency. Each subject can contribute with 0, 1 or 2 pairs.

The same analysis was carried on mFAS2 population, considering 24 pairs. A similar frequency distribution of pairs classified as “Constipation”, “Normal” and “Diarrhea” was observed at the pre-treatment.

In subjects administered with velusetrag, 0, 6 (25.00%) and 18 (75.00%) pairs were classified as “Constipation”, “Normal” and “Diarrhea”, respectively.

In subjects administered with placebo, 6 (25.00%), 4 (16.67%) and 14 (58.33%) pairs were classified as “Constipation”, “Normal” and “Diarrhea”, respectively.

For this population, a statistically significant difference in stool consistency emerged between pre-treatment and end of treatment (p-value=0.0339).

### Pseudo-Obstructions

The evaluation of the number of pseudo-obstruction episodes both during treatment and wash-out period was made considering the 15 subjects in the mFAS1 (30 observed pairs).

Pseudo-obstructions occurred once in 2 pairs on velusetrag arm treatment (6.67%) and in 7 pairs on placebo arm treatment (23.33%), while it occurred twice in 1 pair (3.33%) on velusetrag arm treatment. During wash-out, only 1 pair (3.33%) experienced 1 pseudo-obstruction following treatment with placebo.

Table 25 reports the distribution of pairs according to the number of pseudo-obstruction by treatment, wherein “Pseudo-obstruction episodes during treatment period” refers to data collected in the “end of treatment” visit, while “Pseudo pseudo-obstruction episodes during wash-out period” refers to data collected in the “start of treatment” visit.

In conclusion the Pseudo-obstruction episodes were lower during velusetrag treatment compared to placebo: 4 episodes in the velusetrag group and 8 episodes in the placebo group.

TABLE 25
Summary of number of pseudo-obstruction episodes
by treatment (30 observed pairs); mFAS1

| | Velusetrag | | Placebo | |
|---|---|---|---|---|
| | n (%) | | n (%) | |
| Number of pseudo-obstruction episodes | | | | |
| during treatment period | | | | |
| 0 | 27 | (90.00) | 23 | (76.67) |
| 1 | 2 | (6.67) | 7 | (23.33) |
| 2 | 1 | (3.33) | 0 | |
| Fisher Exact Test: p-value | 0.1455 | | | |
| Number of pseudo-obstruction episodes | | | | |
| during wash-out period | | | | |
| 0 | 30 | (100.00) | 29 | (96.67) |

| | | | |
|---|---|---|---|
| 1 | 0 | 1 | (3.33) |

“n” refers to number of observed pairs for the evaluation of pseudo-obstruction episodes. Each subject can contribute with 0, 1 or 2 pairs.

### Orocecal Transit Time

Orocecal transit time was computed based on the lactulose breath test (L-BT). The L-BT test was performed only during the first treatment period of the study.

In the mFAS1 population, in subjects administered with velusetrag mean orocecal transit time was 169.3±60.44 min (range: 90; 240 min) at Visit 1 (Screening) and 162.9±67.32 min (range: 90; 240 min) at Visit 3 (end of treatment −1), resulting in a decrease of −6.4±118.70 min (range: −150; 150 min).

In the same population, in subjects administered with placebo mean orocecal transit time was 167.5±61.79 min (range: 90; 240 min) at Visit 1 and 173.6±52.58 min (range: 105; 240 min) at Visit 3, with an increase of 10.0±55.50 min (range: −60; 105 min).

The between-treatment difference of changes of −16.4 (95% C: −133.0; 100.1)

Table 26 reports a summary of orocecal transit time by treatment together with changes between post-treatment (i.e., end of first treatment period at Visit 3 (end of treatment-1)) and pre-treatment (i.e., Screening value at Visit 1) on the mFAS1 population.

The same analysis was repeated excluding subjects who had taken antibiotics in the two weeks preceding LB-T executions as sensitivity analysis. Five subjects were excluded as well as 1 subject who had an LB-T but orocecal transit time could not be computed. Also in this case, a decrease in orocecal transit time (min) between Visit 3 and Visit 1 was observed with a higher mean change (−63.0±83.79 min for velusetrag and 18.8±68.60 min for placebo, mean change between velusetrag and placebo −81.8). These data suggest a prokinetic activity of velusetrag.

TABLE 26
Summary of orocecal transit time by treatment; mFAS1

| | | | | | | Change from Visit 1 (Screening | | | | | |
|---|---|---|---|---|---|---|---|---|---|---|---|
| | | | | | | | Base | | | | |
| | n | Mean | SD | Min | Max | N | Mean | Mean | SD | Min | Max |
| Orocecal transit | | | | | | | | | | | |
| time (min) | | | | | | | | | | | |
| Velusetrag | | | | | | | | | | | |
| Visit 1 | 7 | 169.3 | 60.44 | 90 | 240 | — | — | — | — | — | — |
| (Screening) | | | | | | | | | | | |
| Visit 3 (EOT-1) | 7 | 162.9 | 67.32 | 90 | 240 | 7 | 169.3 | −6.4 | 118.70 | −150 | 150 |
| Placebo | | | | | | | | | | | |
| Visit 1 | 6 | 167.5 | 61.79 | 90 | 240 | — | — | — | — | — | — |
| (Screening) | | | | | | | | | | | |
| Visit 3 (EOT-1) | 7 | 173.6 | 52.58 | 105 | 240 | 6 | 167.5 | 10.0 | 55.05 | −60 | 105 |
| p-value | | | | | | | | | | | 0.7622 |

SD = Standard deviation, EOT = End of Treatment.
“n” refers to number of subjects

### Effect of Treatment Discontinuation

Considering eDiary data collected during wash-out or follow-up, changes from end of treatment are described on the pairs identified. Considering the WGGAIS for each symptom, slight increases in mean were observed for velusetrag, indicating a worsened condition, while for placebo the means remained stable. Similarly, for WGGSAIS total score, the mean change at the end of wash-out was 0.54±0.736 for velusetrag and −0.06±0.815 for placebo. For placebo, changes in stool consistency were observed in terms of improvement for 2 constipated pairs who then had normal stool, and in terms of worsening for the 4 normal pairs that became constipated (2) or had diarrhea (2). For velusetrag, changes were observed in terms of improvement for 1 pair with diarrhea who then had normal stool, and in terms of worsening for the 3 normal pairs that became constipated (2) or had diarrhea (1). In subjects with constipation, and for both the number of weekly bowel movements and number of weekly complete evacuations, a slight decrease from end of treatment to end of wash-out was observed for velusetrag and placebo, with a slightly greater decrease for velusetrag.

### Safety Evaluation

The Safety Set included 17 subjects; 7 (41.18%) had a total of 29 TEAEs classified as velusetrag emergent (the last treatment taken before the TEAE onset date was velusetrag), whereas 10 subjects (58.82%) had a total of 38 TEAEs classified as placebo emergent (the last treatment taken before the TEAE onset date was a placebo). No deaths, serious TEAEs, or TEAEs leading to treatment discontinuation or treatment interruption were reported.

Also, no treatment-related TEAEs were observed and no cardiovascular adverse reactions were observed.

Velusetrag was found to be safe and well-tolerated in all regimens studied.

1. A method for the treatment of a condition selected from the group consisting of a) idiopathic chronic intestinal pseudo-obstruction (CIPO), b) neuropathic chronic intestinal pseudo-obstruction, c) chronic intestinal pseudo-obstruction being secondary to neurodegeneration or being secondary to autoimmune conditions or being secondary to connective tissue disorders or being secondary to demyelinating conditions, comprising administering an effective amount of 1-isopropyl-2-oxo-1,2-dihydroquinoline-3-carboxylic acid {(1S,3R,5R)-8-[(R)-2-hydroxy-3-(methanesulfonyl-methyl-amino)propyl]-8-azabicyclo[3.2.1]oct-3-yl}amide (velusetrag) or a pharmaceutically acceptable salt thereof to a patient in need thereof.

2. The method of claim 1, wherein the pharmaceutically acceptable salt is a hydrochloride salt.

3. The method of claim 1, wherein velusetrag is in crystalline form and/or hydrated form.

4. The method of claim 1, wherein the idiopathic CIPO, the neuropathic CIPO or CIPO being secondary to neurodegeneration or being secondary to autoimmune conditions or being secondary to connective tissue disorders or being secondary to demyelinating conditions is one caused by diseases of the autonomic nervous system such as stroke, encephalitis, calcification of basal ganglia, orthostatic hypotension, one caused by diseases of intestinal wall nervous system such as paraneoplastic syndrome, viral infections, iatrogenic disorders, Hirschsprung's disease, Chagas' disease, Von Recklinghausen's disease, one caused by diseases of the intestinal wall muscle layer such as myotonic dystrophy, progressive systemic sclerosis or one caused by diseases of the mixed enteric nervous system and smooth muscle layer such as scleroderma, dermatomyositis, amyloidosis, Ehler-Danlos syndrome or one caused by an unknown mechanism such as hypothyroidism, hypoparathyroidism, pheochromocytoma, antidepressants drugs, antineoplastics or bronchodilatators or one caused by immune-mediated and connective tissue disorder or disease such as paraneoplastic disease (CNS neoplasms, lung microstoma, bronchial carcinoid, leyomyosarcomas, systemic lupus erythematosus.

5. The method of claim 1, wherein the patient is an adult or a pediatric patient.

6. The method of claim 5, wherein the patient is one having a history of chronic CIPO or of CIPO secondary to neurodegenerative disease or demyelinating disease.

7. The method of claim 1, wherein at least one of the symptoms of CIPO selected from abnormal gastrointestinal motility, increased dilatation of the proximal colon and/or of the distal small intestine, modified intestinal contractility, ulceration, inflammation of the proximal colon and/or of the distal small intestine, pseudo-obstructive episode, vomiting, bloating, abdominal pain, mental health, quality of life and lethality is alleviated and/or ameliorated and/or wherein the number and/or frequency of CIPO-related and/or CIPO-caused hospitalizations is reduced.

8. The method of claim 1, wherein the velusetrag is administered in a dosage of 0.5 to 30 mg/day based on the weight of the free base.

9. The method of claim 1, wherein the velusetrag is administered for a period of two to twelve weeks.

10. The method of claim 1, wherein the velusetrag is administered orally, parenterally, buccally, sublingually, rectally, intraperitoneally, or endotracheally.

11. The method of claim 10, wherein the percutaneous administration is subcutaneous, intramuscular, intravenous, transdermal, or by implantation.

12. The method of claim 10, wherein the velusetrag is administered orally in the form of a liquid, capsule, tablet, chewable tablet, dissolvable film, pill, lozenge, cachet, dragee, powder, granules, a solution, a suspension, an oil-in-water or water-in-oil liquid emulsion, an elixir or a syrup.

13. The method of claim 10, wherein the velusetrag is administered parentally in the form of a liquid, solid or gel.

14. The method of claim 1, wherein the velusetrag is taken orally with or without food.

15. The method of claim 8, wherein the velusetrag is administered 5 to 15 mg/day, based on the weight of the free base.

16. The method of claim 8, wherein the velusetrag is administered 15 mg/day, based on the weight of the free base.

17. The method of claim 9, wherein the velusetrag is administered for a period of two to six weeks.

18. The method of claim 14, wherein the velusetrag is taken in a once daily administration.
